(19) United States
(12) Patent Application Publication
(10) Pub. No.: US 2019/0125753 A1
(43) Pub. Date: May 2, 2019

(54) COMPOUNDS AND METHODS FOR INCREASING HEMATOPOIESIS

(71) Applicant: University of Southern California, Los Angeles, CA (US)

(72) Inventors: Yi Zhao, South Pasadena, CA (US); Michael McMillan, Alhambra, CA (US); Yusuke Higuchi, Los Angeles, CA (US); Michael Kahn, Altadena, CA (US); Cu D. Nguyen, Azusa, CA (US)

(21) Appl. No.: 16/095,290

(22) PCT Filed: Apr. 20, 2017
(86) PCT No.: PCT/US17/28508

§ 371 Date: Oct. 19, 2018

Related U.S. Application Data
(60) Provisional application No. 62/325,445, filed on Apr. 20, 2016.

(51) Int. Cl. A61K 31/53; A61K 31/519; A61P 7/06

(52) U.S. Cl. CPC A61K 31/53; A61P 7/06; A61K 31/519

(57) ABSTRACT

Provided herein is a compound of Formula (I) or a pharmaceutically acceptable salt thereof, wherein R¹ is hydrogen or C₁-C₆ alkyl; ring A is cyclohexyl or phenyl, optionally substituted; ring B is aryl or a nitrogen-containing heteroaryl, optionally substituted; ring C is phenyl substituted with hydroxyl; each L¹, L² and L³ is independently C₁-C₆ alkyl, —CONR²— or —CONR²—X—C₁-C₆ alkyl-, each optionally substituted; R² is hydrogen or C₁-C₆ alkyl; and X is a bond or a 5-6 membered heterocycle containing up to 3 ring heteroatoms; and methods of use such as a method for increasing hematopoiesis, for enhancing expansion of a hematopoietic stem cell (HSC), or for inhibiting an interaction between a β-, and/or γ-catenin protein in a cell or a subject by administering a compound of Formula (I) to the subject.

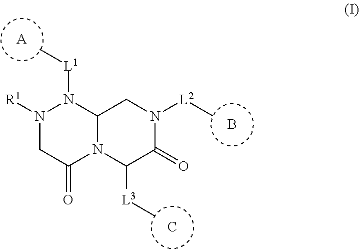

### CROSS REFERENCE WITH RELATED APPLICATION

This application claims the benefit of U.S. Provisional Patent Application No. 62/325,445, filed Apr. 20, 2016, the disclosure of which is incorporated herein in its entirety.

### FEDERAL FUNDING ACKNOWLEDGEMENT

Support from USC Norris Comprehensive Cancer Center Support Grant P30 CA014089 (MK), NIH 1R01CA166161-01A1, 1R21NS074392-01, 1R21AI105057-01 and NIH 1R01 HL112638-01 (MK) is gratefully acknowledged.

### TECHNICAL FIELD

Provided herein are compounds, compositions and methods for increasing hematopoiesis in vivo and expanding hematopoietic stem cells (HSC) in vitro and in vivo.

### BACKGROUND

A subject having reduced levels of hematopoiesis may not generate sufficient quantities of new blood cells to remain or become healthy. Compositions and methods are needed for increasing hematopoiesis in patients who suffer from, or are susceptible to, depressed hematopoiesis activity or reduced blood cell levels. Such decreased or depressed hematopoiesis activity or reduced blood cell levels may be caused, for example, by chemotherapy, radiation therapy, radiation exposure, accidental radiation exposure, bone marrow transplantation therapy or congenital anemia. Bone marrow transplantation (BMT) or HSC transplantation is a method widely used in clinical practice. By expanding HSC in vitro before transplantation will accelerate the hematopoiesis and increase the chance of success.

### SUMMARY

A CBP/-catenin antagonist is an agent that disrupts or inhibits, in vitro or in vivo, the interaction between the CBP and 3-catenin proteins. Examples of such CBP/-catenin antagonists can be found, for example, in U.S. Pat. Nos. 6,762,185; 7,531,320; 7,232,822; 7,563,825, and 7,598,253; and U.S. Patent Application Publication Nos. 20120296067; 20120088770; and 20110263607.

More effective compounds and methods for increasing hematopoiesis are also needed for the expansion of HSCs prior to differentiation. In some instances, the CBP/-catenin antagonists have been shown to promote hematopoietic stem cell (HSC) differentiation but not expansion. Without being bound by theory, it is believed that such compounds and methods, alone or in combination with existing CBP/-catenin antagonists, would improve therapies for increasing hematopoiesis in patients.

Thus, in one aspect, there is provided a compound of Formula I

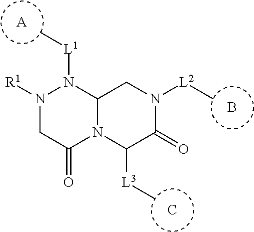

or a pharmaceutically acceptable salt thereof, wherein
R¹ is hydrogen or C₁-C₆ alkyl;
ring A is cyclohexyl or phenyl, optionally substituted;
ring B is aryl or a nitrogen-containing heteroaryl, optionally substituted;
ring C is hydroxyphenyl;
each L¹, L² and L³ is independently C₁-C₆ alkyl, —CONR²— or —CONR²—X—C₁-C₆ alkyl-, each optionally substituted; R² is hydrogen or C₁-C₆ alkyl; and
   X is a bond or a 5-6 membered heterocycle containing up to 3 ring heteroatoms. The compound can be combined with a carrier, such as a pharmaceutically acceptable carrier, for use in the methods as described herein.

In another aspect, a method is provided for increasing and/or supporting hematopoiesis in a subject in need thereof, comprising administering to the subject an effective amount of a compound of Formula I or a pharmaceutically acceptable salt thereof and/or a composition containing one or more compounds as described herein.

In yet another aspect, a method is provided for enhancing expansion of a hematopoietic stem cell or population (HSC), comprising contacting the HSC or population with an effective amount of a compound of Formula I or a pharmaceutically acceptable salt thereof and/or a composition containing one or more compounds as described herein. The contacting can be performed in vitro (e.g., including as part of an ex vivo method) or in vivo.

In still another aspect, a method is provided for inhibiting an interaction between a β-, and/or γ-catenin protein and a p-300 protein in a subject, cell or tissue having a β-, and/or γ-catenin protein and a p-300 protein, by contacting the cell, tissue or administering to the subject an effective amount of a compound of Formula I or a pharmaceutically acceptable salt thereof and/or a composition containing one or more compounds as described herein. In some aspects, the contacting is in vitro. In other aspects, the contacting is in vivo. In some aspects of the methods provided for inhibiting an interaction between a β-, and/or γ-catenin protein and a p-300 protein in a cell, a method for maintaining pluripotency of ES cells and iPS cells is provided.

In some embodiments of any of the methods described herein, the method further comprises administering to the subject or contacting the HSC cell or tissue with an effective amount of a CBP/β-catenin and/or γ-catenin antagonist that promotes hematopoietic stem cell (HSC) differentiation, wherein the antagonist is not a compound of Formula I or a pharmaceutically acceptable salt thereof. In one embodiment of the method, the antagonist is PRI-724 or ICG-001. In some embodiments, the method further comprises protection against fibrosis, which is a common chronic complication associated with radiation damage.

### BRIEF DESCRIPTION OF THE DRAWINGS

FIG. 1 provides a flow chart illustrating the screening strategy used in some of the Examples described herein.

FIGS. 2A-2D depict luciferase expression in both superTopflash and survivin reporter systems. FIGS. 2E-2H show that two close structural analogs (YH249 and YH250) both had IC50 values below 100 nM in the SuperTOPFLASH assay (FIG. 2F), yet did not inhibit the survivin/luciferase reporter up to 20 μM (FIG. 2G). These compounds therefore exhibit >200 fold selectivity for the p300/p-catenin interaction relative to the homologous CBP/p-catenin interaction (FIG. 2H).

FIG. 3 illustrates the results of “pull down” assays, in which decreased β-catenin was found in the presence of YH250, indicating that YH250 interfered with interactions between the β-catenin and p300 proteins.

FIG. 4A illustrates the procedure of purifying mice bone marrow Sca-1 positive cells for “pull down” assay (CO-IP). FIG. 4B shows CPB/catenin antagonist ICG-001 decreases γ-catenin interaction with CBP (lane B) while p300/catenin antagonist YH250 increases γ-catenin interaction with CBP (lane C) by interfering the interaction between γ-catenin and p300 protein.

FIG. 5A illustrates the procedure of purifying mice bone marrow Sca-1 positive cells for RNA isolation and subsequent RT-PCR analysis. P300 antagonist YH250 up-regulates Axin2 (FIG. 5B) and survivin (FIG. 5C) gene expression respectively.

FIG. 6A illustrates the procedure of isolating LTR-HSC (Long-Term-Repopulating HSC) from mice bone marrow for RNA sequencing. P300 antagonist YH250 up-regulates ID2 gene expression in LSKCD48-CD41-CD150+ cells (FIG. 6B).

FIG. 7A illustrates the procedure of isolating LTR-HSC and co-culture isolated LTR-HSC with supporting cells from GFP-transgenic mice. There are more un-differentiated CD48-CD202b+ cells (FIG. 7B) and less differentiated CD48+ cells (FIG. 7C) in YH250 treated cells.

FIG. 8A illustrates the procedures of colony forming cell (CFC) assay and colony forming units spleen day-12 (CFU-S₁₂) assay. Mice bone marrow cells were isolated and treated with either DMSO or YH250 (0.5 uM) for 4 hrs in serum free medium and then subjected to CFC or CFU-S₁₂ assay. In CFC assay, although the colony number in plate are similar between cells treated with DMSO or YH250, the colony size is larger from YH250 treated cells (FIG. 8B), and cell numbers recovered from the plate is higher from YH250 treated cells (FIG. 8C). FIG. 8D shows the results of CFU-S₁₂. FIG. 8E shows results of colony number and spleen weight.

FIG. 9 depicts the FACS analysis of bone marrow cells briefly incubated with DMSO or YH250 in vitro for 4 hrs and followed with 14 days culture in vitro.

FIG. 10 depicts the FACS analysis of bone marrow cells cultured with DMSO or YH250 for 3 days.

FIG. 11A depicts experimental procedures studying YH250 effects on cord blood (CB) cells in vivo and in vitro. FIG. 11B shows human cell engraftment in blood (left panel), bone marrow (middle panel) and human CD34+ cell engraftment in bone marrow (right panel).

FIG. 12A depicts cord blood CD34posit cells in different medium, growth factor and YH250 concentration. FIG. 12B shows the cells cultured 8 days in Stemp-span medium with 4 or 6 growth factors and different YH250 concentrations.

FIG. 13A depicts the FACS results from mice bone marrow cells with antibodies to human cell surface markers. FIG. 13B depicts FACS analysis of cultured cord blood CD34posit cells in the presence of mice carrier cells in FACS analysis.

Table 3 and Table 4 are summary of FACS analysis to cells cultured in different growth factor and YH250 concentrations.

FIG. 14A depicts the experimental procedure testing YH250 effect to cord blood cells in different in vitro culture medium. FIG. 14B shows the BMT results from in vitro treated cord blood cells by DMSO or YH250.

FIG. 15A depicts the experimental procedure studying YH250 function on bone marrow cell proliferation by BrdU incorporation assay. FIG. 15B shows the results of YH250 treatment, presented as BrdU-posit cell number in LSK or LSK34negt150posit population 24 or 48 hrs post YH250 administration.

FIG. 16A depicts the experimental procedure of testing the effects of multiple administration of YH250 in vivo to HSPC (Hematopoietic Stem Progenitor Cells). FIG. 16B shows the comparison of DMSO vs YH250 treated bone marrow cell in competitive repopulation assay. FIG. 16C shows BrdU-posit cell number in bone marrow HSPC population after 4 dose of YH250 treatment compared with DMSO treatment.

FIG. 17A depicts the experimental procedure testing YH250 effects to 7Gy irradiated mice bone marrow cell proliferation. FIG. 17B shows YH250 simulates irradiated animal bone marrow cells proliferation. FIG. 17C depicts the experimental procedure testing YH250 effect to 7Gy irradiated animal bone marrow cell cycling. FIG. 17D shows there are more bone marrow cells in S phase after YH250 treatment. FIG. 17E depicts experimental procedure testing YH250 effects to 7Gy irradiated animal HSC. FIG. 17F shows there are more Lin-CD48-CD150+ cells in YH250 treated 7Gy irradiated animals bone marrow.

FIG. 18A depicts the experimental procedure testing YH250 effects on 7Gy irradiated animal bone marrow hematopoiesis recovery. FIG. 18B shows that YH250 stimulates 7Gy irradiated animal HSC proliferation and accelerates hematopoiesis. FIG. 19A depicts the experimental procedure testing YH250 effects on 7Gy irradiated animals bone marrow progenitor cell recovery. FIG. 19B shows quicker recovery of hematopoiesis in YH 250 treated 7Gy irradiated animals.

FIG. 20A depicts experimental procedure testing YH250 effects on 7Gy irradiated animal peripheral blood count recovery. FIG. 20B compares the body weight changes of DMSO and YH250 treated 7Gy irradiated animals. FIG. 20C shows the peripheral blood count changes of YH250 treated vs DMSO treated 7Gy irradiated animals. FIG. 20D compares the blood count at nadir point between DMSO and YH250 treated animals post 7Gy radiation.

FIGS. 21A-21B depict the survival rate (21A) and body weight change (21B) of 9Gy (LD100/60) irradiated animals treated by DMSO and YH250. FIGS. 21C-21D depict the survival rate (21C) and body weight change (21D) of 8.5Gy (LD70/60) irradiated animals treated by DMSO and YH250. FIG. 21E shows that mice that received either YH250 or a combination of YH250 and ICG-001 showed significantly extended life-spans (p=0.0067 and 0.0033 for the YH 250 and YH250/ICG-001 groups versus the control group respectively).

FIG. 22A depicts the experimental procedure testing if YH250 exhausts HSC in rescuing irradiated animals. FIG. 22B shows YH250 does not affect HSC number and activity when administrated in animals with radiation induced bone marrow inhibition.

FIG. 23A depicts the experimental procedure studying YH250 in anti-apoptosis effect. FIG. 23B shows YH250 significantly decreased 5Gy radiation induced bone marrow cell apoptosis.

FIG. 24A depicts the experimental procedure studying if YH250 stimulates HSPC proliferation in chemotherapeutic drug (such as 5-FU) induced bone marrow inhibition. FIG. 24B shows there are more HPSC in YH250 treated animals received 5-FU treatment.

FIG. 25A depicts the experimental procedure studying if YH250 stimulates hematopoiesis under multiple administration of 5-FU. FIG. 25B shows the body weight changes after multiple 5-FU administration. FIG. 25C shows the survival rate after multiple 5-FU administration. FIG. 25D shows the change of blood count after multiple 5-FU administration. FIG. 25E shows the change of blood count at nadir points after each round of 5-FU administration.

FIG. 26A depicts the experimental procedure testing if YH250 exhausts HSC when used in multiple 5-FU induced bone marrow inhibition. FIG. 26B shows YH250 does not affect HSC number and activity in multiple 5-FU administration induced bone marrow inhibition.

FIG. 27 depicts both YH250 and YH249 increased HSPC numbers in in vitro culture. FIGS. 28A-28D show maintenance of ESC's pluripotency by YH249 and 250.

FIGS. 28A-28D show that p300/p-catenin Antagonists Maintained Pluripotency of Mouse and Human ES Cells and Human iPS Cells.

### DETAILED DESCRIPTION

### Definitions

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

As used in the specification and claims, the singular form “a”, “an” and “the” include plural references unless the context clearly dictates otherwise.

The term “about” when used before a numerical value indicates that the value may vary within a reasonable range, such as ±5%, ±1%, and ±0.2%.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention such as the biological activity of the claimed composition or method. “Consisting of” shall mean excluding more than trace amount of other ingredients and substantial method steps recited. Embodiments defined by each of these transition terms are within the scope of this invention.

As used herein, Cₘ-Cₙ, such as C₁-C₁₂, C₁-C₈, or C₁-C₆ when used before a group refers to that group containing m to n carbon atoms.

The term “alkoxy” refers to —O-alkyl.

The term “alkyl” refers to monovalent saturated aliphatic hydrocarbyl groups having from 1 to 12 carbon atoms (i.e., C₁-C₁₂ alkyl) or 1 to 6 carbon atoms (i.e., C₁-C₆ alkyl). This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom.

For example, and without limitation, the following is an aryl group:

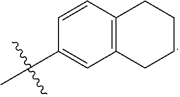 [SMILES: *c1ccc2c(c1)CCCC2]

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

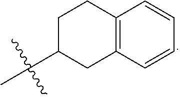 [SMILES: *C1CCc2ccccc2C1]

The term “halo” refers to F, Cl, Br, and/or I.

The term “heteroaryl” refers to a monovalent, aromatic mono-, bi-, or tricyclic ring having 2-16 ring carbon atoms and 1-8 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

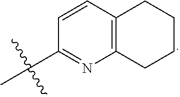 [SMILES: *c1ccc2c(n1)CCCC2]

The term “heterocyclyl” or heterocycle refers to a non-aromatic, mono-, bi-, or tricyclic ring containing 2-12 ring carbon atoms and 1-8 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that the ring is non-aromatic. Non-limiting examples of heterocyclyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

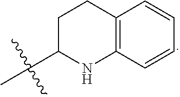 [SMILES: *C1CCc2ccccc2N1]

The term “oxo” refers to a C═O group, and to a substitution of 2 geminal hydrogen atoms with a C═O group.

The term “optionally substituted,” unless defined otherwise, refers to a substituted or unsubstituted group. The group may be substituted with one or more substituents, such as e.g., 1, 2, 3, 4 or 5 substituents. Preferably, the substituents are selected from the group consisting of oxo, halo, —CN, NO₂, —N₂+, —CO₂R¹⁰, —OR¹⁰, —SR¹⁰, —SOR¹⁰, —SO₂R¹⁰, —NR¹¹R¹², —CONR¹¹R¹², —SO₂NR¹¹R¹², C₁-C₆ alkyl, C₁-C₆ alkoxy, —CR¹⁰═C(R¹⁰)₂, —CCR¹⁰, C₃-C₁₀ cycloalkyl, C₃-C₁₀ heterocyclyl, C₆-C₁₂ aryl and C₂-C₁₂ heteroaryl, wherein each R¹⁰ independently is hydrogen or C₁-C₈ alkyl; C₃-C₁₂ cycloalkyl; C₃-C₁₀ heterocyclyl; C₆-C₁₂ aryl; or C₂-C₁₂ heteroaryl; wherein each alkyl, cycloalkyl, heterocyclyl, aryl, or heteroaryl is optionally substituted with 1-3 halo, 1-3 C₁-C₆ alkyl, 1-3 C₁-C₆ haloalkyl or 1-3 C₁-C₆ alkoxy groups. Preferably, the substituents are selected from the group consisting of chloro, fluoro, —OCH₃, methyl, ethyl, iso-propyl, cyclopropyl, vinyl, ethynyl, —CO₂H, —CO₂CH₃, —OCF₃, —CF₃ and —OCHF₂.

R¹¹ and R¹² independently is hydrogen; C₁-C₈ alkyl, optionally substituted with —CO₂H or an ester thereof, C₁-C₆ alkoxy, oxo, —CR¹³═C(R¹³)₂, —CCR, C₃-C₁₀ cycloalkyl, C₃-C₁₀ heterocyclyl, C₆-C₁₂ aryl, or C₂-C₁₂ heteroaryl, wherein each R¹³ independently is hydrogen or C₁-C₈ alkyl; C₃-C₁₂ cycloalkyl; C₃-C₁₀ heterocyclyl; C₆-C₁₂ aryl; or C₂-C₁₂ heteroaryl; wherein each cycloalkyl, heterocyclyl, aryl, or heteroaryl is optionally substituted with 1-3 alkyl groups or 1-3 halo groups, or R¹¹ and R¹² together with the nitrogen atom they are attached to form a 5-7 membered heterocycle.

The term “treatment” or “treating” means any treatment of a disease or disorder in a subject, such as a mammal, including:
preventing or protecting against the disease or disorder, that is, causing the clinical symptoms not to develop;
inhibiting the disease or disorder, that is, arresting or suppressing the development of clinical symptoms; and/or
relieving the disease or disorder that is, causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

As used herein, the term “condition” refers to a disease state for which the compounds, salts, compositions and methods provided herein are being used.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. A clone is a line of cells that is genetically identical to the originating cell; in this case, a stem cell.

The term “hematopoiesis” intends the formation and development of blood cells. In the embryo and fetus it takes place in a variety of sites including the liver, spleen, thymus, lymph nodes, and bone marrow; from birth throughout the rest of life it is mainly in the bone marrow with a small amount occurring in lymph nodes

As used herein, the term “hematopoietic stem cell” intends a pluripotent cell having the ability to differentiate into a cell of the hematopoietic lineage.

Bone marrow cells contain hematopoietic stem cells that give rise to all lineages, such as lymphoid, myeloid and erythroid lineages. Bone marrow includes stem cells as well as progenitor cells of the lymphoid (T and B cells), myeloid (e.g., granulocytes, macrophages, NK cell, etc.) and erythroid (red blood cells) and platelet lineages. Human hematopoietic stem and progenitor cells express CD34 on their surface while differentiated cells do not. Accordingly, the detection of CD34 can be used to distinguish differentiated from undifferentiated cells.

Hematopoietic precursor cells can be derived either from the patient (autologous transplant) or from a histocompatible donor (allogeneic donor). These cells can be isolated from bone marrow, peripheral blood or from umbilical cord blood. Bone marrow typically is aspirated from the iliac crest. Bone marrow is rich in CD34+ cells; typically 1 to 2% of marrow cells are precursor cells. Peripheral blood typically contains less than 1% CD34+ cells. Umbilical cord blood is very rich in early progenitor cells and may be used as a source of cells for hematopoietic cell transplant.

Separation of CD34+ cells (undifferentiated cells) from differentiated cells can be achieved by a number of different methods. The most widely used is a positive immunological selection based on binding of these cells to anti-CD34-antibodies immobilized on a solid support. Other selection methods include negative selection where all cells not expressing CD34 are isolated away from the CD34+ cells based on their expression of lineage specific cell surface antigens.

The number of progenitor cells that can be harvested at one time from either source is small and, in many cases, is not sufficient for a successful transplant. Several methods have been developed to expand bone marrow cells or progenitor cells obtained from blood apheresis or from umbilical cord blood in in vitro cultures. In vitro expansion of hematopoietic stem cells requires the addition of appropriate growth factors as well as certain growth conditions provided by so called stromal cells. Stromal cells provide physical support to hematopoietic progenitor cells as well as certain growth factors required for the increase of stem cell numbers.

A population of cells intends a collection of more than one cell that is identical (clonal) or non-identical in phenotype and/or genotype.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like which is susceptible to disease. In one embodiment, the mammals include horses, dogs, and cats. In another embodiment of the present invention, the human is an adolescent or infant under the age of eighteen years of age.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to achieve treatment dosages.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

As used herein, the “lineage” of a cell defines the heredity of the cell, i.e. its predecessors and progeny. The lineage of a cell places the cell within a hereditary scheme of development and differentiation.

As used herein, the term “differentiates or differentiated” defines a cell that takes on a more committed (“differentiated”) position within the lineage of a cell. “Dedifferentiated” defines a cell that reverts to a less committed position within the lineage of a cell.

As used herein, the term “p300/-catenin antagonist” is an agent that disrupts or inhibits, in vitro or in vivo, the interaction between the p300 and β-catenin proteins. In some embodiments, the p300/β-catenin antagonist promotes hematopoietic stem cell (HSC) expansion (i.e., proliferation, as the term is used herein). As used herein, the term CBP/β-catenin antagonist include the compounds of Formula I described herein. In some embodiments, the p300/β-catenin antagonist is selected from a compound of Table 1. In other embodiments, the p300/β-catenin antagonist is YH249 or YH250, having the structures:

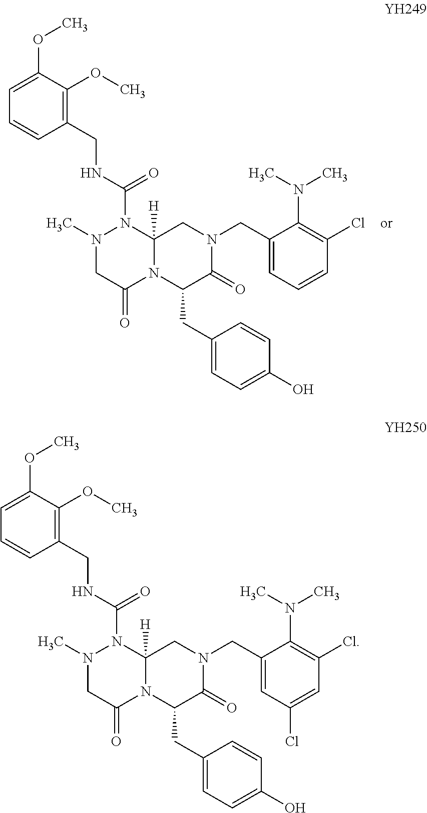 [SMILES: COc1cccc(CNC(=O)N2[C@H]3CN(Cc4cccc(Cl)c4N(C)C)C(=O)[C@H](Cc4ccc(O)cc4)N3C(=O)CN2C)c1OC ; COc1cccc(CNC(=O)N2[C@H]3CN(Cc4cc(Cl)cc(Cl)c4N(C)C)C(=O)[C@H](Cc4ccc(O)cc4)N3C(=O)CN2C)c1OC]

As used herein, the term “CBP/β-catenin antagonist” is an agent that disrupts or inhibits, in vitro or in vivo, the interaction between the CBP and β-catenin proteins. In some embodiments, the CBP/β-catenin antagonist promotes hematopoietic stem cell (HSC) differentiation. As used herein, the term CBP/β-catenin antagonist excludes the compounds of Formula I described herein. Exemplary CBP/β-catenin antagonists include those described in U.S. Pat. Nos. 6,762,185; 7,531,320; 7,232,822; 7,563,825, and 7,598,253; and U.S. Patent Application Publication Nos. 20120296067; 20120088770; and 20110263607. The compounds from these patents and patent applications, which are outside the scope of the compounds of Formula I described herein, are incorporated herein by reference as CBP/β-catenin antagonists. Exemplary CBP/β-catenin antagonists include PRI-724 under clinical development by Prism BioLab Co., Ltd (Japan) and ICG-001 having the structure:

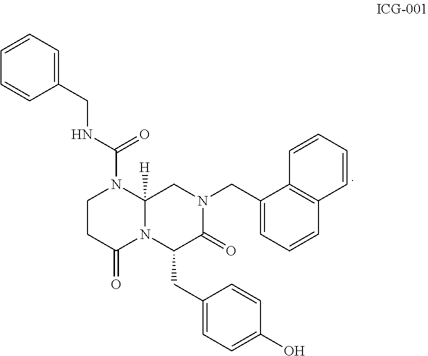 [SMILES: O=C1[C@H](Cc2ccc(O)cc2)N2C(=O)CCN(C(=O)NCc3ccccc3)[C@H]2CN1Cc1cccc2ccccc12]

The term “pharmaceutically acceptable” refers to safe and non-toxic use for in vivo, preferably, human administration.

The term “pharmaceutically acceptable salt” refers to a salt that is pharmaceutically acceptable.

The term “salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as caroboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

The term “carrier” as used herein, refers to relatively nontoxic chemical compounds or agents that facilitate the incorporation of a compound into cells, e.g., red blood cells, or tissues.

As used herein, a “prodrug” is a compound that, after administration, is metabolized or otherwise converted to an active or more active form with respect to at least one property.

To produce a prodrug, a pharmaceutically active compound can be modified chemically to render it less active or inactive, but the chemical modification is such that an active form of the compound is generated by metabolic or other biological processes. A prodrug may have, relative to the drug, altered metabolic stability or transport characteristics, fewer side effects or lower toxicity. For example, see the reference Nogrady, 1985, Medicinal Chemistry A Biochemical Approach, Oxford University Press, New York, pages 388-392. Prodrugs can also be prepared using compounds that are not drugs.

### I. COMPOUNDS

In one aspect, there is provided a compound of Formula I

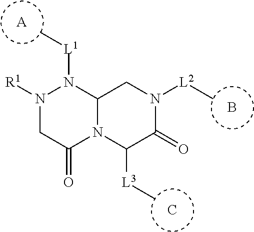

or a pharmaceutically acceptable salt thereof,
R¹ is hydrogen or C₁-C₆ alkyl;
ring A is cyclohexyl or phenyl, optionally substituted;
ring B is aryl or a nitrogen-containing heteroaryl, optionally substituted;
ring C is hydroxyphenyl;
each L¹, L² and L³ is independently C₁-C₆ alkyl, —CONR²— or —CONR²—X—C₁-C₆ alkyl-, each optionally substituted; R² is hydrogen or C₁-C₆ alkyl; and
X is a bond or a 5-6 membered heterocycle containing up to 3 ring heteroatoms.

In some embodiments, ring A is cyclohexyl or phenyl substituted with 1-4 groups selected from halo; C₁-C₆ alkyl, optionally substituted with 1-6 halo; C₁-C₆ alkoxy, optionally substituted with 1-6 halo; wherein two adjacent alkyl or alkoxy groups can join to form a 5- or 6-membered carbocyclic or heterocyclic ring. In other embodiments, ring A is

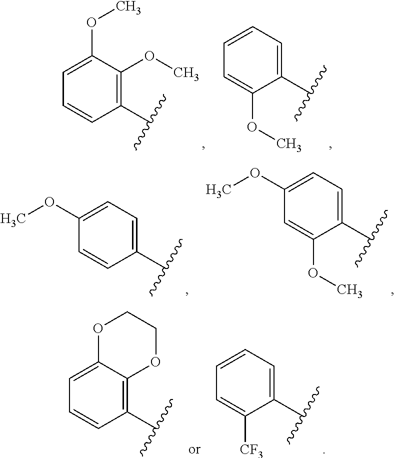

In some embodiments, ring B is phenyl, naphthyl, pyridyl, imidazolyl or quinolyl; wherein each is optionally substituted with 1-4 groups selected from halo; C₁-C₆ alkyl, optionally substituted with 1-6 halo; C₁-C₆ alkoxy, optionally substituted with 1-6 halo; NR³R4, wherein R³ and R⁴ are independently hydrogen or C₁-C₆ alkyl; or heterocyclyl, optionally substituted with C₁-C₆ alkyl; and two adjacent alkyl, alkoxy or NR³R⁴ groups can join to form a 5- or 6-membered carbocyclic or heterocyclic ring. In other embodiments, ring B is

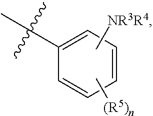

R⁵ is halo and n is 1, 2 or 3. In another embodiment, ring B is

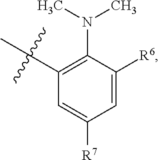

and R⁶ and R⁷ are independently halo.

In some embodiments, ring C is

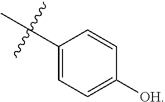 [SMILES: *c1ccc(O)cc1]

In some embodiments, L¹ is

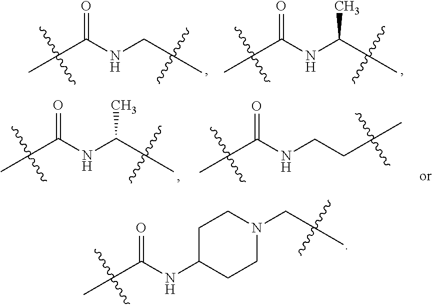

In other embodiments, L¹ is

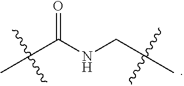 [SMILES: *CNC(*)=O]

In some embodiments, L² and L³ are —CH₂—.

In some embodiments, the compound is

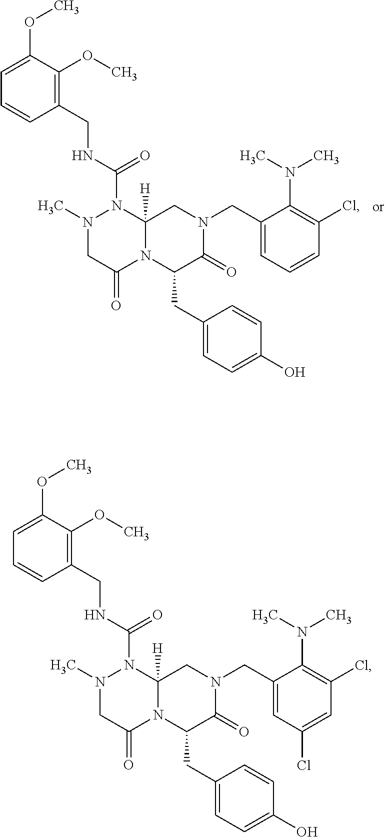 [SMILES: COc1cccc(CNC(=O)N2[C@H]3CN(Cc4cccc(Cl)c4N(C)C)C(=O)[C@H](Cc4ccc(O)cc4)N3C(=O)CN2C)c1OC ; COc1cccc(CNC(=O)N2[C@H]3CN(Cc4cc(Cl)cc(Cl)c4N(C)C)C(=O)[C@H](Cc4ccc(O)cc4)N3C(=O)CN2C)c1OC]

or a pharmaceutically acceptable salt thereof. In other embodiments, the compound is selected from the non-limiting group of representative compounds provided herein in Table 1.

TABLE 1
Representative compounds:

| No. | Compound. |
|---|---|
| YH-191 | 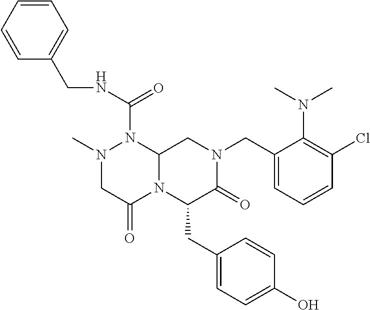 [SMILES: CN(C)c1c(Cl)cccc1CN1CC2N(C(=O)CN(C)N2C(=O)NCc2ccccc2)[C@@H](Cc2ccc(O)cc2)C1=O] |
| YH-192 | 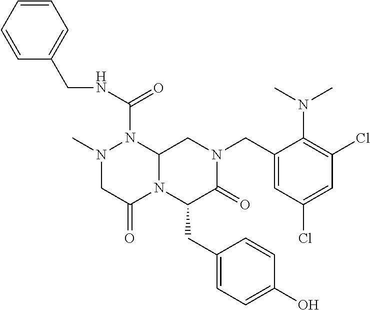 [SMILES: CN(C)c1c(Cl)cc(Cl)cc1CN1CC2N(C(=O)CN(C)N2C(=O)NCc2ccccc2)[C@@H](Cc2ccc(O)cc2)C1=O] |
| YH-193 | 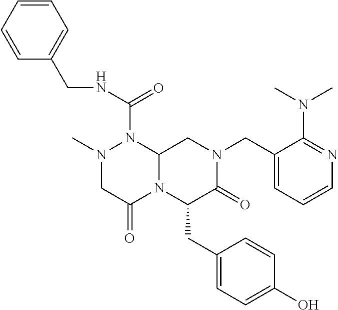 [SMILES: CN(C)c1ncccc1CN1CC2N(C(=O)CN(C)N2C(=O)NCc2ccccc2)[C@@H](Cc2ccc(O)cc2)C1=O] |
| YH-194 | 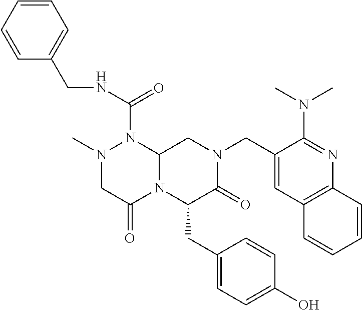 [SMILES: CN(C)c1nc2ccccc2cc1CN1CC2N(C(=O)CN(C)N2C(=O)NCc2ccccc2)[C@@H](Cc2ccc(O)cc2)C1=O] |
| YH-195 | 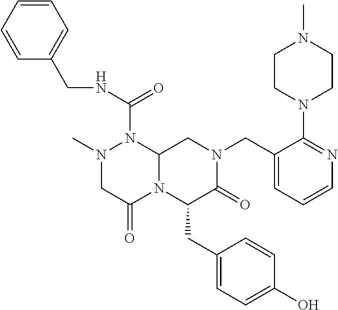 [SMILES: CN1CCN(c2ncccc2CN2CC3N(C(=O)CN(C)N3C(=O)NCc3ccccc3)[C@@H](Cc3ccc(O)cc3)C2=O)CC1] |
| YH-196 | 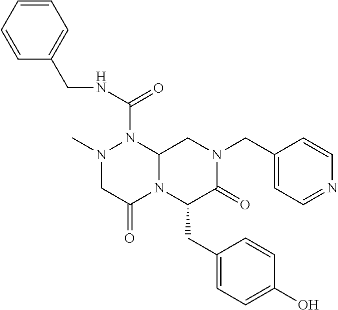 [SMILES: CN1CC(=O)N2C(CN(Cc3ccncc3)C(=O)[C@@H]2Cc2ccc(O)cc2)N1C(=O)NCc1ccccc1] |
| YH-197 | 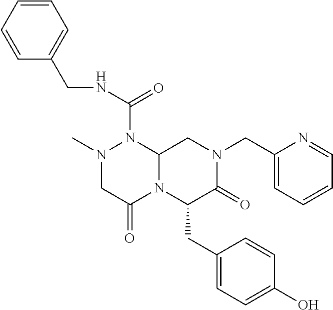 [SMILES: CN1CC(=O)N2C(CN(Cc3ccccn3)C(=O)[C@@H]2Cc2ccc(O)cc2)N1C(=O)NCc1ccccc1] |
| YH-198 | 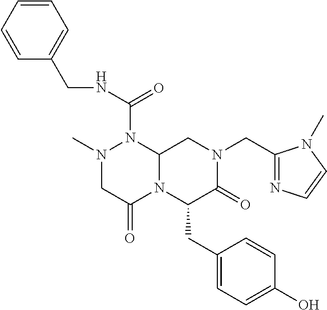 [SMILES: CN1CC(=O)N2C(CN(Cc3nccn3C)C(=O)[C@@H]2Cc2ccc(O)cc2)N1C(=O)NCc1ccccc1] |
| YH-199 | 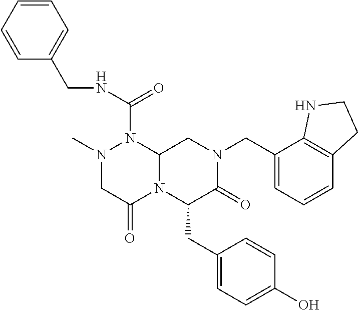 [SMILES: CN1CC(=O)N2C(CN(Cc3cccc4c3NCC4)C(=O)[C@@H]2Cc2ccc(O)cc2)N1C(=O)NCc1ccccc1] |
| YH-210 | 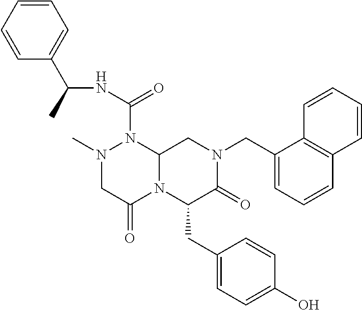 [SMILES: C[C@H](NC(=O)N1C2CN(Cc3cccc4ccccc34)C(=O)[C@H](Cc3ccc(O)cc3)N2C(=O)CN1C)c1ccccc1] |
| YH-211 | 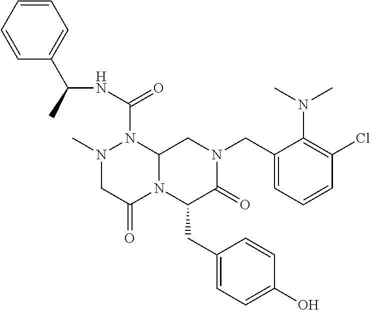 [SMILES: C[C@H](NC(=O)N1C2CN(Cc3cccc(Cl)c3N(C)C)C(=O)[C@H](Cc3ccc(O)cc3)N2C(=O)CN1C)c1ccccc1] |
| YH-212 | 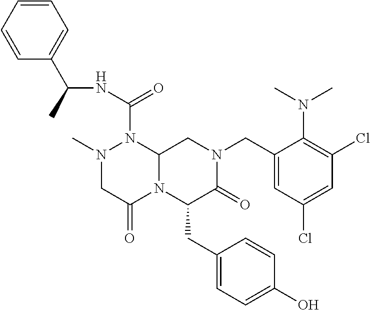 [SMILES: C[C@H](NC(=O)N1C2CN(Cc3cc(Cl)cc(Cl)c3N(C)C)C(=O)[C@H](Cc3ccc(O)cc3)N2C(=O)CN1C)c1ccccc1] |
| YH-213 | 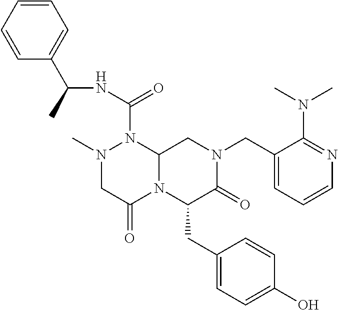 [SMILES: C[C@H](NC(=O)N1C2CN(Cc3cccnc3N(C)C)C(=O)[C@H](Cc3ccc(O)cc3)N2C(=O)CN1C)c1ccccc1] |
| YH-214 | 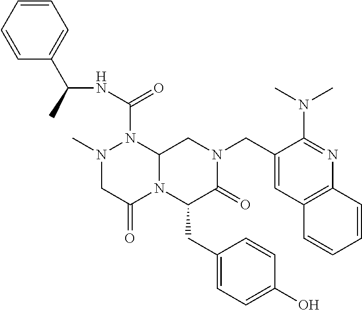 [SMILES: C[C@H](NC(=O)N1C2CN(Cc3cc4ccccc4nc3N(C)C)C(=O)[C@H](Cc3ccc(O)cc3)N2C(=O)CN1C)c1ccccc1] |
| YH-215 | 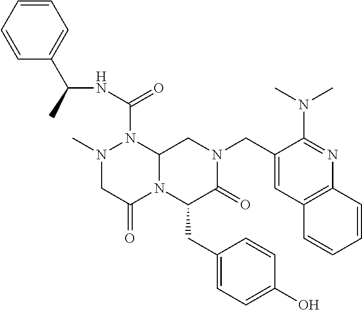 [SMILES: C[C@H](NC(=O)N1C2CN(Cc3cc4ccccc4nc3N(C)C)C(=O)[C@H](Cc3ccc(O)cc3)N2C(=O)CN1C)c1ccccc1] |
| YH-216 | 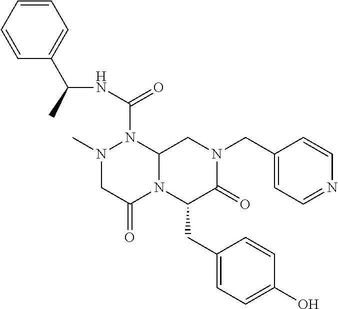 [SMILES: C[C@H](NC(=O)N1C2CN(Cc3ccncc3)C(=O)[C@H](Cc3ccc(O)cc3)N2C(=O)CN1C)c1ccccc1] |
| YH-217 | 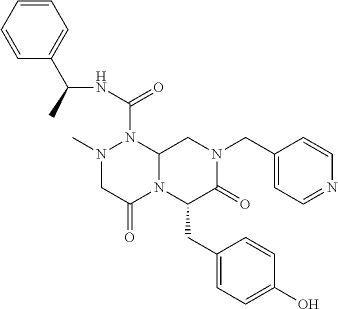 [SMILES: C[C@H](NC(=O)N1C2CN(Cc3ccncc3)C(=O)[C@H](Cc3ccc(O)cc3)N2C(=O)CN1C)c1ccccc1] |
| YH-218 | 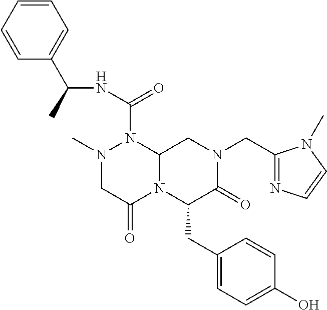 [SMILES: C[C@H](NC(=O)N1C2CN(Cc3nccn3C)C(=O)[C@H](Cc3ccc(O)cc3)N2C(=O)CN1C)c1ccccc1] |
| YH-219 | 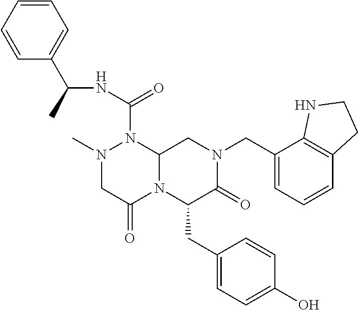 [SMILES: C[C@H](NC(=O)N1C2CN(Cc3cccc4c3NCC4)C(=O)[C@H](Cc3ccc(O)cc3)N2C(=O)CN1C)c1ccccc1] |
| YH-220 | 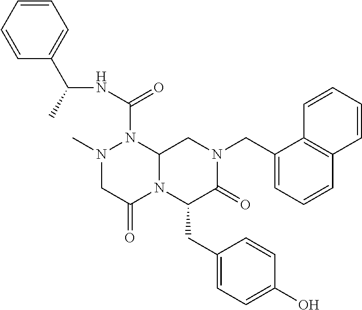 [SMILES: C[C@@H](NC(=O)N1C2CN(Cc3cccc4ccccc34)C(=O)[C@H](Cc3ccc(O)cc3)N2C(=O)CN1C)c1ccccc1] |
| YH-221 | 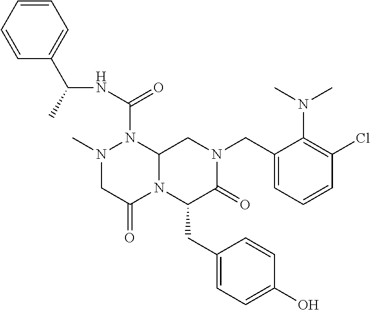 [SMILES: C[C@@H](NC(=O)N1C2CN(Cc3cccc(Cl)c3N(C)C)C(=O)[C@H](Cc3ccc(O)cc3)N2C(=O)CN1C)c1ccccc1] |
| YH-222 | 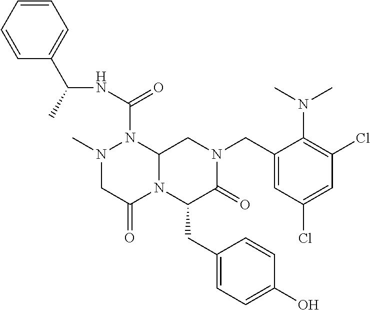 [SMILES: C[C@@H](NC(=O)N1C2CN(Cc3cc(Cl)cc(Cl)c3N(C)C)C(=O)[C@H](Cc3ccc(O)cc3)N2C(=O)CN1C)c1ccccc1] |
| YH-223 | 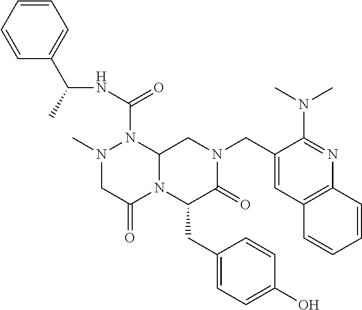 [SMILES: C[C@@H](NC(=O)N1C2CN(Cc3cc4ccccc4nc3N(C)C)C(=O)[C@H](Cc3ccc(O)cc3)N2C(=O)CN1C)c1ccccc1] |
| YH-224 | 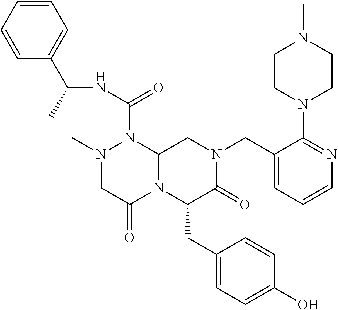 [SMILES: C[C@@H](NC(=O)N1C2CN(Cc3cccnc3N3CCN(C)CC3)C(=O)[C@H](Cc3ccc(O)cc3)N2C(=O)CN1C)c1ccccc1] |
| YH-225 | 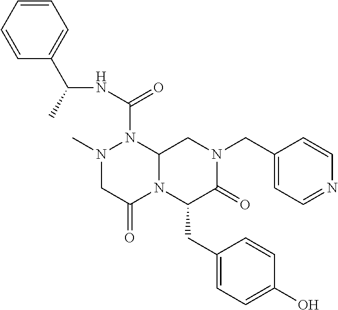 [SMILES: C[C@@H](NC(=O)N1C2CN(Cc3ccncc3)C(=O)[C@H](Cc3ccc(O)cc3)N2C(=O)CN1C)c1ccccc1] |
| YH-226 | 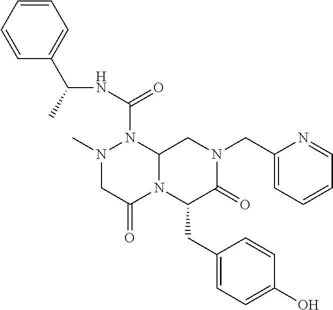 [SMILES: C[C@@H](NC(=O)N1C2CN(Cc3ccccn3)C(=O)[C@H](Cc3ccc(O)cc3)N2C(=O)CN1C)c1ccccc1] |
| YH-227 | 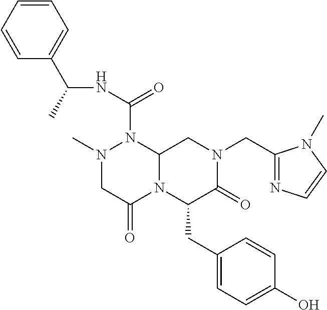 [SMILES: C[C@@H](NC(=O)N1C2CN(Cc3nccn3C)C(=O)[C@H](Cc3ccc(O)cc3)N2C(=O)CN1C)c1ccccc1] |
| YH-228 | 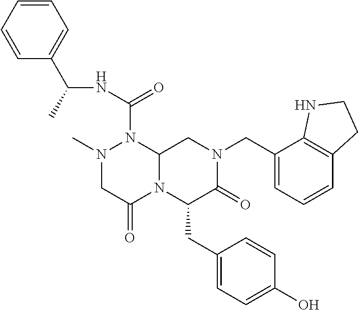 [SMILES: C[C@@H](NC(=O)N1C2CN(Cc3cccc4c3NCC4)C(=O)[C@H](Cc3ccc(O)cc3)N2C(=O)CN1C)c1ccccc1] |
| YH-229 | 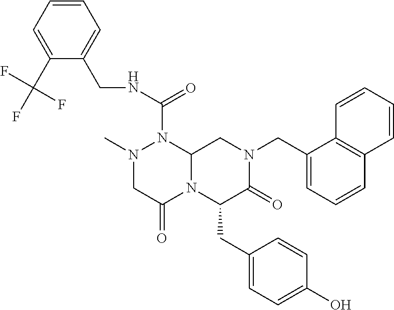 [SMILES: CN1CC(=O)N2C(CN(Cc3cccc4ccccc34)C(=O)[C@@H]2Cc2ccc(O)cc2)N1C(=O)NCc1ccccc1C(F)(F)F] |
| YH-230 | 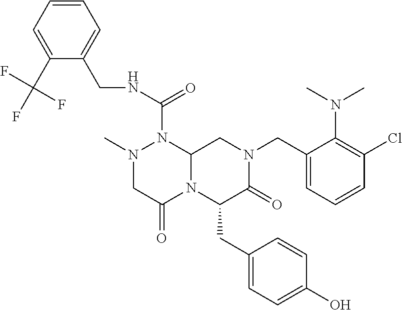 [SMILES: CN(C)c1c(Cl)cccc1CN1CC2N(C(=O)CN(C)N2C(=O)NCc2ccccc2C(F)(F)F)[C@@H](Cc2ccc(O)cc2)C1=O] |
| YH-231 | 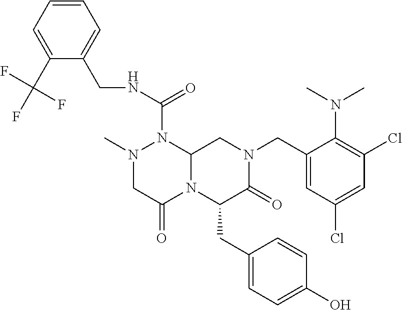 [SMILES: CN(C)c1c(Cl)cc(Cl)cc1CN1CC2N(C(=O)CN(C)N2C(=O)NCc2ccccc2C(F)(F)F)[C@@H](Cc2ccc(O)cc2)C1=O] |
| YH-232 | 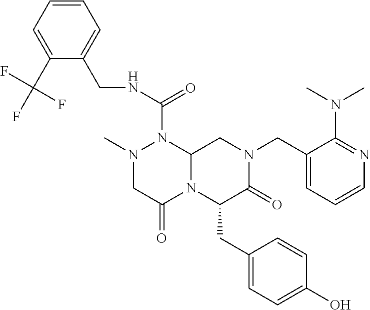 [SMILES: CN(C)c1ncccc1CN1CC2N(C(=O)CN(C)N2C(=O)NCc2ccccc2C(F)(F)F)[C@@H](Cc2ccc(O)cc2)C1=O] |
| YH-233 | 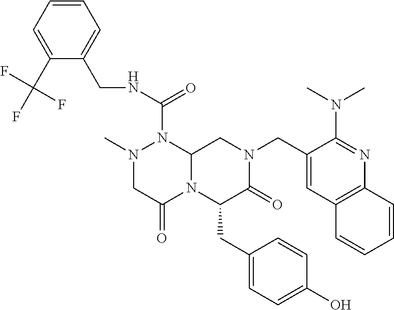 [SMILES: CN(C)c1nc2ccccc2cc1CN1CC2N(C(=O)CN(C)N2C(=O)NCc2ccccc2C(F)(F)F)[C@@H](Cc2ccc(O)cc2)C1=O] |
| YH-234 | 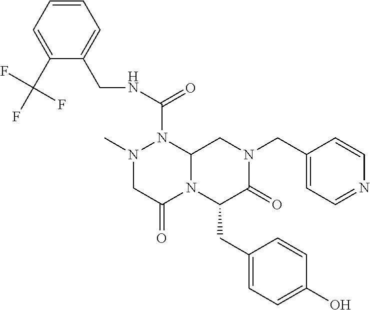 [SMILES: CN1CC(=O)N2C(CN(Cc3ccncc3)C(=O)[C@@H]2Cc2ccc(O)cc2)N1C(=O)NCc1ccccc1C(F)(F)F] |
| YH-235 | 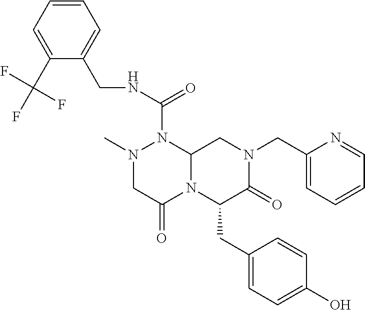 [SMILES: CN1CC(=O)N2C(CN(Cc3ccccn3)C(=O)[C@@H]2Cc2ccc(O)cc2)N1C(=O)NCc1ccccc1C(F)(F)F] |
| YH-236 | 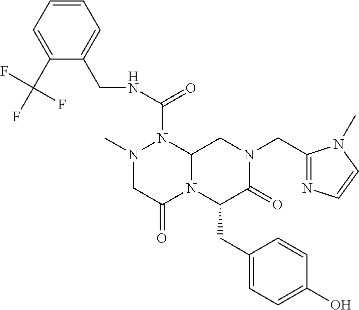 [SMILES: CN1CC(=O)N2C(CN(Cc3nccn3C)C(=O)[C@@H]2Cc2ccc(O)cc2)N1C(=O)NCc1ccccc1C(F)(F)F] |
| YH-237 | 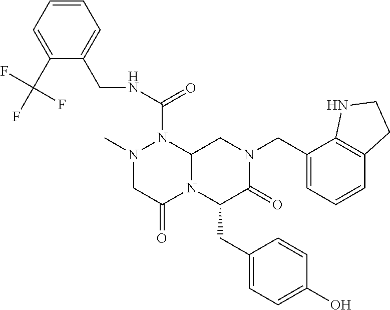 [SMILES: CN1CC(=O)N2C(CN(Cc3cccc4c3NCC4)C(=O)[C@@H]2Cc2ccc(O)cc2)N1C(=O)NCc1ccccc1C(F)(F)F] |
| YH-238 | 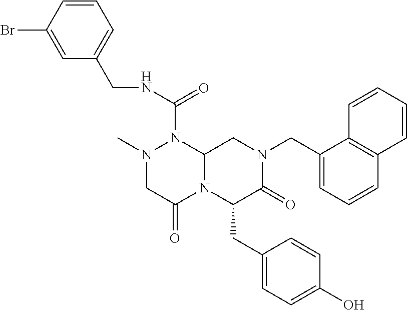 [SMILES: CN1CC(=O)N2C(CN(Cc3cccc4ccccc34)C(=O)[C@@H]2Cc2ccc(O)cc2)N1C(=O)NCc1cccc(Br)c1] |
| YH-200 | 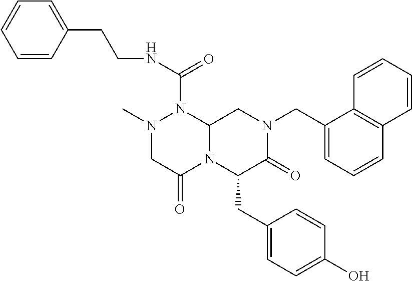 [SMILES: CN1CC(=O)N2C(CN(Cc3cccc4ccccc34)C(=O)[C@@H]2Cc2ccc(O)cc2)N1C(=O)NCCc1ccccc1] |
| YH-201 | 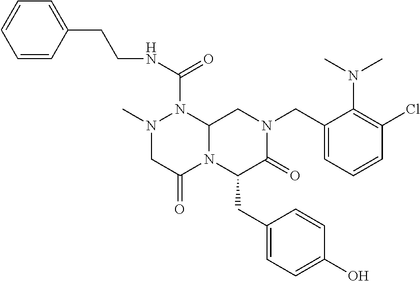 [SMILES: CN(C)c1c(Cl)cccc1CN1CC2N(C(=O)CN(C)N2C(=O)NCCc2ccccc2)[C@@H](Cc2ccc(O)cc2)C1=O] |
| YH-202 | 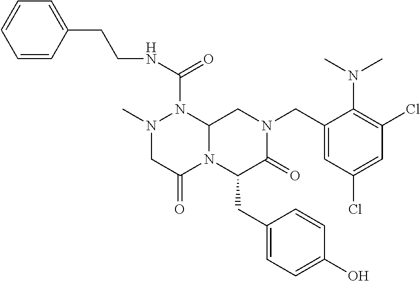 [SMILES: CN(C)c1c(Cl)cc(Cl)cc1CN1CC2N(C(=O)CN(C)N2C(=O)NCCc2ccccc2)[C@@H](Cc2ccc(O)cc2)C1=O] |
| YH-203 | 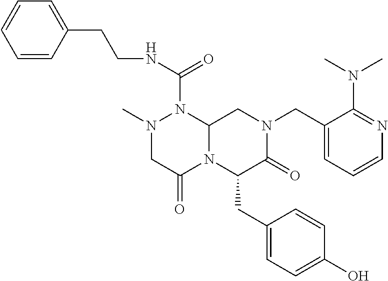 [SMILES: CN(C)c1ncccc1CN1CC2N(C(=O)CN(C)N2C(=O)NCCc2ccccc2)[C@@H](Cc2ccc(O)cc2)C1=O] |
| YH-204 | 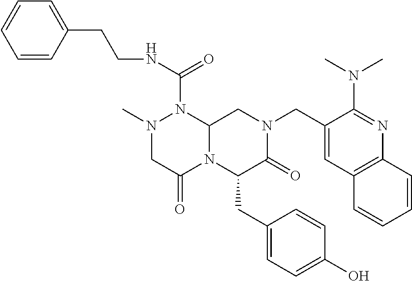 [SMILES: CN(C)c1nc2ccccc2cc1CN1CC2N(C(=O)CN(C)N2C(=O)NCCc2ccccc2)[C@@H](Cc2ccc(O)cc2)C1=O] |
| YH-205 | 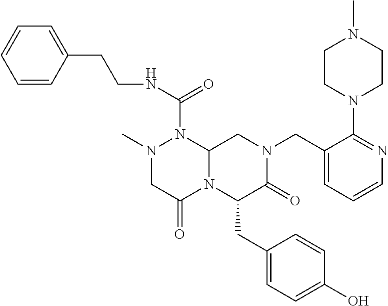 [SMILES: CN1CCN(c2ncccc2CN2CC3N(C(=O)CN(C)N3C(=O)NCCc3ccccc3)[C@@H](Cc3ccc(O)cc3)C2=O)CC1] |
| YH-206 | 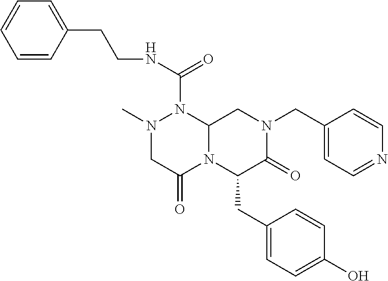 [SMILES: CN1CC(=O)N2C(CN(Cc3ccncc3)C(=O)[C@@H]2Cc2ccc(O)cc2)N1C(=O)NCCc1ccccc1] |
| YH-207 | 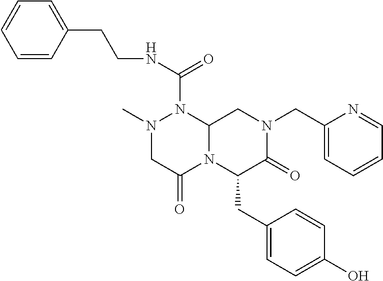 [SMILES: CN1CC(=O)N2C(CN(Cc3ccccn3)C(=O)[C@@H]2Cc2ccc(O)cc2)N1C(=O)NCCc1ccccc1] |
| YH-208 | 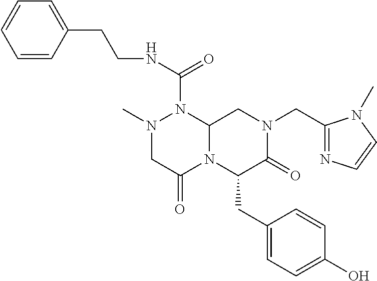 [SMILES: CN1CC(=O)N2C(CN(Cc3nccn3C)C(=O)[C@@H]2Cc2ccc(O)cc2)N1C(=O)NCCc1ccccc1] |
| YH-209 | 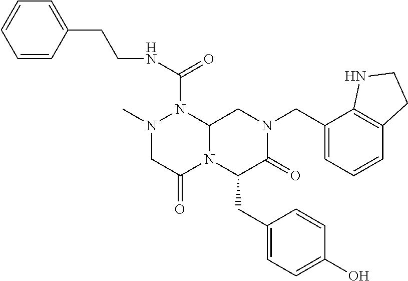 [SMILES: CN1CC(=O)N2C(CN(Cc3cccc4c3NCC4)C(=O)[C@@H]2Cc2ccc(O)cc2)N1C(=O)NCCc1ccccc1] |
| YH-239 | 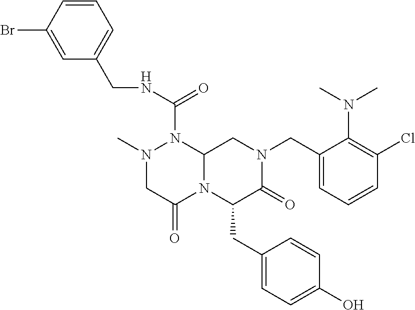 [SMILES: CN(C)c1c(Cl)cccc1CN1CC2N(C(=O)CN(C)N2C(=O)NCc2cccc(Br)c2)[C@@H](Cc2ccc(O)cc2)C1=O] |
| YH-240 | 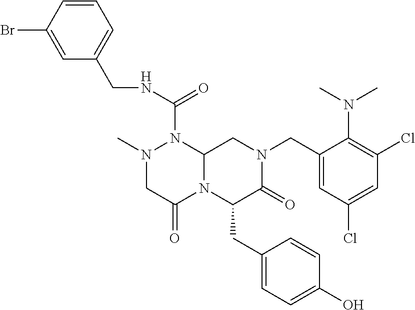 [SMILES: CN(C)c1c(Cl)cc(Cl)cc1CN1CC2N(C(=O)CN(C)N2C(=O)NCc2cccc(Br)c2)[C@@H](Cc2ccc(O)cc2)C1=O] |
| YH-241 | 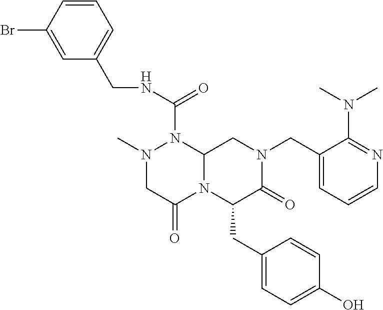 [SMILES: CN(C)c1ncccc1CN1CC2N(C(=O)CN(C)N2C(=O)NCc2cccc(Br)c2)[C@@H](Cc2ccc(O)cc2)C1=O] |
| YH-242 | 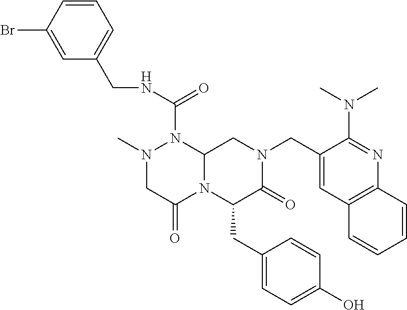 [SMILES: CN(C)c1nc2ccccc2cc1CN1CC2N(C(=O)CN(C)N2C(=O)NCc2cccc(Br)c2)[C@@H](Cc2ccc(O)cc2)C1=O] |
| YH-243 | 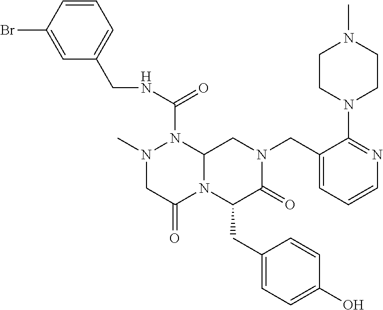 [SMILES: CN1CCN(c2ncccc2CN2CC3N(C(=O)CN(C)N3C(=O)NCc3cccc(Br)c3)[C@@H](Cc3ccc(O)cc3)C2=O)CC1] |
| YH-244 | 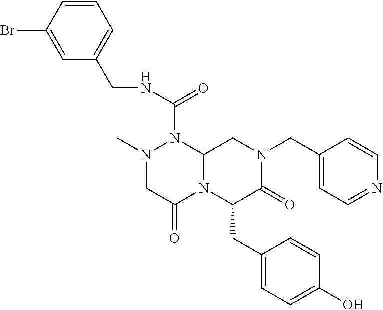 [SMILES: CN1CC(=O)N2C(CN(Cc3ccncc3)C(=O)[C@@H]2Cc2ccc(O)cc2)N1C(=O)NCc1cccc(Br)c1] |
| YH-245 | 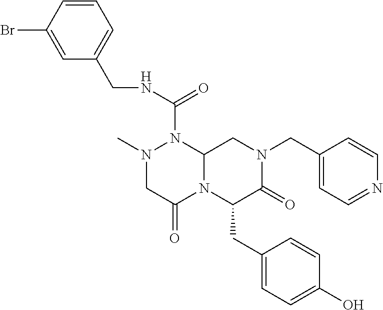 [SMILES: CN1CC(=O)N2C(CN(Cc3ccncc3)C(=O)[C@@H]2Cc2ccc(O)cc2)N1C(=O)NCc1cccc(Br)c1] |
| YH-246 | 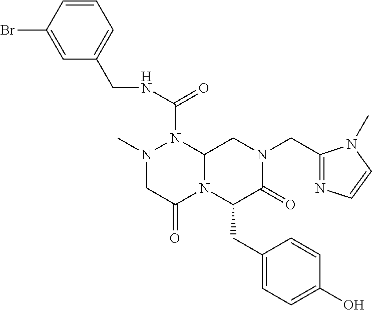 [SMILES: CN1CC(=O)N2C(CN(Cc3nccn3C)C(=O)[C@@H]2Cc2ccc(O)cc2)N1C(=O)NCc1cccc(Br)c1] |
| YH-247 | 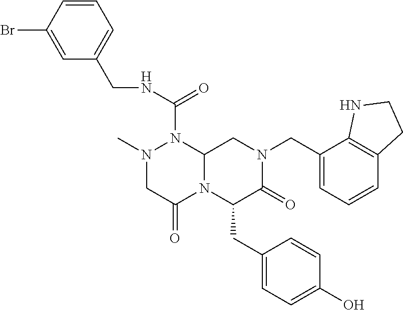 [SMILES: CN1CC(=O)N2C(CN(Cc3cccc4c3NCC4)C(=O)[C@@H]2Cc2ccc(O)cc2)N1C(=O)NCc1cccc(Br)c1] |
| YH-248 | 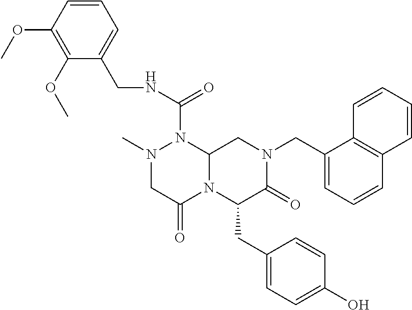 [SMILES: COc1cccc(CNC(=O)N2C3CN(Cc4cccc5ccccc45)C(=O)[C@H](Cc4ccc(O)cc4)N3C(=O)CN2C)c1OC] |
| YH-249 | 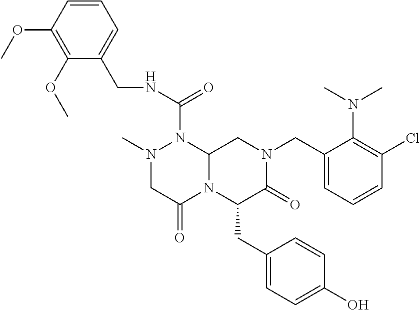 [SMILES: COc1cccc(CNC(=O)N2C3CN(Cc4cccc(Cl)c4N(C)C)C(=O)[C@H](Cc4ccc(O)cc4)N3C(=O)CN2C)c1OC] |
| YH-250 | 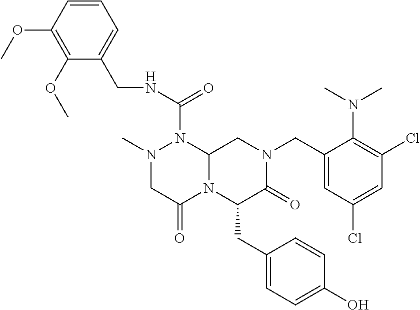 [SMILES: COc1cccc(CNC(=O)N2C3CN(Cc4cc(Cl)cc(Cl)c4N(C)C)C(=O)[C@H](Cc4ccc(O)cc4)N3C(=O)CN2C)c1OC] |
| YH-251 | 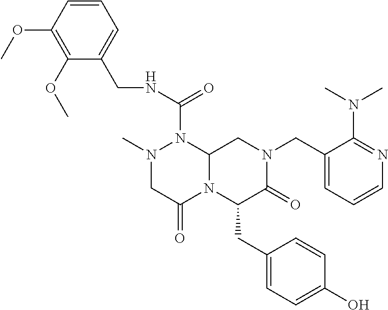 |
| YH-252 | 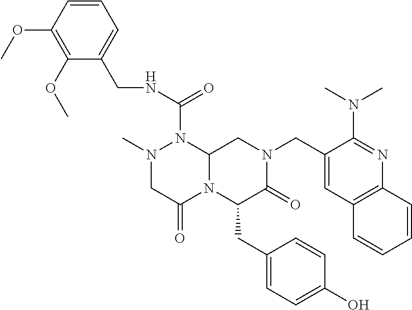 [SMILES: COc1cccc(CNC(=O)N2C3CN(Cc4cc5ccccc5nc4N(C)C)C(=O)[C@H](Cc4ccc(O)cc4)N3C(=O)CN2C)c1OC] |
| YH-253 | 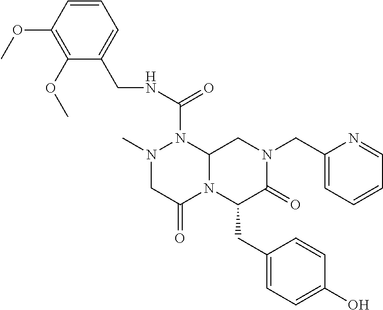 [SMILES: COc1cccc(CNC(=O)N2C3CN(Cc4ccccn4)C(=O)[C@H](Cc4ccc(O)cc4)N3C(=O)CN2C)c1OC] |
| YH-254 | 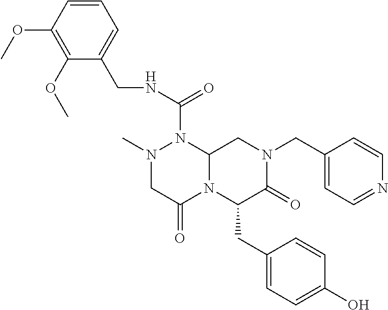 [SMILES: COc1cccc(CNC(=O)N2C3CN(Cc4ccncc4)C(=O)[C@H](Cc4ccc(O)cc4)N3C(=O)CN2C)c1OC] |
| YH-255 | 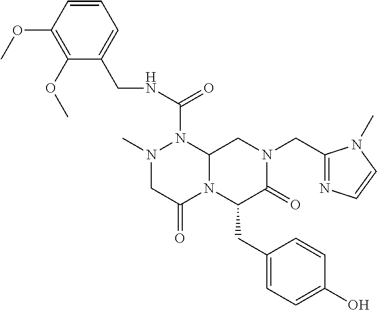 [SMILES: COc1cccc(CNC(=O)N2C3CN(Cc4nccn4C)C(=O)[C@H](Cc4ccc(O)cc4)N3C(=O)CN2C)c1OC] |
| YH-256 | 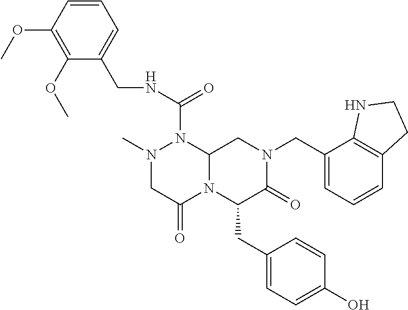 [SMILES: COc1cccc(CNC(=O)N2C3CN(Cc4cccc5c4NCC5)C(=O)[C@H](Cc4ccc(O)cc4)N3C(=O)CN2C)c1OC] |
| YH-257 | 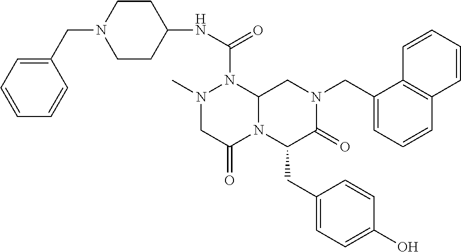 [SMILES: CN1CC(=O)N2C(CN(Cc3cccc4ccccc34)C(=O)[C@@H]2Cc2ccc(O)cc2)N1C(=O)NC1CCN(Cc2ccccc2)CC1] |
| YH-258 | 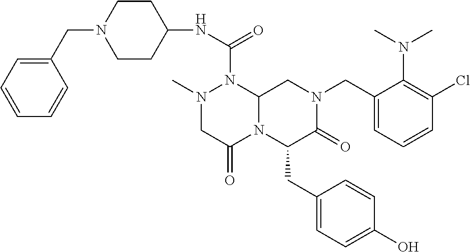 [SMILES: CN(C)c1c(Cl)cccc1CN1CC2N(C(=O)CN(C)N2C(=O)NC2CCN(Cc3ccccc3)CC2)[C@@H](Cc2ccc(O)cc2)C1=O] |
| YH-259 | 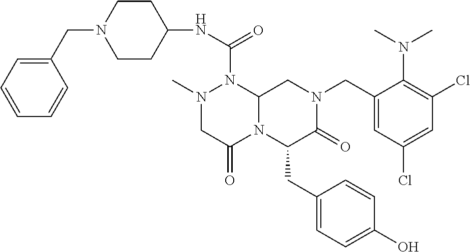 [SMILES: CN(C)c1c(Cl)cc(Cl)cc1CN1CC2N(C(=O)CN(C)N2C(=O)NC2CCN(Cc3ccccc3)CC2)[C@@H](Cc2ccc(O)cc2)C1=O] |
| YH-260 | 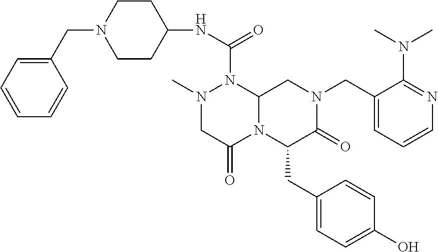 [SMILES: CN(C)c1ncccc1CN1CC2N(C(=O)CN(C)N2C(=O)NC2CCN(Cc3ccccc3)CC2)[C@@H](Cc2ccc(O)cc2)C1=O] |
| YH-261 | 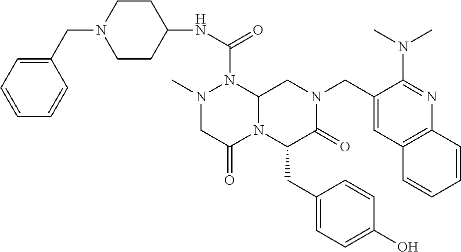 [SMILES: CN(C)c1nc2ccccc2cc1CN1CC2N(C(=O)CN(C)N2C(=O)NC2CCN(Cc3ccccc3)CC2)[C@@H](Cc2ccc(O)cc2)C1=O] |
| YH-262 | 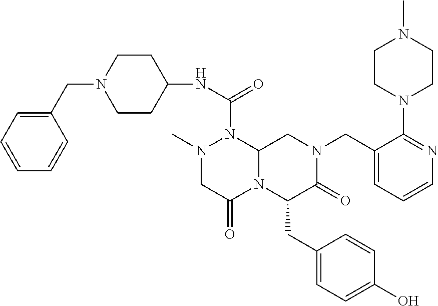 [SMILES: CN1CCN(c2ncccc2CN2CC3N(C(=O)CN(C)N3C(=O)NC3CCN(Cc4ccccc4)CC3)[C@@H](Cc3ccc(O)cc3)C2=O)CC1] |
| YH-263 | 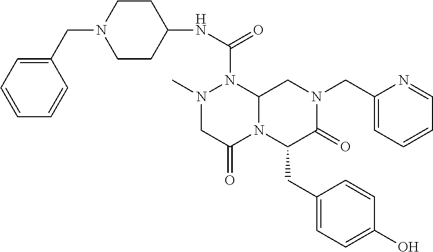 [SMILES: CN1CC(=O)N2C(CN(Cc3ccccn3)C(=O)[C@@H]2Cc2ccc(O)cc2)N1C(=O)NC1CCN(Cc2ccccc2)CC1] |
| YH-264 | 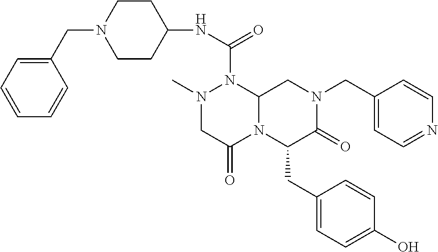 [SMILES: CN1CC(=O)N2C(CN(Cc3ccncc3)C(=O)[C@@H]2Cc2ccc(O)cc2)N1C(=O)NC1CCN(Cc2ccccc2)CC1] |
| YH-265 | 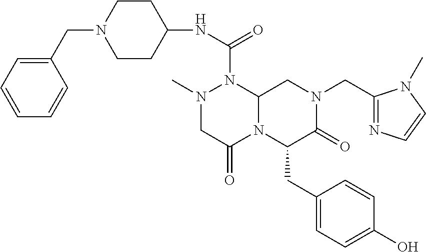 [SMILES: CN1CC(=O)N2C(CN(Cc3nccn3C)C(=O)[C@@H]2Cc2ccc(O)cc2)N1C(=O)NC1CCN(Cc2ccccc2)CC1] |
| YH-266 | 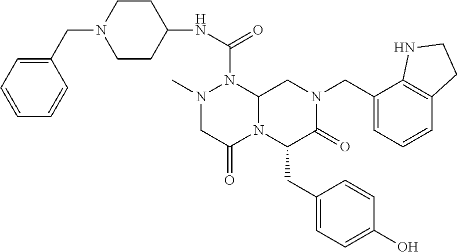 [SMILES: CN1CC(=O)N2C(CN(Cc3cccc4c3NCC4)C(=O)[C@@H]2Cc2ccc(O)cc2)N1C(=O)NC1CCN(Cc2ccccc2)CC1] |
| YH-267 | 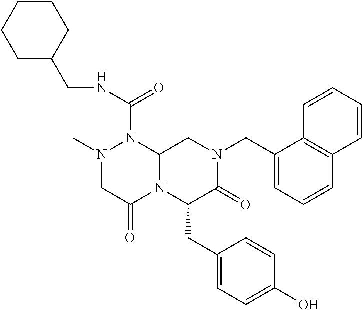 [SMILES: CN1CC(=O)N2C(CN(Cc3cccc4ccccc34)C(=O)[C@@H]2Cc2ccc(O)cc2)N1C(=O)NCC1CCCCC1] |
| YH-269 | 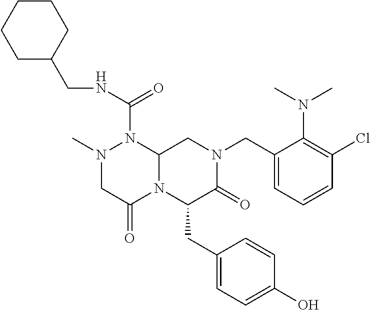 [SMILES: CN(C)c1c(Cl)cccc1CN1CC2N(C(=O)CN(C)N2C(=O)NCC2CCCCC2)[C@@H](Cc2ccc(O)cc2)C1=O] |
| YH-268 | 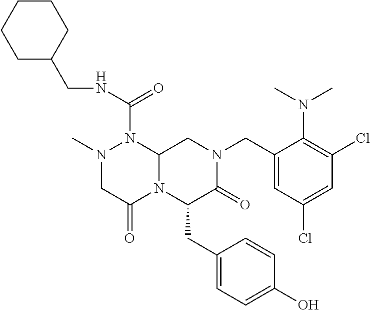 [SMILES: CN(C)c1c(Cl)cc(Cl)cc1CN1CC2N(C(=O)CN(C)N2C(=O)NCC2CCCCC2)[C@@H](Cc2ccc(O)cc2)C1=O] |
| YH-270 | 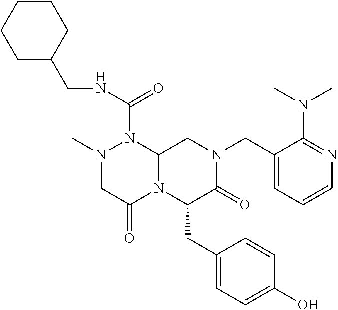 [SMILES: CN(C)c1ncccc1CN1CC2N(C(=O)CN(C)N2C(=O)NCC2CCCCC2)[C@@H](Cc2ccc(O)cc2)C1=O] |
| YH-271 | 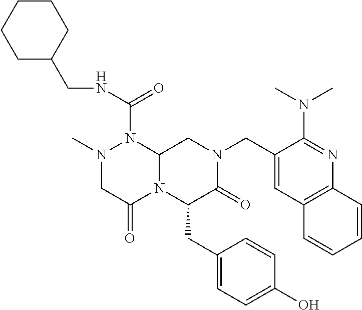 [SMILES: CN(C)c1nc2ccccc2cc1CN1CC2N(C(=O)CN(C)N2C(=O)NCC2CCCCC2)[C@@H](Cc2ccc(O)cc2)C1=O] |
| YH-272 | 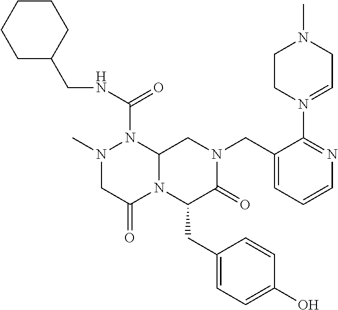 |
| YH-273 | 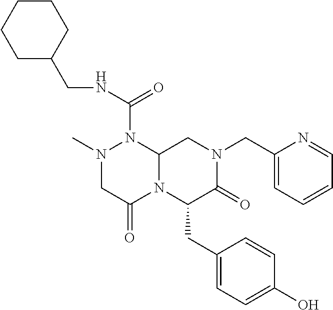 [SMILES: CN1CC(=O)N2C(CN(Cc3ccccn3)C(=O)[C@@H]2Cc2ccc(O)cc2)N1C(=O)NCC1CCCCC1] |
| YH-274 | 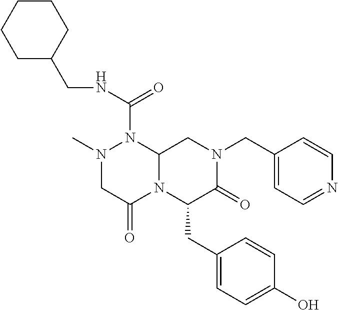 [SMILES: CN1CC(=O)N2C(CN(Cc3ccncc3)C(=O)[C@@H]2Cc2ccc(O)cc2)N1C(=O)NCC1CCCCC1] |
| YH-275 | 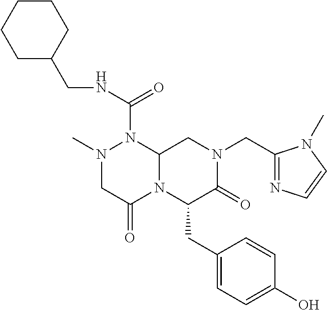 [SMILES: CN1CC(=O)N2C(CN(Cc3nccn3C)C(=O)[C@@H]2Cc2ccc(O)cc2)N1C(=O)NCC1CCCCC1] |
| YH-276 | 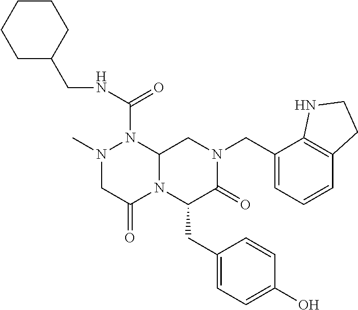 [SMILES: CN1CC(=O)N2C(CN(Cc3cccc4c3NCC4)C(=O)[C@@H]2Cc2ccc(O)cc2)N1C(=O)NCC1CCCCC1] |

### II. PHARMACEUTICAL COMPOSITIONS

In another aspect, provided herein is pharmaceutical composition comprising any of the compounds of Formula I as described herein and a pharmaceutically acceptable carrier and/or excipient. Pharmaceutically acceptable carriers or excipients are known in the art, for example, as described in Remington's Pharmaceutical Sciences, Mack Publishing Co., (A. R. Gennaro Ed. 1985). In some embodiments, a compound of Formula I or a pharmaceutically acceptable salt, hydrate, and/or polymorph thereof may be administered in compositions, such as solutions, suspensions, tablets, capsules, lozenges or elixirs for oral administration, suppositories, sterile solutions or suspensions or injectable administration, and the like, or incorporated into shaped articles.

The dose and method of administration of the compound of Formula I will vary from subject to subject and be dependent upon such factors as the type of mammal being treated, its sex, weight, diet, concurrent medication, overall clinical condition, the specific use for which A compound of Formula I is employed, and other factors which those skilled in the medical arts will recognize. In some embodiments, about 0.5 to 500 mg of A compound of Formula I or a pharmaceutically acceptable salt, hydrate, and/or polymorph thereof is combined with a physiologically acceptable vehicle, carrier, excipient, binder, preservative, stabilizer, dye, and/or flavor etc., as called for by accepted pharmaceutical practice.

In some embodiments, the compound of Formula I or a pharmaceutically acceptable salt, hydrate, and/or polymorph thereof, is administered orally in a delayed release enteric coated (EC) tablet. In some embodiments, the compound of Formula I or a pharmaceutically acceptable salt, hydrate, and/or polymorph thereof, is administered orally in an immediate release (IR) capsule. In some embodiments, the compound of Formula I or a pharmaceutically acceptable salt, hydrate, and/or polymorph thereof, is administered orally in a composition comprising dextrose monohydrate, croscarmellose sodium and magnesium stearate. In some embodiments, the composition is granulated and filled into a hard gelatin capsule.

In some embodiments, the compound of Formula I or a pharmaceutically acceptable salt, hydrate, and/or polymorph thereof is administered from about 0.001 mg/kg to about 1000 mg/kg, preferably from about 0.01 mg/kg to about 100 mg/kg, and more preferably from about 0.10 mg/kg to about 20 mg/kg.

In some embodiments, the compound of Formula I or a pharmaceutically acceptable salt, hydrate, and/or polymorph thereof is administered to a patient in a daily dosage of between about 10 mg and about 20 mg, between about 25 mg and 35 mg, or between about 40 mg and about 120 mg. In some embodiments, the compound of Formula I or a pharmaceutically acceptable salt, hydrate, and/or polymorph thereof, is administered in a daily dosage of about 40, 50, 60, 70, 80, 90, 100, 110 or 120 mg. In some embodiments, the compound of Formula I or a pharmaceutically acceptable salt, hydrate, and/or polymorph thereof is administered in a daily dosage of about 10, 15, 20, 25, 30, 40, 60, 80 or 90 mg. In some embodiments, the compound of Formula I or a pharmaceutically acceptable salt, hydrate, and/or polymorph thereof, is administered in a daily dosage of about 40, 60 or 80 mg. In some embodiments, the compound of Formula I or a pharmaceutically acceptable salt, hydrate, and/or polymorph thereof, is administered once, twice or three times a day, preferably once or twice daily.

The compound of Formula I may also be administered in a composition comprising one or more pharmaceutically acceptable carriers or excipients, and/or an effective amount of another therapeutic agent such as a granulocyte-colony stimulating factor (G-CSF) and/or erythropoietin (EPO) or an equivalent thereof.

### III. METHODS OF TREATMENT

Without being bound by theory, it is contemplated that compounds, such as those of Formula I described herein, can block or inhibit the interaction between a β-, and/or γ-catenin and p300 and/or promote stem/progenitor cell proliferation. As used herein, the term “block or inhibit the interaction” intends a diminution or reduction in the binding between a β-, and/or γ-catenin and p300 in a cell or tissue having such proteins. The methods also will enhance the interaction between CBP with a β-, and/or γ-catenin. Methods of determining the binding efficiency or interaction between a β-, and/or γ-catenin and p300 or CBP are known in the art and described herein. This method can be performed in vitro in a cell culture or tissue system or in vivo, by administering an effective amount of the compound to a subject. When performed in vitro, they provide a convenient in vitro screen to determine efficacy or dose of the compound or composition containing same use alone or in combination with another active agent. When performed in vivo, they can have therapeutic benefit or be used in an appropriate animal model for pre-clinical or investigational use. As noted herein, the p300/P3-, and/or γ-catenin antagonists described herein promote stem/progenitor cell division in symmetrical non-differentiated patterns and thereby expand stem/progenitor cells.

Bone marrow, stem cell and cord blood stem cell transplantations are performed for many clinical indications such as during the treatment of different types of cancers and aplastic anemia. The dose of the transplanted stem cell is often times an important element of such procedures. Cord blood banking can only be expected to provide modest doses for future stem cell transplantations. In vitro stem cell expansions of the rare stem cells or populations would be far more flexible and convenient and ensure greater chance of clinical success. The methods of this disclosure can be used on any populations known or believed to contain hematopoietic stem cells. The cells or tissue can be isolated from a subject or retrieved from a bank. Thus, the cells or tissue can be primary or cultured cells or tissue.

As shown in the examples below, YH250 and YH249 were found to block the interaction between a β-, and/or γ-catenin and p300 and enhance the interaction between a β-, and/or γ-catenin and CBP. These compounds also resulted in the expansion of hematopoietic stem cells. Thus, it is shown herein that these β-, and/or γ-catenin/p300 antagonists and similar such compounds of Formula I can be used in methods for the expansion of HSCs. One of skill in the art can determine if stem cell expansion has occurred by screening the treated cells or tissue for the number of cells having the appropriate cell markers, e.g., markers that indicate a more immature phenotype or lineage cell markers

Non-limiting exemplary human hematopoietic stem cell markers include cells that are lineage negative, CD34 positive and CD38 negative. Lineage cell markers include T cell markers such as CD3, CD4 and CD8; B cell markers such as CD19 and CD20; and myeloid cell markers such as CD14, CD16 and CD11b.

Non-limiting exemplary mouse hematopoietic stem cell markers include cells that are lineage negative (e.g., T cell, B cell and myeloid markers), stem cell antigen-1 (Sca-1) positive and c-kit positive. These cells are LSK cells (i.e., lineage negative, Sca-1 positive and kit positive). LSK cells contain both long term repopulating and short term repopulating cells. Further markers may be used to identify long term repopulating hematopoietic stem cells. For example a CD34 marker can be used to identify LSK34neg cells as long term repopulating cells. CD135 and CD34 markers can be used to identify LSK135neg34neg cells as long term repopulating cells. CD48 and CD150 markers can be used to identify LSK48neg150posit cells as long term repopulating cells. CD48, CD41 and CD150 markers can be used to identify LSK48neg41neg150posit cells as long term repopulating cells. The above-described markers can also be used to identify LSK135neg34neg48neg150posit cells as the most quiescent long term repopulating cells. Subsequent to expansion, known differentiation factors can be contacted or administered as required by the investigator, researcher or treating physician. The methods of this disclosure can provide an increase of about 5%, or alternatively about 10%, or alternatively about 15%, or alternatively about 20%, or alternatively about 25%, or alternatively about 30%, increase in the number of HSCs obtained after treatment as compared to the number of HSC obtained use of an alternative expansion factor or no factor at all.

In one aspect a method is provided for increasing hematopoiesis in a subject in need thereof, comprising administering to the subject an effective amount of a compound of Formula I:

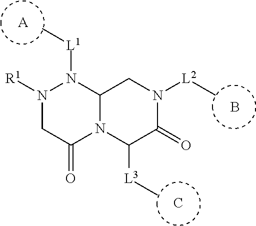

or a pharmaceutically acceptable salt thereof,
wherein
R¹ is hydrogen or C₁-C₆ alkyl;
ring A is cyclohexyl or phenyl, optionally substituted;
ring B is aryl or a nitrogen-containing heteroaryl, optionally substituted;
ring C is phenyl substituted with hydroxyl;
each L¹, L² and L³ is independently C₁-C₆ alkyl, —CONR²— or —CONR²—X—C₁-C₆ alkyl-, each optionally substituted; R² is hydrogen or C₁-C₆ alkyl; and
X is a bond or a 5-6 membered heterocycle containing up to 3 ring heteroatoms, and/or

a composition comprising, or alternatively consisting essentially of, or yet further consisting of one or more of the above compounds and a carrier, such as a pharmaceutically acceptable carrier.

In one embodiment of the method, the subject is a human or an animal, e.g., a mammal, a feline, canine, an equine, a bovine, an ovine, a murine or a rat. In one embodiment of the method, the subject is suffering from, or is susceptible to, decreased or depressed hematopoiesis or blood cell levels. In one embodiment of the method, the decreased or depressed hematopoiesis or blood cell levels are caused by chemotherapy, radiation therapy, bone marrow transplantation therapy or congenital anemia. In further embodiments of the method, the decreased or depressed hematopoiesis or blood cell levels are caused by an unintended exposure to radiation due an accidental release of radiation at, for example, a power plant. Thus, in one aspect, the methods are used to treat conditions caused by chemotherapy, radiation therapy or exposure, bone marrow transplantation or anemia, e.g., congenital anemia by administering to a subject in need thereof an effective amount of a compound of Formula I or a salt thereof, alone or in combination with another therapy as described.

In one embodiment, the method further comprises administering to the subject an effective amount of a CBP/β-, and/or a γ-catenin antagonist that promotes hematopoietic stem cell (HSC) differentiation, wherein the CBP/β-, and/or a γ-catenin antagonist is not a compound of Formula I or a pharmaceutically acceptable salt thereof, and/or a composition comprising one or more compounds as described herein. In one embodiment of the method, the CBP/β-, and/or a γ-catenin antagonist is PRI-724 or ICG-001.

In one embodiment, the method further comprises administering to the subject an effective amount of a supportive growth factor such as granulocyte-colony stimulating factor (G-CSF) and/or erythropoietin (EPO). The administration can be concurrent, prior to or subsequent to the administration of a compound of Formula I and/or a CBP/β-, and/or a γ-catenin antagonist, as determined by the treating physician or veterinarian.

In another aspect, a method is provided for enhancing expansion of a hematopoietic stem cell (HSC), comprising contacting the HSC with a compound, wherein the compound is of Formula I or a pharmaceutically acceptable salt thereof, and/or a composition comprising one or more compounds as described herein. In yet a further aspect, a method is provided for maintaining pluripotency of mouse and Human ES cells and Human iPS cells.

In one embodiment of the method, the cell is autologous or allogeneic and the contacting is in vitro or in vivo. When the contacting is done in vitro, the duration of the contacting step may last for about 8 hours, 12 hours, 18 hours, 24 hours, 30 hours, 36 hours, 42 hours, 48 hours, or a duration between any two of these values. When contacting in vivo, the administration amounts and dosages will be determined by the treating physician.

In one embodiment, the method further comprises contacting the HSC with a CBP/β-, and/or a γ-catenin antagonist that promotes hematopoietic stem cell (HSC) differentiation, wherein the CBP/β-catenin antagonist is not a compound of Formula I or a pharmaceutically acceptable salt thereof. In one embodiment of the method, the CBP/β-, and/or a γ-catenin antagonist is PRI-724 or ICG-001. When the contacting is done in vitro, the duration of the contacting step may last for about 8 hours, 12 hours, 18 hours, 24 hours, 30 hours, 36 hours, 42 hours, 48 hours, or a duration between any two of these values. When contacting in vivo, the administration amounts and dosages will be determined by the treating physician.

In another aspect, a method is provided for inhibiting an interaction between a β-, and/or a γ-catenin protein and a p-300 protein in a subject, the method comprising administering to the subject an effective amount of a compound of Formula I or a pharmaceutically acceptable salt thereof.

In one embodiment, the method further comprises administering to the subject a CBP/β, and/or a γ-catenin antagonist that promotes hematopoietic stem cell (HSC) differentiation, wherein the CBP/β-, and/or a γ-catenin antagonist inhibits the interaction between a β-, and/or γ-catenin and a CBP protein in the subject and is not a compound of Formula I or a pharmaceutically acceptable salt thereof. In one embodiment of the method, the CBP/β-, and/or a γ-catenin antagonist is PRI-724 or ICG-001.

### IV. EXAMPLES

Unless stated otherwise, the abbreviations used throughout the specification have the following meanings:
eq=equivalent
g=gram
kg=kilogram
L=liter
M=molar
mg=milligram
mL=milliliter
N=normal
nM=nanomolar
pM=picomolar
μM=micromolar
° C.=degree Celsius
DIEA=N,N-diisopropylethylamine
HATU=N-[(dimethylamino)-1H-1,2,3-triazolo-[4, 5-b]pyridin-1-ylmethylene]-N methyl methanaminium hexafluoro phosphate N-oxide
CDI=1,1′-carbonyldiimidazole
FACS=fluorescence-activated cell sorting
EC=enteric coated
IR=immediate release
G-CSF=granulocyte-colony stimulating factor
EPO=erythropoietin
HSC=hematopoietic stem cell
TLC=thin layer chromatography
CO-IP=co-immunoprecipitation
BMT=bone marrow transplantation
BrdU=5-bromo-2′-deoxyuridine
STF=stem cell factor

### Example 1: Representative Synthetic Procedure

Many of the compounds described herein were prepared according to modified versions of known synthetic procedures such as those described in U.S. Pat. Nos. 6,762,185; 7,531,320; 7,232,822; 7,563,825, and 7,598,253; and U.S. Patent Application Publication Nos. 20120296067; 20120088770; and 20110263607. The synthetic procedures from these patents and patent applications are incorporated herein by reference.

For example, a bromoacetal resin (Advanced ChemTech) was added to a solution of each amine (5 equiv, YH-249; 2-(aminomethyl)-6-chloro-N,N-dimethylaniline, YH-250; 2-(aminomethyl)-4,6-dichloro-N,N-dimethylaniline) and DIEA (5 equiv.) in DMSO. The reaction mixture was shaken at 60° C. for 36 hours. The resin was washed with DMF, MeOH, and DCM.

Fmoc-Tyr(tBu)-OH (4 equiv.), HATU (4 equiv.) and DIEA (12 equiv.) in DMF were added to the resin. After the reaction mixture was shaken for 8 hours at room temperature, the resin was washed with DMF, MeOH, and DCM. The resin was added to 25% piperidine in DMF and the reaction mixture was shaken for 1 hour at room temperature and washed with DMF, MeOH, and DCM.

2-(2-(((9H-Fluoren-9-yl)methoxy)carbonyl)-1-methylhydrazinyl)acetic acid (4 equiv.), HATU (4 equiv.), and DIEA (12 equiv.) in DMF were added to the resin. After the reaction mixture was shaken for 8 hours at room temperature, the resin was washed with DMF, MeOH, and then DCM.

The resin was added to 25% piperidine in DMF and the reaction mixture was shaken for 1 hour at room temperature and washed with DMF, MeOH and DCM.

CDI (4 equiv.) in DCM was added to the resin. The reaction mixture was shaken for 8 hours at room temperature. Soon after the resin was washed with DCM, (2,3-dimethoxyphenyl) methanamine (8 equiv.) in DCM was quickly added to the resin. The reaction mixture was shaken for 8 hours at room temperature. The resin was washed with DMF, MeOH, and DCM.

The resin was treated with formic acid for 2 days at room temperature. After the resin was removed by filtration, the filtrate was condensed under a reduced pressure. The crude product was purified by preparative TLC (ANALTECH, thinlayer chromatography plate, AcOEt as a developing solvent) to yield the product.

### Example 2: Screening for Compounds that Block the Interaction Between β-Catenin and p300

The binding between β-catenin (or γ-catenin,) with CBP or with p300 plays a pivotal role in the determination of stem/progenitor cell fate: binding of β-, and/or a γ-catenin with CBP promotes expansion (i.e., clonal proliferation), while binding of β-, and/or a γ-catenin with p300 promotes cell differentiation.

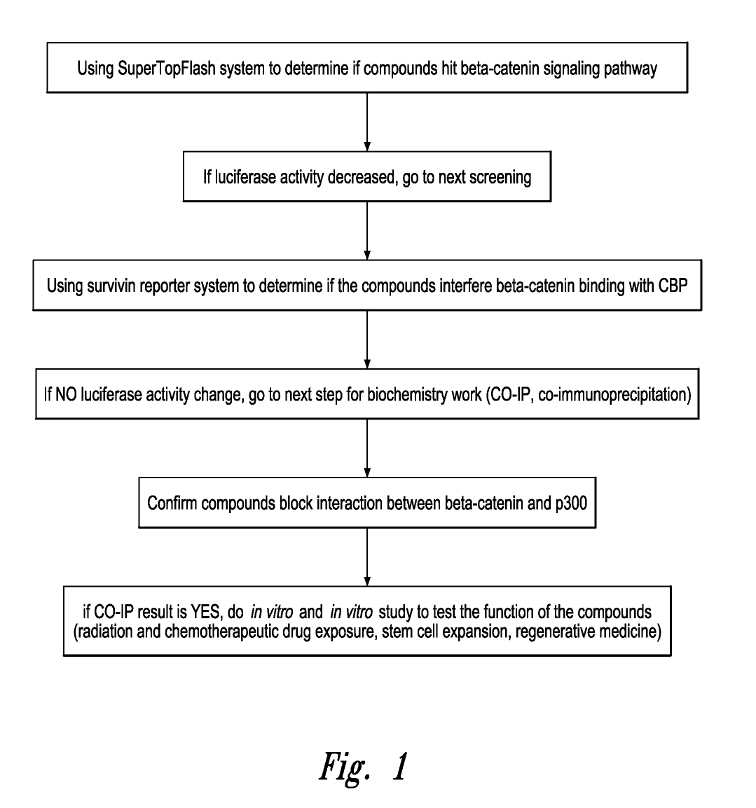

Compound ICG-001, by binding to the N-terminus of CBP, blocks the interaction between β-catenin and CBP and thereby increases the binding of β-catenin with p300. Thus, ICG-001 promotes cell differentiation. Also, ICG-001 has been described to significantly inhibit survivin gene expression. To discover compounds that can block the interaction between β-catenin with p300, a two-step screening system was developed. This screening system helped identify compounds that inhibit Wnt/β-catenin pathway but have no significant effect on survivin expression. See the flowchart in FIG. 1.

A. Wnt/β-Catenin Activity: The Initial SuperTOPFLASH Screen.

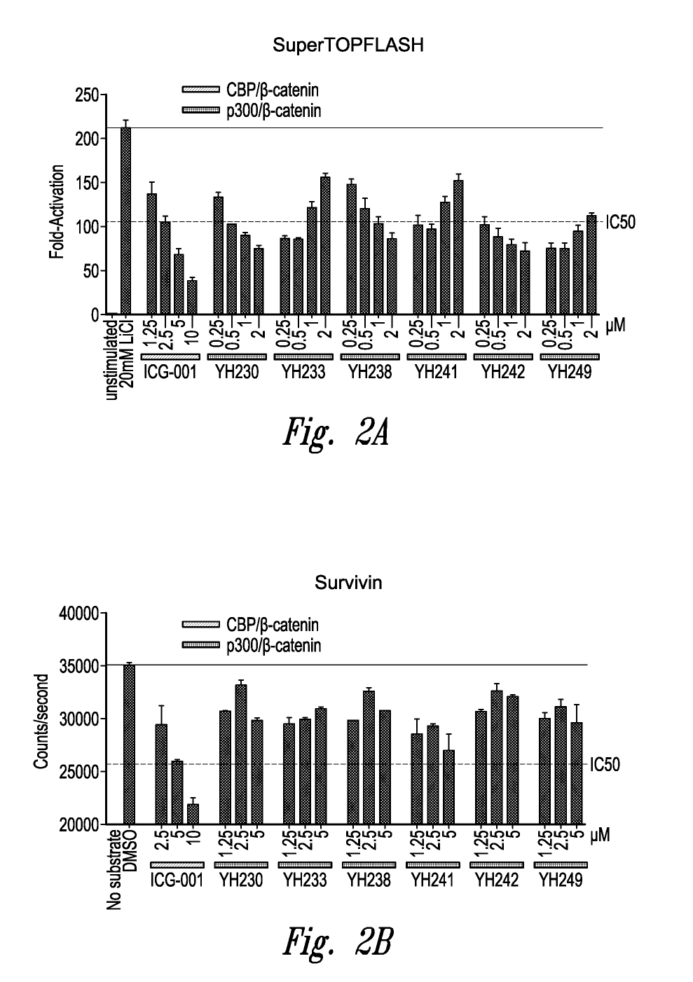

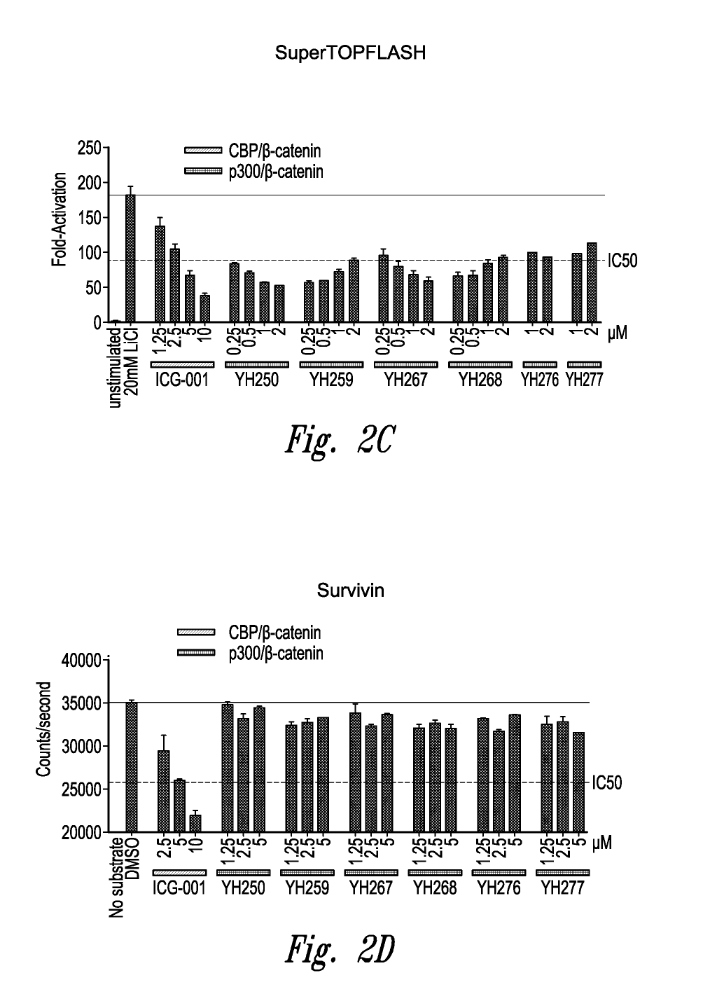

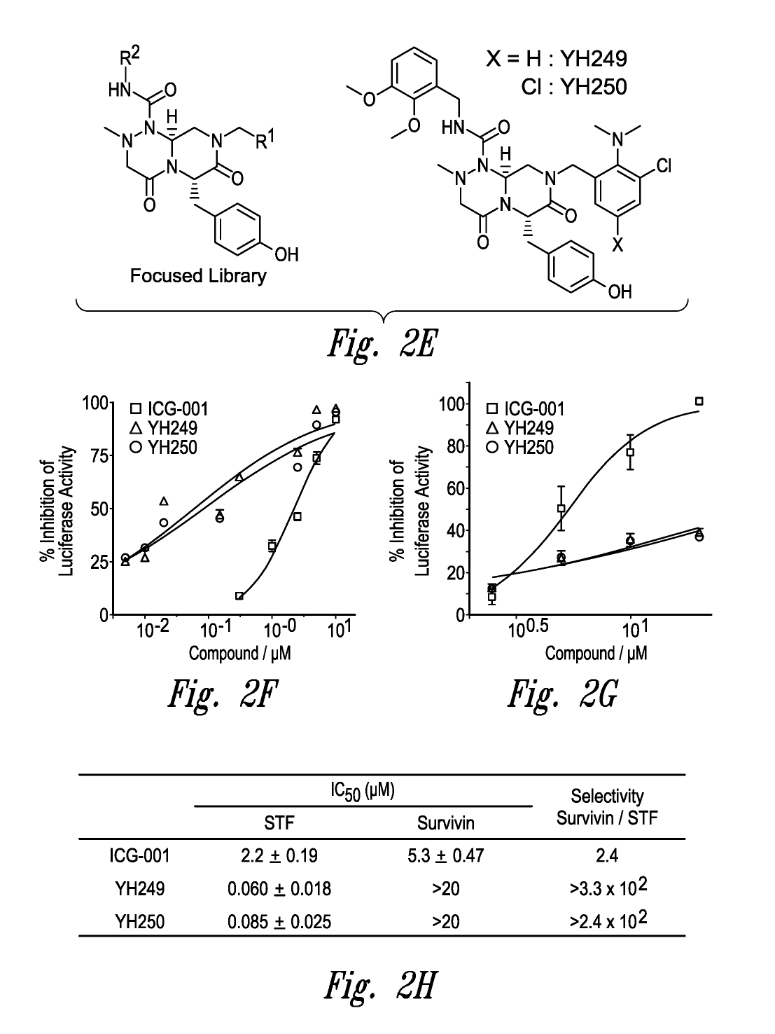

Hek293 cells were stably transfected with plasmid that contains multiple TCF/LEF fragments in front of Luciferase gene. Wnt/β-catenin activity was determined from luciferase expression levels. For example, when ICG-001 was added in this system, luciferase activity was decreased, indicating ICG-001 inhibits Wnt/β-catenin pathway. See FIGS. 2A and 2C. Briefly, stably transfected Hek-293, STFI.1 (Wnt/β-catenin-driven luciferase) and Survivin-Hek-293 (1 Kb Human survivin promoter-driven luciferase) cells were maintained in DMEM, 10% FBS, Pen/Strep with 400 μg/mL G418 or 2 μg/mL puromycin, respectively. To check the activity of ICG-001, YH249, and YH250, the cells were split into white-opaque 96-well plates at 10,000 cells per well and allowed to attach overnight. For SuperTOPLASH, the inhibitors were added to the cells and pre-incubated for 1 hour prior to stimulation with 10 mM Li Cl for 24 hours. Luciferase activity was read using BrightGlo (Promnega) luciferase substrate. For survivin/luc, the inhibitors were added to the cells and incubated for 24 hours prior to readout. To calculate IC₅o values, the resulting curve was fitted to a four-parameter logistic model (4PL) using GraphPad Prism 5 software.

B. Survivin Expression Level: The Secondary Screen.

An endogenous human survivin 1 kb promoter was inserted before a luciferase gene. For example, when ICG-001, a known agent that promotes hematopoietic stem cell (HSC) differentiation, was added to this survivin-reporter system, survivin expression level was significantly decreased in a dose dependent manner. See FIGS. 2B and 2D. From this screen, two close structural analogs, YH249 and YH250 (FIG. 2E) that were potent inhibitors of the SuperTopFlash reporter but demonstrated little or no inhibition of the survivin/luciferase reporter in the same cell line, were selected for further characterization and target validation. YH249 and YH250 (FIG. 2E) both had IC50 values below 100 nM in the SuperTopFlash assay (FIG. 2F), yet did not inhibit the survivin/luciferase reporter up to 20 μM (FIG. 2G). These compounds therefore exhibit >200 fold selectivity for the p300/p-catenin interaction relative to the homologous CBP/p-catenin interaction (FIG. 2H). R1 and R2 were diversified in this study. Percent inhibition of luciferase activity was calculated according to the following equations; % inhibition of STF 1.1 reporter activity=(I−RLU_{(10 mM LiCl) treated/RLU(10 omM LiCl) vehicle}×_{control})100, % inhibition of Survivin reporter activity=(1−RLUtreated/RLU vehicle control)×100. Error bars indicate the mean±SE of 3 separate experiments. Selectivity (FIG. 2H) was calculated by IC₅₀ value for Survivin-Hek-293/IC₅₀ value for STF 1.1.

### Example 3: “Pull-Down” Assays: The Compounds Interfered with Interactions Between β-catenin and p300

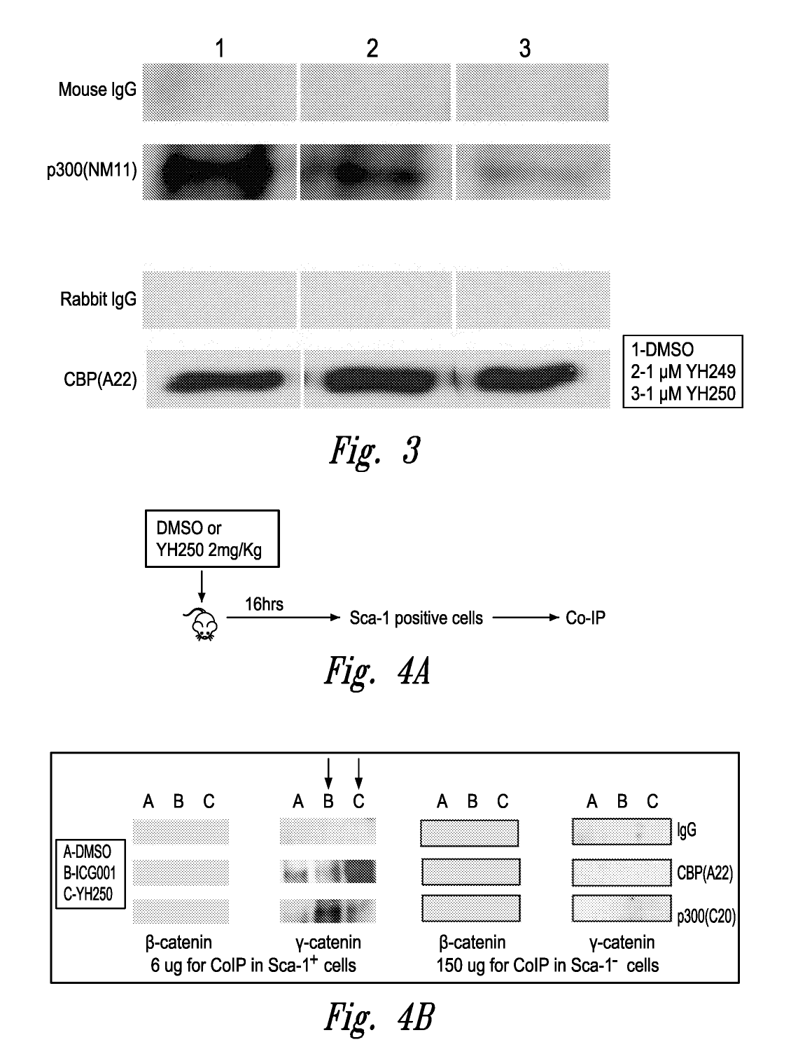

YH249 and YH250 were selected from the compounds described herein for further testing. Co-immunoprecipitation (CO-IP) was performed with mouse myoblast C2Cl2 cells. Cells were treated with DMSO (control), or YH249, or YH250 for 24 hours. Cell lyses were pulled down with anti-p300 or anti-CBP antibodies, run on gel and probed with anti-β-catenin antibody. As shown in FIG. 3, in the presence of YH250, there were significantly decreased levels of β-catenin in the p300 pull down lane, indicating YH250 interfered with the interaction between β-catenin and p300. To demonstrate that YH249 bound directly to p300, an affinity column strategy was used (Enamni, K. H., et al., Proc. Natl. Acad. Sci. 101, 12682-12687, 2004). A biotinylated version of YH249 (Bio-249) was prepared via the propargyl amide variant. Using a biotin with a linker and a terminal azide residue, we were able to couple the biotin to the YH249 core via a 3+2 cyclization reaction to provide Bio-249. Bio-249 was incubated with lysates from C2Cl2 myoblasts, either in the presence or absence of competitor YH249 for 4 h at 4° C. and then bound to streptavidin agarose beads. After extensive washing the bound proteins were eluted and run out on an agarose gel. Bio-249 very cleanly precipitated a high molecular weight band that was not present in the biotin control and was strongly competed out by free YH1249. Immunoblotting with a p300 specific antibody confirmed the identity of the band, whereas immunoblotting with a CBP specific antibody confirmed a lack of binding and specificity for p300. Taken in total, these data demonstrated that YH249, by directly binding to p300 represents a potent, specific, direct antagonist of the p3 00113-catenin interaction

### Example 4: “Pull-Down” Assay: The Compounds Interfered with Interaction Between γ-Catenin and p300 in Bone Marrow Hematopoietic Stem Cell Population

YH250 was used in this study. Female C₅₇/Black mice were administrated with either DMSO (solvent control) or YH250 (2 mg/kg) subcutaneously. 24 hrs later, bone marrow Sca-1posit cells were isolated with Anti-Sca-1 microbeads (Miltenyi Biotec, Cat#130-092-529) for co-immunoprecipitation (FIG. 4A). Cells were then lysed and incubated with either anti-CBP (A22) or anti-p300 (C20) antibodies. Then, the immunoprecipitation complex were electrophoresed and detected with antibodies to either β-catenin or γ-catenin. As shown in FIG. 4B, there is no detectable level of β-catenin in Sca-1posit and Sca-1negt cells. In Sca-1posit cells, YH250 inhibits p300/γ-catenin interaction and promotes CBP/γ-catenin interaction (lane C). ICG-001 (CBP/catenin antagonist) is opposite to YH250, in that ICG-001 inhibits CBP/γ-catenin interaction and promotes p300/γ-catenin interaction (lane B). This confirms that YH250 directly disrupts the p300/catenin interaction thereby enhancing the CBP/catenin interaction in HSPCs.

### Example 5: Gene Expression Analysis for Axin2 and BirC 5 (Survivin) in YH250 Treated Animals

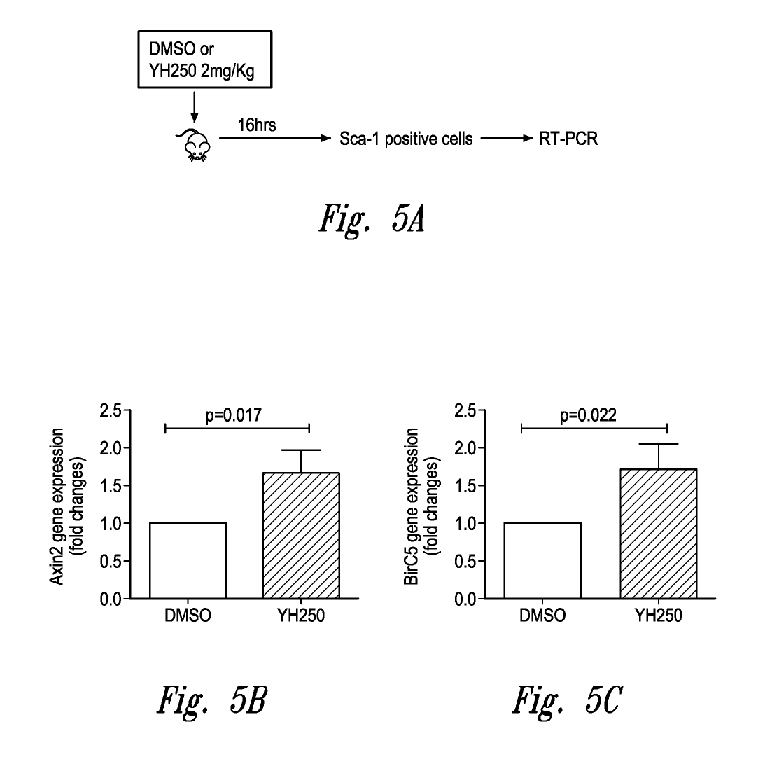

Axin2 is a classical Wnt/catenin target genes and plays important regulatory roles in Wnt signaling. The inhibitor of apoptosis (IAP) family member survivin (Birc5) is another Wnt/catenin target gene. We have previously demonstrated that ICG-001, by antagonizing the CBP/catenin interaction, down-regulates survivin expression in a variety of cell types both in vitro and in vivo. To study YH250 effects in bone marrow stem cell population gene expression, female C57/Black mice were treated with DMSO or YH250 (2 mg/kg) overnight and bone marrow Sca-1posit cells were isolated. Then, total RNA were extracted for cDNA synthesis and qPCR assay (FIG. 5A). As shown in FIGS. 5B-5C, both Axin2 and survivin gene expression were up regulated by YH250 in the Sca-1⁺ population of the treated animals.

### Example 6: RNA Sequencing of Long-Term Repopulating HSCs (LTR-HSC)

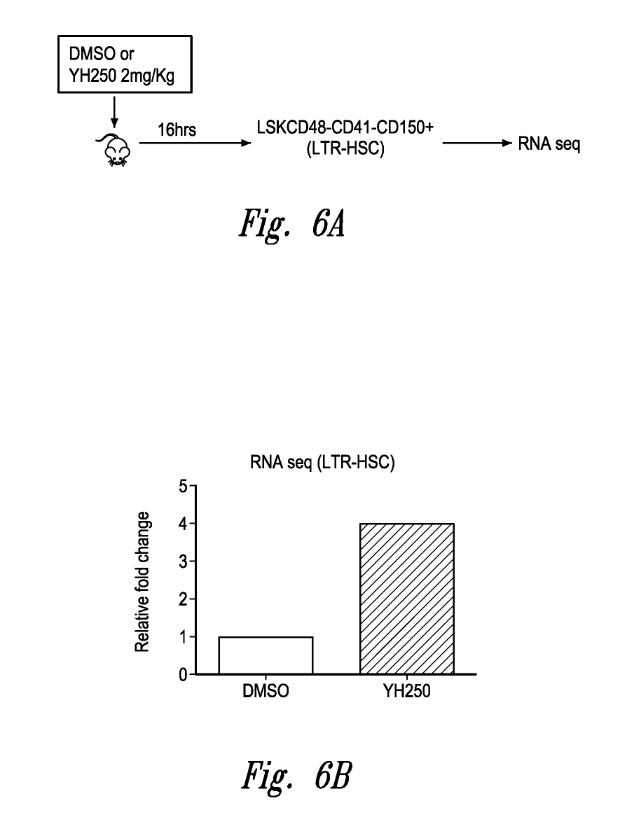

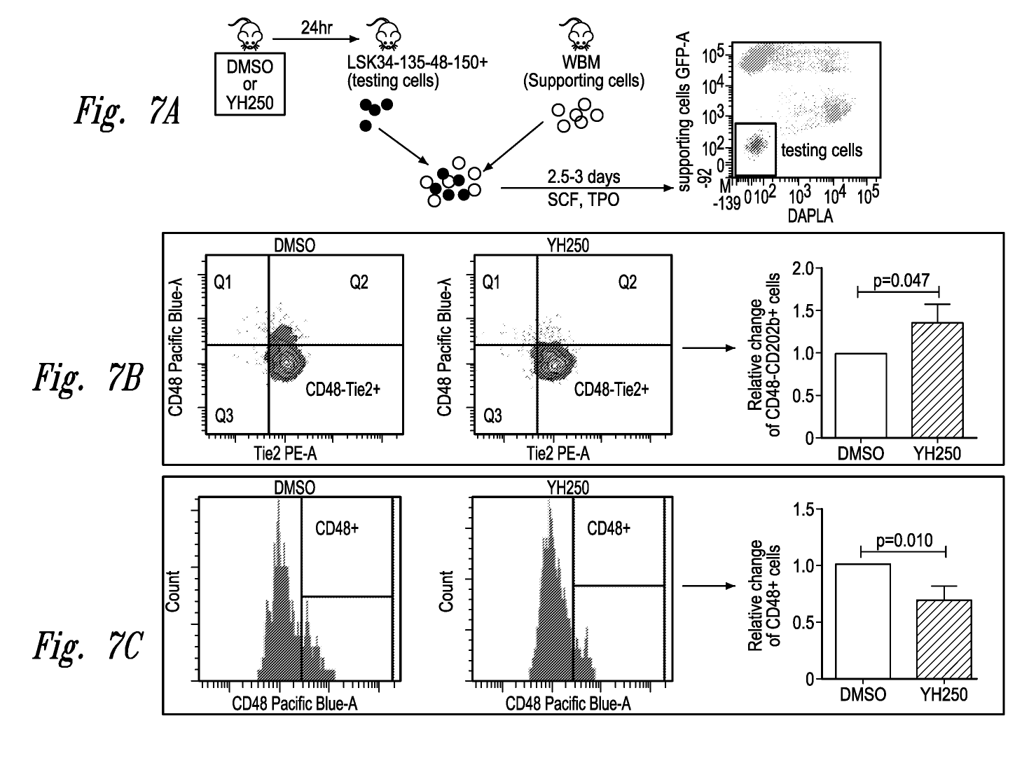

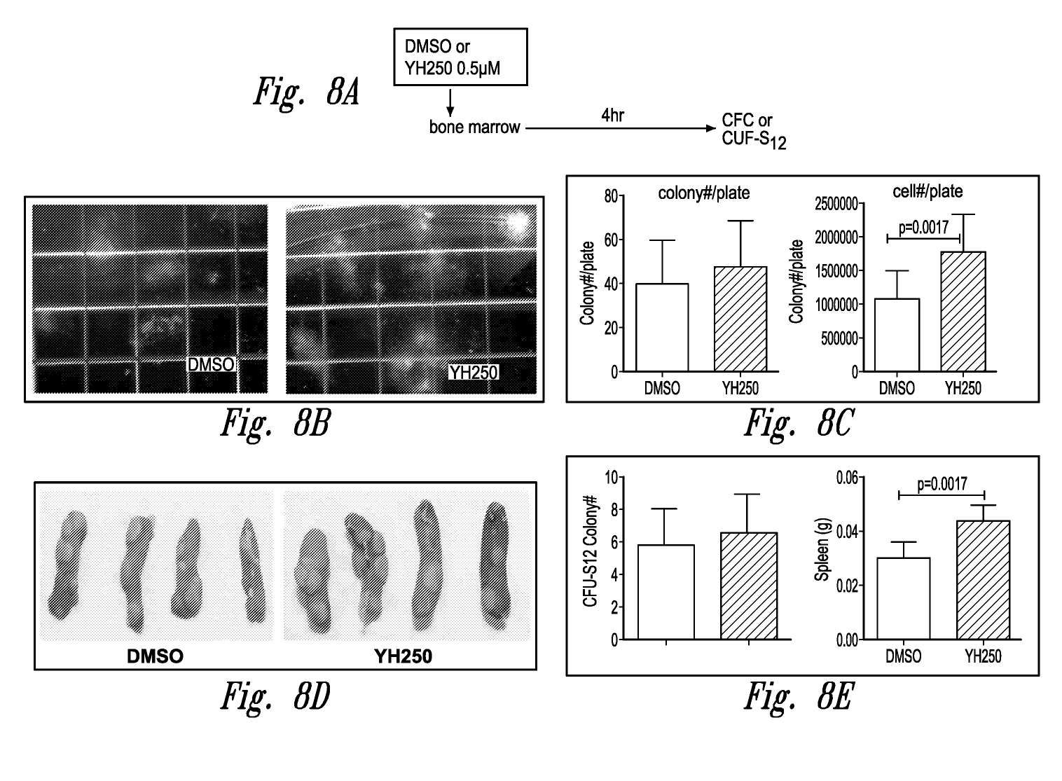

DMSO or YH250 (2 mg/kg) were administrated to female C57/Black mice. 16 hours later, long-term repopulating HSC (Lin-negt/Sca-1-posit/c-kit-posit/CD41negt/CD48negt/CD150posit cells were purified with fluorescent flow cytometry sorting (FACS) for RNA isolation and RNA sequencing (FIG. 6A). As shown in FIG. 6B, ID2 (Inhibitor of DNA binding protein 2) gene expression was up-regulated about 4-fold in YH250 treated animals. ID2 has been considered as a negative regulator of cell differentiation and plays important roles in HSC expansion.

Other genes that were significantly upregulated in the LTR-HSC and not in the progenitor population included CD52 (3.09 fold), a membrane protein that is found on the surface of HSPCs, and the tetraspanin protein CD53 (2.79 fold), which is associated with the CD34⁺CD133⁺ cord blood HSC population. Based upon these results, we investigated the ability of YH250 to promote HSC non-differentiative proliferative divisions in vitro and in vivo.

Table 2 below shows RNA sequence analysis of LSK34135-48CD150⁺ cells from YH250 vs DMSO treated animals:

| | | | | Fold-Change | Fold-Change |
|---|---|---|---|---|---|
| Chromosome | strand | gene_type | gene_symbol | (LSK150⁺41⁻48⁻) | (LSK150⁺41⁺48⁺) |
| 4 | − | protein | Cd52 | 3.09 | |
| 7 | + | protein | Cd79a | 5.49 | |
| 11 | − | protein | Tmed4 | 2.06 | |
| 12 | − | protein | Id2 | 3.97 | |
| 15 | − | protein | Ly6c2 | 7.17 | |
| 17 | + | protein | Ndufaf7 | 3.45 | |
| 18 | + | protein | Cd74 | 2.30 | |
| 1 | + | protein | Mrpl30 | 2.07 | |
| 3 | − | protein | Car1 | 46.29 | −12.1 |
| 3 | + | protein | Casp6 | 2.56 | |
| 6 | − | protein | Cd69 | 2.44 | |
| X | − | protein | Il2rg | 2.03 | |
| 9 | + | protein | Ldlr | −3.57 | |
| 9 | − | protein | Pias1 | 2.02 | |
| 2 | − | protein | Psmf1 | 2.48 | |
| X | + | protein | Mid1 | −3.98 | −2.8 |
| 2 | − | protein | Cdc123 | 2.13 | |
| 10 | − | protein | Dtx3 | −2.12 | |
| 3 | − | protein | Cd53 | 2.79 | |
| 4 | + | protein | A430005L14Rik | 2.07 | |
| 5 | − | protein | Igj | 3.36 | |
| 9 | − | miRNA | Gm26377 | −58.02 | −3.19 |
| 6 | + | IG_C_gene | Igkc | 3.93 | |
| 12 | − | IG_C_gene | Ighm | 6.14 | 2.36 |
| 16 | − | protein | Iglc2 | 4.58 | |
| 11 | − | protein | Tgtp1 | 2.85 | |
| 6 | + | IG_LV_gene | Igkv14-126 | 8.00 | |
| 16 | + | protein | Gm21897 | −2.18 | |
| 12 | − | IG_LV_gene | Ighv11-2 | 8.18 | 4.42 |
| 6 | − | IG_LV_gene | Igkv19-93 | 21.16 | 3.12 |
| 7 | − | antisense | Kcnq1ot1 | −2.37 | |
| X | + | lincRNA | RP23-99K18.3 | −2.28 | |

### Example 7: HSC Differentiation Assays

DMSO or YH250 (2 mg/kg) were administrated to female C57/BL mice. (Mice were purchased from JAX stock#000664 for CD45.2 or Stock#002014 for CD45.1). 24 hours later, long-term repopulating HSC (LTR-HSC, Lin-negt/Sca-1-posit/c-kit-posit/CD34negt/CD135negt/CD48negt/CD150posit cells were purified with fluorescent flow cytometry sorting (FACS). Then, the purified cells were co-cultured with whole bone marrow cells isolated from GFP-transgenic mice (C57BL/6Tg-UBC-GFP, JAX stock#004353), which serves as supporting system in stem cell medium (Stemspan, Stemcell Technologies, Cat#9650) supplemented with SCF (stem cell factor 10 ng/ml) and TPO (thrombopoietin, 10 ng/ml). 2.5-3 days after co-culture, cells were harvested and analyzed for surface markers of CD48 and CD202b (Tie-2) (FIG. 7A). It has been demonstrated that cells maintaining CD48negt/CD202bposit phenotype retain long-term repopulating HSC property while cells acquired CD48 expression are differentiated and lost long-term repopulating HSC activity. As depicted in FIG. 7B, there are significantly more non-differentiated CD48negt/CD202bposit cells from YH250 treated cells compared with DMSO treated controls (p=0.04). There are significantly less CD48posit cells in YH250 treated cells (FIG. 7C, p=0.010).

### Example 8: Colony-Forming Cell (CFC) and CFU-S12 Assay

Brief (less than 24 hours) treatment of HSC with YH250 or DMSO was conducted before the CFC or CFU-S₁₂ assay. See the experimental design shown in FIG. 8A. Mouse bone marrow cells were treated with DMSO (solvent control) or YH250 for 4 hours in serum free HSC expansion medium before the cells were recovered for a colony forming assay. 7-8 Days after cell plating in methylcellulose medium (Stemcell Technologies, Methocult GF M3434, Cat#3434), colonies were scored, and the cells from each plate were recovered and counted. As shown in FIGS. 8B and 8C, the colony number were not significantly different, but the cells with YH250 treatment had increased cell proliferation activity and resulted in increased cell number in each plate.

In CFU-S12 assay, cells treated with DMSO or YH250 were recovered from plate and injected to lethally irradiated mice (FIG. 8A), 12 days later, animals were euthanized to check spleen colony formation and spleen weight. As shown in FIGS. 8D and 8E, YH250 treated cells give larger colonies on spleen and that resulted in heavier spleen.

### Example 9: Brief Incubation of Bone Marrow Cells with p300/Catenin Antagonist YH250 Resulted in In Vitro Expansion of Hematopoietic Stem Progenitor Cells (HSPC)

Isolated bone marrow nucleus cell or HSC from bone marrow or peripheral blood (after HSC mobilization procedure as commonly practiced in the clinic) were cultured in medium containing p300/β-catenin antagonist (such as YH250 or YH249) at proper concentration. The medium can be commercially available serum free HSC culture medium, such as (but not limited) QBSF-60 (Quality Bio), StemPro-34 SFM (Gibco), StemSpan (Stemcell technologies). The medium may contain one or more hematopoietic stem/progenitor cell growth factor(s), such as, but not limited to stem cell factor (SCF), Thrompoietin (TPO), Flk3 ligand, IL-6, GCSF and IL-3. The medium will be totally or partially replaced periodically during cell culture. Cultured cells can be analyzed for HSC phenotype with FACS. Cells can be harvested at proper time for clinical application.

It is contemplated that a relatively brief incubation of human bone marrow (or HSC) with the p300/-catenin antagonists described herein, such as YH250 or YH249, will enhance cell proliferation. It is further contemplated that patients will have quicker and better hematopoiesis in bone marrow/stem cell transplantation by using such treated bone marrow or HSC.

Thus, an exemplary protocol comprises isolating whole bone marrow or HSC from bone marrow or peripheral blood (after HSC mobilization procedure as commonly practiced in the clinic). Red blood cells are removed using conventional methods, such as lysis buffer or centrifugation. To the centrifuged cells, add medium with YH250 or YH249 at 1 pM to 1 mM. The medium can be commercially available serum free HSC culture medium, such as (but not limited) QBSF-60 (Quality Bio), StemPro-34 SFM (Gibco), StemSpan (Stemcell technologies). The cells cultured under conventional methods known in the art and then are harvested after short time (less than 24 hours) incubation, the compounds are removed (by centrifugation or other methods) and used for bone marrow transplantation or other use.

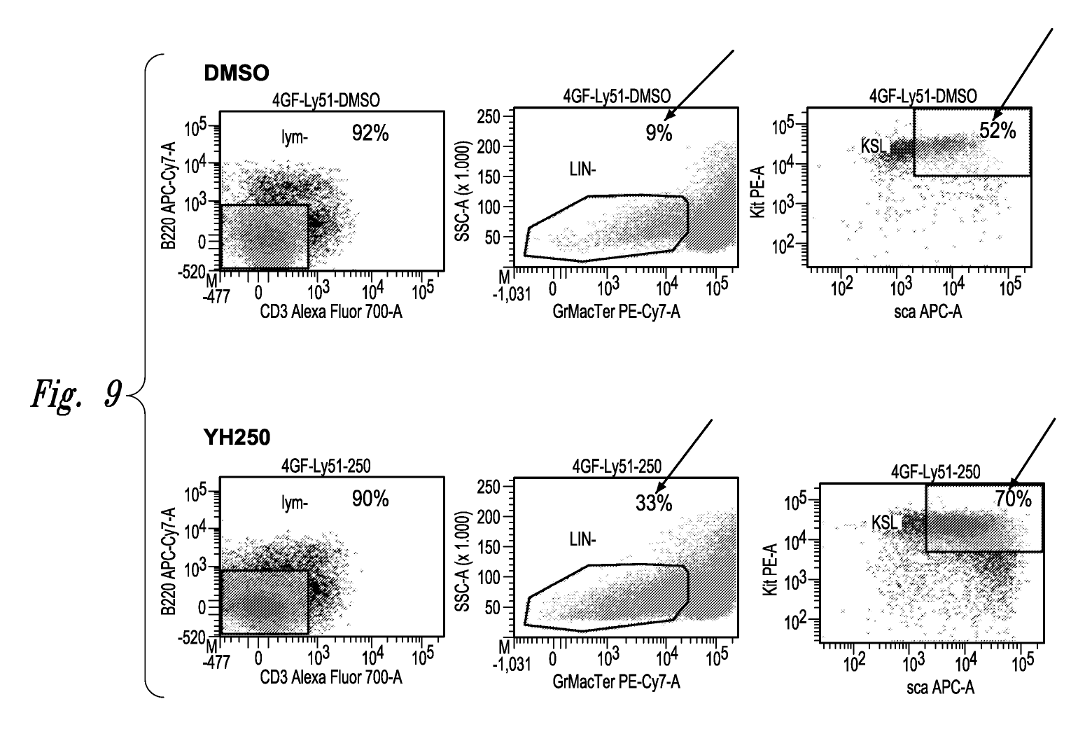

Bone marrow cells were treated with DMSO or YH250 (0.5 μM) for 4 hours, then the compound was removed and the cells cultured in QBSF-58 medium for 14 days with 10 ng/ml of SCF, TPO, Flt3 ligand, and IL-6. At the end of culture, cells were recovered and analyzed for LSK cells with FACS. As shown in FIG. 9, the percentage of Lineage negative and LSK cells were significantly higher in YH250 treated samples compared with DMSO treated samples.

### Example 10: Extended Incubation of Bone Marrow Cells with p300/-Catenin Antagonist Resulted in In Vitro Expansion of HSPC

Mouse bone marrow cells were isolated and cultured in QBSF-58 medium with DMSO or YH250 0.5 μM (medium contains 10 ng/ml of SCF, TPO, Flt3 ligand, and IL-6) for 3 days and cells are recovered and analyzed for HSC surface markers with FACS.

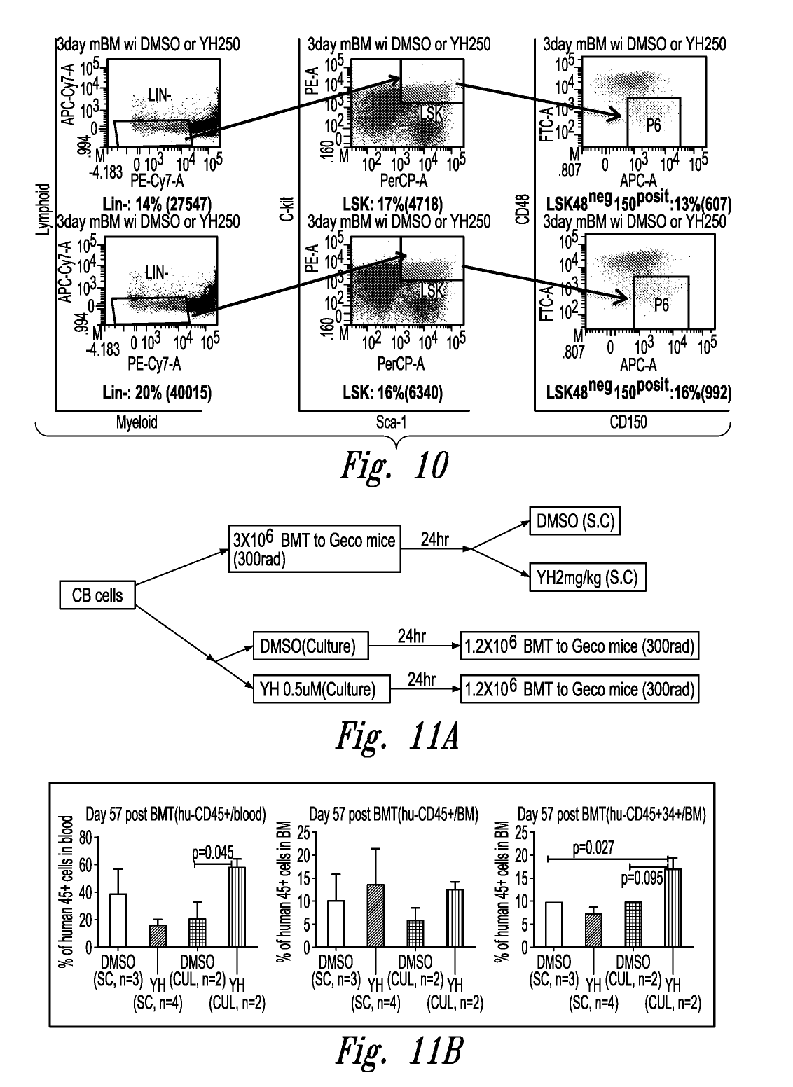

FIG. 10 depicts the results of FACS analysis. YH250 containing medium gave more HSC (LSK48-150+, bottom panel) compared with DMSO containing medium (992 vs 607 cells for YH250 vs DMSO).

TABLE 3
The data from a 3-day in vitro culture of bone marrow cells (200,000 live cells were analyzed):

| | | | | | LSK | | | | | | | |
|---|---|---|---|---|---|---|---|---|---|---|---|---|
| | Lin-neg | | LSK | | 135 − 34− | | 135 − 34+ | | 135 + 34+ | | 48 − 150+ | |
| | % | Cell# | % | Cell# | % | Cell# | % | Cell# | % | Cell# | % | Cell# |
| DMSO | 14 | 27547 | 17 | 4718 | 10 | 456 | 19 | 885 | 69 | 3247 | 13 | 607 |
| YH250 | 20 | 40015 | 16 | 6340 | 7 | 445 | 25 | 1602 | 67 | 4263 | 16 | 992 |

### Example 11: Incubation of Cord Blood Cells with p300/-Catenin Antagonist Resulted in In Vitro Expansion of HSPC

Cord blood cells are enriched for HSPC. It has been considered as a source of hematopoietic and mesenchyme stem cell for stem cell therapy. However, the limited number of stem cells in one dose of cord blood cell restricted its clinical efficacy in stem cell transplantation. Since p300/-catenin antagonists have the potential to stimulate stem cell proliferation and expand stem cell pool, we used YH250 in cord blood cell study.

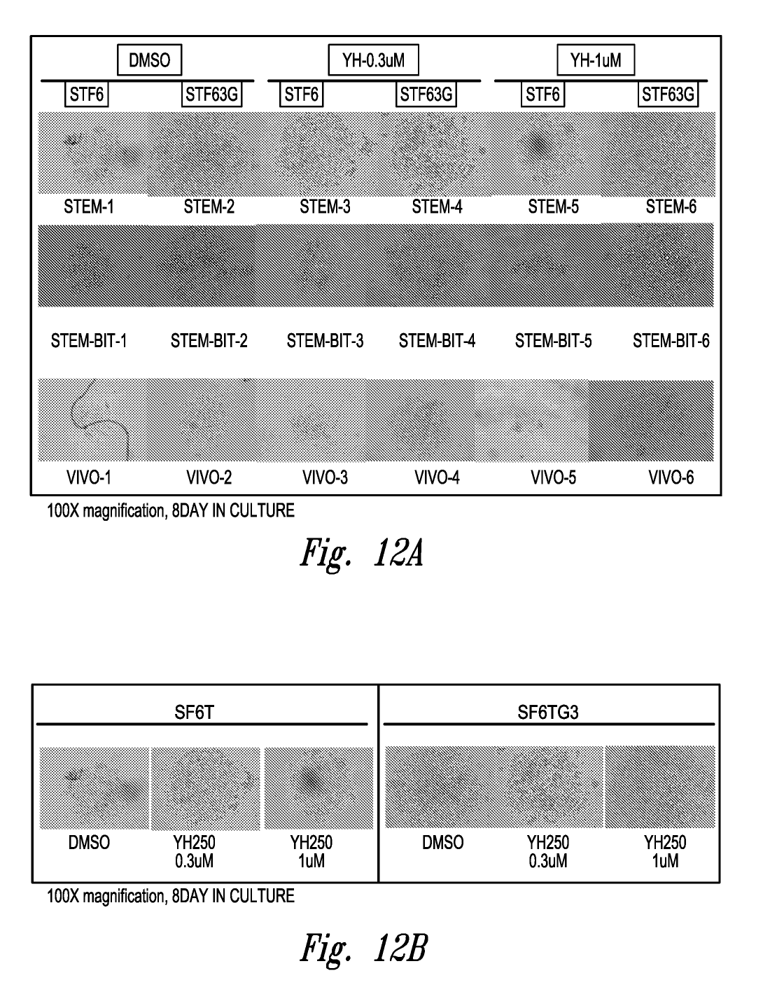

FIG. 11A depicts the experimental procedure. Cord blood cells were tested for repopulating ability in NSG mice (NOD-SCID IL-2g^{-/-}, JAX stock#005557. One method is to administrate p300/-catenin antagonist such as YH250 to recipient animals after BMT, another method is to incubate cord blood cells with YH250 in vitro for 24 hours before BMT. At day 57 post BMT, mice blood and bone marrow cells were analyzed for donor cell engraftment. As shown in FIG. 11B, YH250 pre-incubation gave the best engraftment in blood. And, compared with DMSO treated cells, YH250 in vitro treated cells give more human CD34+ cells in bone marrow.

### Example 12: Incubation of Cord Blood CD34+ Cells with p300/-Catenin Antagonist in the Presence of Different Growth Factor Combinations

It is further contemplated that longer treatments of HSC with a p300/-catenin antagonist at a proper concentration and time could expand cord blood HSC in vitro. The expanded cord blood cell HSC can be used in clinical for bone marrow transplantation (BMT). Using CD34+ cord blood cells (enriched with hematopoietic stem cells), different combination of growth factors in the presence or absence of p300/-catenin antagonist were used to expand hematopoietic stem cells in different serum free stem cell expansion mediums.

Cord blood CD34+ cells were purchased from Lonza, product code 2C-101B, Lot number 2F3441 and 2F3380. Cord blood cells for BMT were purchased from Allcells (Cat#003F, ID# CB 0809250).

Serum free stem cell mediums were purchased from Stemcell technologies (Stemspan II Cat#09605, and BIT 9500 Serum Substitute, Cat#9500) or Lonza (X-VIVO 20 with Gentamicin L-Gln, Phenol Red, Cat#04-448Q). Growth factors were purchased from R&D systems: recombinant human SCF (Cat#255-SC-010), recombinant human Flt2 ligand (Cat#308-FK-025), recombinant human thombopoietin (Cat#288-TP-005), recombinant human IL-6 (Cat#206-IL-010), recombinant human GCSF (Cat#214-CS-005/CF), recombinant IL-3 (Cat#203-IL-010).

Antibodies were purchased from eBioscience, anti-human CD34 (clone 563, Cat#561290), anti-human CD45 (clone 2D1, Cat#11-9459-42), anti-human CD38 (clone HB7, Cat#25-0388-42), anti-human CD45R (clone H100, Cat#45-0458-73), anti-human CD33 (clone WM-53, Cat#56-0338-42).

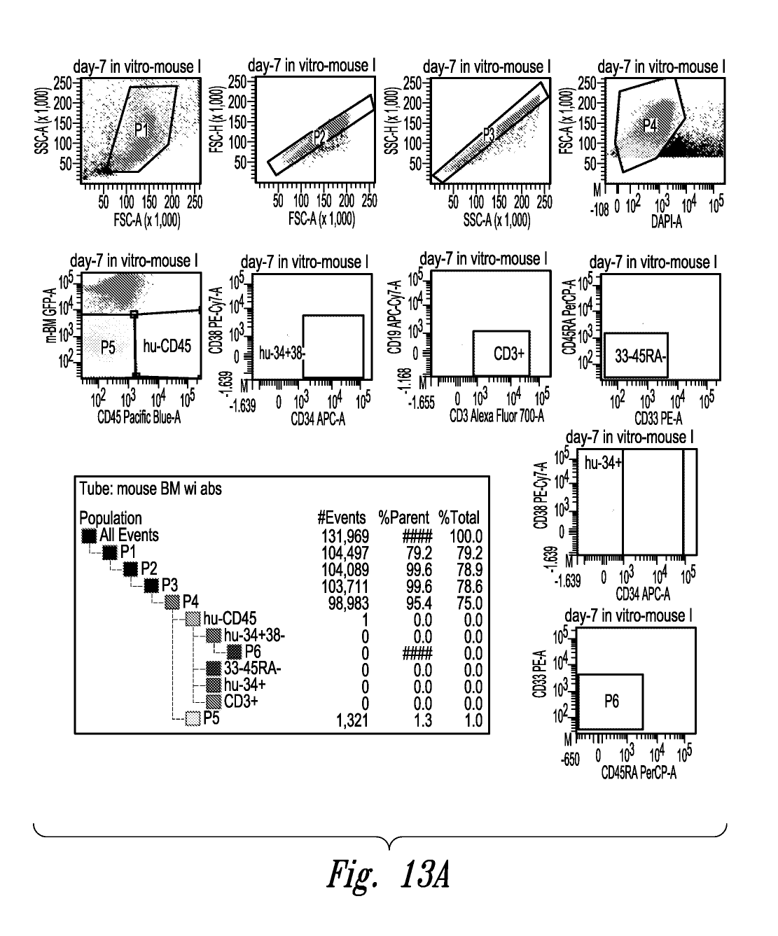

Cord blood CD34⁺ cells were cultured in serum free medium with different growth factor combination and different concentrations of YH250. We tested three serum free mediums with either 4 growth factors (4GF, or SF6T: SCF 100 ng/ml; Flt3 100 ng/ml, IL-6 20 ng/ml, and TPO 20 ng/ml) or 6 growth factors (6GF, or SF6TG3: SCF 100 ng/ml; Flt3 100 ng/ml, IL-6 20 ng/ml, TPO 20 ng/ml, GCSF 10 ng/ml, and IL-3 10 ng/ml). YH250 was at 0.3 μM or 1 μM concentration, DMSO, the solvent for YH250, was used as control. Frozen cord blood CD34+ cells were plated into 96 wells with different medium and different growth factors and different compound concentrations. At day 8 in culture, pictures were taken to record the cell growth and colony morphology (FIG. 12A). As shown in FIG. 12B, medium with 4GF at 1 M of YH250 gave colonies more close to “stem cell colony” morphology: cell size are small and unified, the colony is more compact.

### Example 13: Methods for Analyzing In Vitro Expanded Cord Blood CD34+ Cells

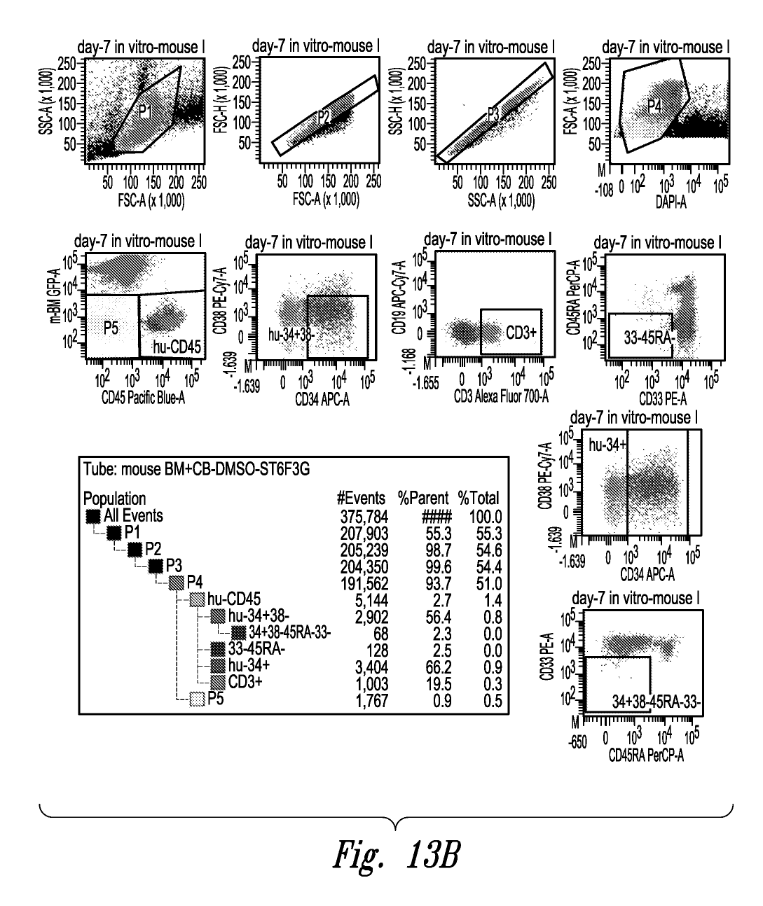

A method to analyze expanded cord blood CD34+ cell phenotype using FACS is established. In this study, cord blood cells were cultured in vitro with 4GF and different concentrations of p300/-catenin antagonist such as YH250. Because the limited CD34+ cells in culture system, GFP transgenic mouse bone marrow cells were isolated and added to the cells as carrier cells in FACS analysis. The bright GFP positive carrier cells were gated out and the targeting cord blood cells were further analyzed for their surface marker. FIG. 13A depicts carrier cell only in FACS analysis. GFP positive carrier cells do not give signal with anti-human-CD45 staining, thus there is no noise from carrier cells in this assay system. FIG. 13B shows cord blood CD45+ cell derived cells in culture system were gated for further analyzed for hu-CD45+ CD34+ and hu-CD45+CD34+CD38-CD45RA-CD33-(primitive HSC) cells.

### Example 14: Incubation of Cord Blood CD34+ Cells with p300/Catenin Antagonist Expanded Cord Blood HSC In Vitro

Cord blood CD34+ cells were cultured in medium containing either 4GF or 6GF with different concentration of p300/-catenin antagonist YH250. At 7^{th} day in culture, cells were recovered and mixed with carrier cells from GFP mice bone marrow. Antibodies to human HSC surface protein are added to the cells and human CD45posit cells were gated for FACS analysis. As shown in Table 4, in 4GF medium and 10 μM of YH250, there are more human CD45+CD34+ cells compared with DMSO (89.1% vs 79.1%). In 6GF medium, 10 μM of YH250 also gives more human CD45+CD34+ cells compared with DMSO (73.3% vs 66.2%, Table 5).

TABLE 4
Cell surface marker analysis in
medium containing 4GF and YH250

| SF6T in | | | | | |
|---|---|---|---|---|---|
| stem-pro | | | %34 + 38- | | |
| medium | %34+ | %34 + 38- | 45RA-33- | %33-45RA- | % CD3+ |
| DMSO | 79.1 | 64.5 | 3.9 | 4.5 | 12.2 |
| YH250-1 μM | 64.1 | 51.7 | 3.5 | 5.0 | 16.0 |
| YH250-3 μM | 84 | 65.5 | 5.2 | 5.8 | 9.5 |
| YH250- | 89.1 | 65.3 | 4.5 | 4.9 | 4.5 |
| 10 μM | | | | | |

TABLE 5
Cell surface marker analysis in
medium containing 6GF and YH250

| SF6T3G in | | | | | |
|---|---|---|---|---|---|
| stem-pro | | | %34 + 38- | | |
| medium | %34+ | %34 + 38- | 45RA-33- | %33-45RA- | % CD3+ |
| DMSO | 66.2 | 56.4 | 2.3 | 2.5 | 22.6 |
| YH250-1 μM | 62 | 53.9 | 4.4 | 5.8 | 20.1 |
| YH250-3 μM | 66.1 | 55.8 | 4.2 | 5.6 | 18.8 |
| YH250- | 73.3 | 57.9 | 4.2 | 6.1 | 13.8 |
| 10 μM | | | | | |

### Example 15: Incubation of Cord Blood Cells with p300/-Catenin Antagonist Expanded NSG Mice Engraftable Cells

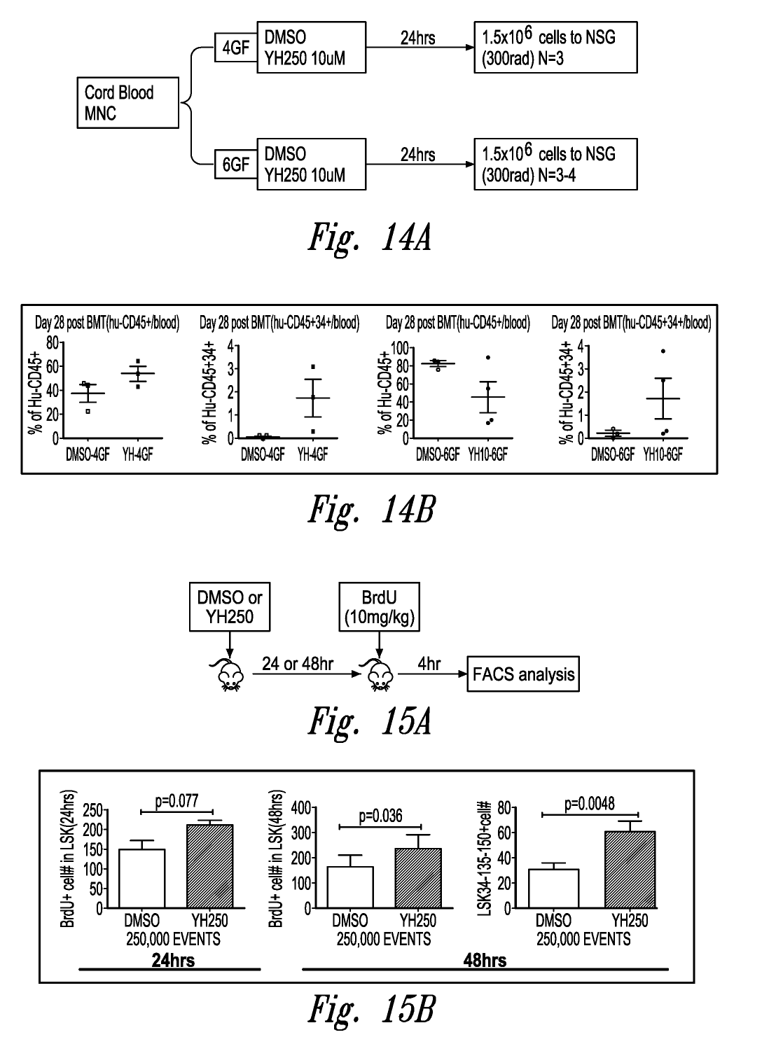

To examine p300/β-catenin antagonist YH250 effects on cord blood HSC in BMT, cord blood cells were incubated with 4GF or 6GF medium and 10 μM of YH250 for 24 hrs, then cells were recovered and transplanted to irradiated NOD.Cg-Prkdc^{scid}IL1rg^{tmlwjl}Tg(CMV-IL2, CSF2, KITLG)1Eva, MloySzJ; JAX stock#013062) mice, these are transgenic NSG mice expressing human interleukin-3, human GM-CSF and human stem cell factor. Blood were collected at day 28^{th} post BMT to determine engraftment. As depicted in FIG. 14, there are more human CD34posit cells from YH250 treated cells compared with DMSO treated cells.

### Example 16: p300/Catenin Antagonists Stimulated LTR-HSC Proliferation In Vivo

P300/-catenin antagonists stimulate HSPC proliferation both in vitro and in vivo. In this study, 24 or 48 hrs post either DMSO or YH250 administration, BrdU was given to animals and bone marrow cells were isolated and analyzed to determine HSPC proliferation (FIG. 15A). Bone marrow cells were stained with antibodies to lineage markers (CD3, CD4, CD8, B220, CD16, Gr-1, Mac-1), antibodies to Sca-1, c-kit, CD135, CD34, CD150 and BrdU. As shown in FIG. 15B, there are more BrdU positive cells in LTR-HSC (LSKCD34⁻CD135⁻CD150⁺ cells) of YH250 treated animals, indicating YH250 can stimulate HSPC proliferation in vivo.

### Example 17: Multiple Administration of p300/Catenin Antagonists Stimulated LTR-HSC In Vivo

The golden standard to determine HSPC number and activity is a competitive repopulation assay. The testing cells and competitor cells are mixed and transplanted to recipients to create an environment the testing cells compete with competitor cells to repopulate the recipients' hematopoietic system. The quantitative engraftment level reflects the testing cell stem/progenitor cell number.

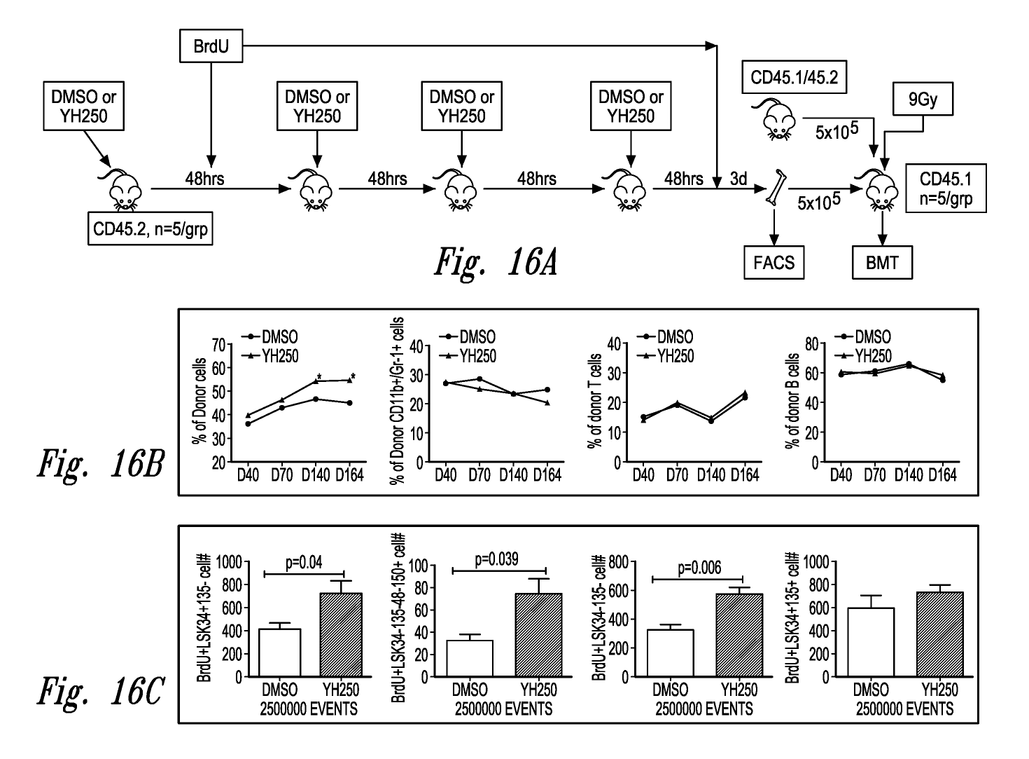

In this study, donor mice (CD45.2) were treated with YH250 or DMSO every 48 hs for 4 round, then, bone marrow cells from the treated mice were recovered and mixed with competitive cells from non-treated normal mice for BMT to lethally irradiated recipients (FIG. 16A). Blood were collected periodically from recipient mice to determine HSPC activities. As shown in FIG. 16B, both YH250 and DMSO treated mice bone marrow can repopulate full lineage blood cells for more than 4 month. At day 140 and 164 after BMT, the engraftment level from YH250 treated mouse bone marrow is significantly increased (day 140 and 164 vs day 40, p<0.01, n=5/group). This data suggest YH250 treated mouse bone marrow cells might contain more LTR-HSC. Also, the engraftment level of YH250 treated cells are higher than DMSO treated cells, although there is no statistical significance with limited animal numbers (n=5). At the end of experiment, bone marrow cells were FACS analyzed and the results show there are more BrdU positive cells in HSPC compartment (LSK34negt135neg, LSK34neg135neg48negt150posit, LSK34posit135negt cells) from YH250 treated animals (FIG. 16C).

### Example 18: p300/-Catenin Antagonists Stimulated HSPC Proliferation in Irradiated Mice

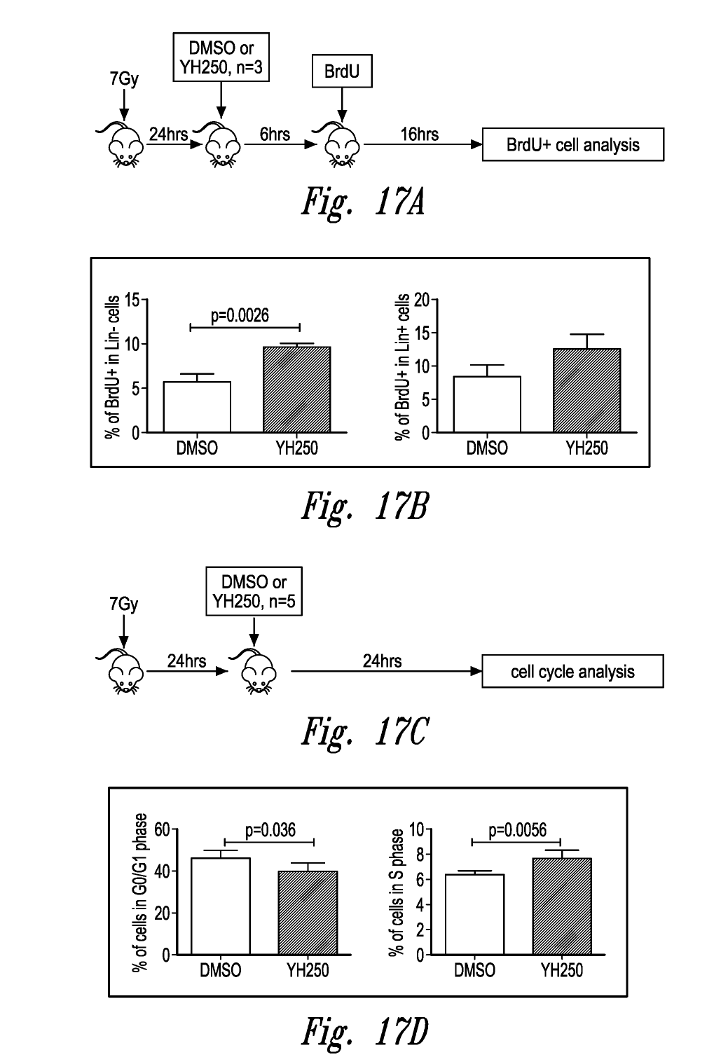

YH250 was also tested for stimulation of HSPC (hematopoietic stem/progenitor population) proliferation in irradiated animals. In this study, mice received 7Gy whole body radiation, DMSO or YH250 (s.c., 2 mg/Kg) were administrated 24 hours post radiation, followed with BrdU injection. FIG. 17A depicts the experimental procedure. FIG. 17B shows YH250 treated mice bone marrow have more BrdUpost cells in lineage negative (Lin) population, suggesting more lineage negative cells are stimulated by YH250 into cell proliferation. FIG. 17C, shows experimental procedure for cell cycle analysis in animals received 7Gy irradiation and then YH250 or DMSO. There are more bone marrow cells in S phase in animals received YH250 treatment (FIG. 17D, right panel). Then, YH250 was administrated to 7Gy irradiated animals and 4 days later, bone marrow cells were isolated for FACS analysis for HSC markers (FIG. 17E). As shown in FIG. 17F, there are increased HSPC in YH250 treated mice (right panel, increased L⁻CD48⁻CD150⁺ cells; “Long Term Repopulating” hematopoietic stem cells LTR-HSC).

### Example 19: p300/Catenin Antagonist YH250 Stimulated LTR-HSC

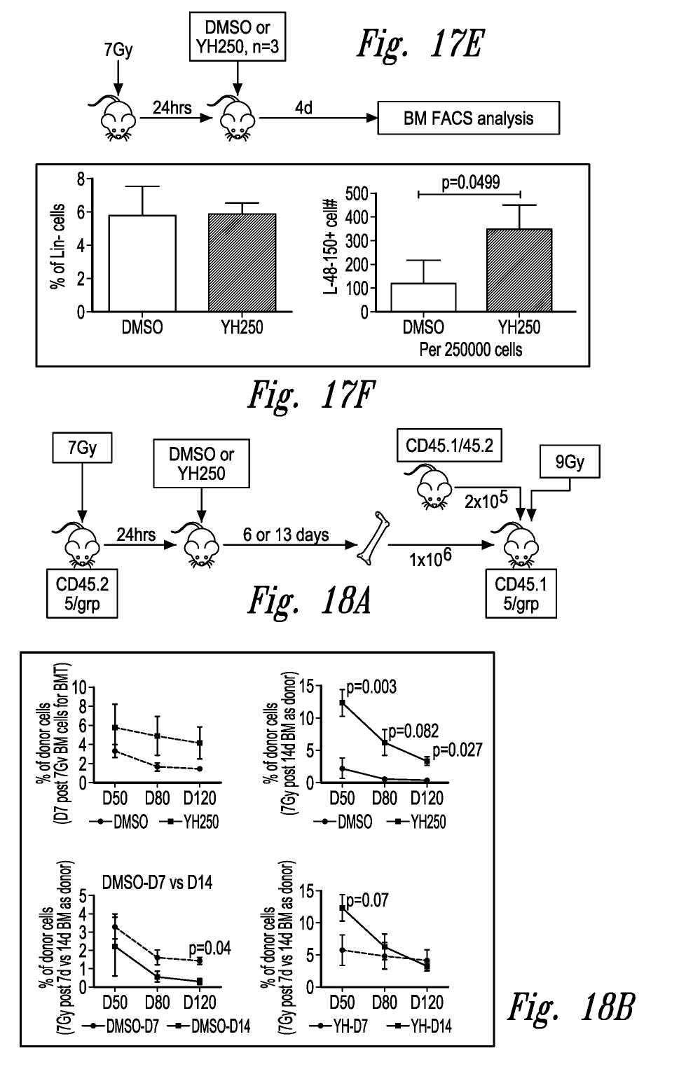

In the hematopoietic system, the LTR-HSC or pluripotent HSC sits atop the hematopoietic hierarchy. LTR-HSCs subsequently generate “Short Term Repopulating” hematopoietic stem cells (STR-HSC), and then progenitor cells. After radiation induced myeloid-ablation, there is a significant loss of progenitor cells. To investigate the target cell population in irradiated mice, YH250 or DMSO were administrated to 7Gy irradiated animals 24 hours post radiation. Then, 7 or 14 days post radiation (6 or 13 days after YH250 or DMSO administration), bone marrow cells were recovered from animals and competitive repopulation assay was performed to study YH250 function in stimulating HSPC proliferation. 1×10⁶ testing bone marrow cells were mixed with 2×10⁵ competitor cells (from non-irradiated mice) and injected into lethally irradiated mice (9Gy) (FIG. 18A). HSPC cell number will be reflected in the short-term (1-2 months after BMT) or long-term (≥4 month of BMT) engraftment. As shown in FIG. 18B, at 7^{th} day post radiation, bone marrow cells from YH250 treated animals give better engraftment compared with DMSO treated animals, both short term and long term. These results suggest YH250 expands HSPC by day 6 after one dose injection 24 hours post radiation. The same observation also applies to cells isolated 14th day post 7Gy radiation.

Notably, in YH250 treated mice, bone marrow cells recovered from either day 7 or day 14 post radiation give similar long-term engraftment (4.1±1.6 vs 3.4±0.57, p=0.70), but higher short term engraftment from cells recovered at day 14 post radiation (day 7 vs day 14, 5.8±2.4 vs 12.3±2.0, p=0.06). This result suggests that after one dose administration of YH250, the LRT-HSC expansion completed by day 6. The expanded LTR-HSC at day 6 post YH250 stimulation then starts to develop and give more STR-HSC by day 14 post radiation. There is no further LTR-HSC expanding from day 6 to day 13 post YH250. Thus, one dose of YH250 administrated 24 hours post 7Gy radiation stimulated symmetric non-differentiation LTR-HSC expansion and followed with STR-HSC expansion.

However, the situation is different with cells that received DMSO treatment. For both short term and long term repopulation, the cells recovered at day 7 post radiation show significantly better engraftment than cells recovered at day 14. In summary, YH250 treatment 24 hours post radiation stimulated HSC expansion.

### Example 20: p300/-Catenin Antagonists Accelerated Bone Marrow CFC Recovery

According to particular aspects of the present invention, if YH250 can stimulate 7Gy irradiated animal HSPC proliferation/expansion, the compound may accelerate the recovery of the hematopoiesis from radiation damage. We first tested if YH250 treatment accelerates bone marrow cell CFC activities, the more differentiated cells. After 7Gy radiation and either DMSO or YH250 administration 24 hours post radiation, bone marrow cells were recovered at different time points (day 6, 11, 13, 15 post treatment) and CFC assays were performed (FIG. 19A). As depicted in FIG. 19B, at 14^{th} day post radiation, cells from YH250 treated mice give significantly more colonies in CFC assay compared with DMSO treated mice. Consistent with increased colony numbers, there are more cells from plates of YH250 treated cells.

### Example 21: p300/-Catenin Antagonists Accelerated Peripheral Blood Cell Recovery from 7Gy Radiation

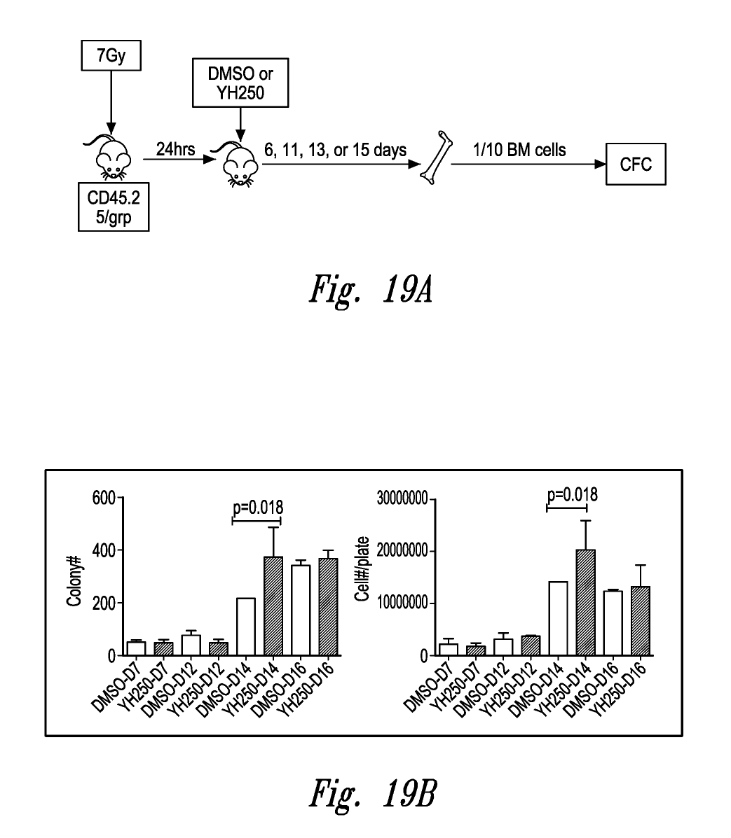

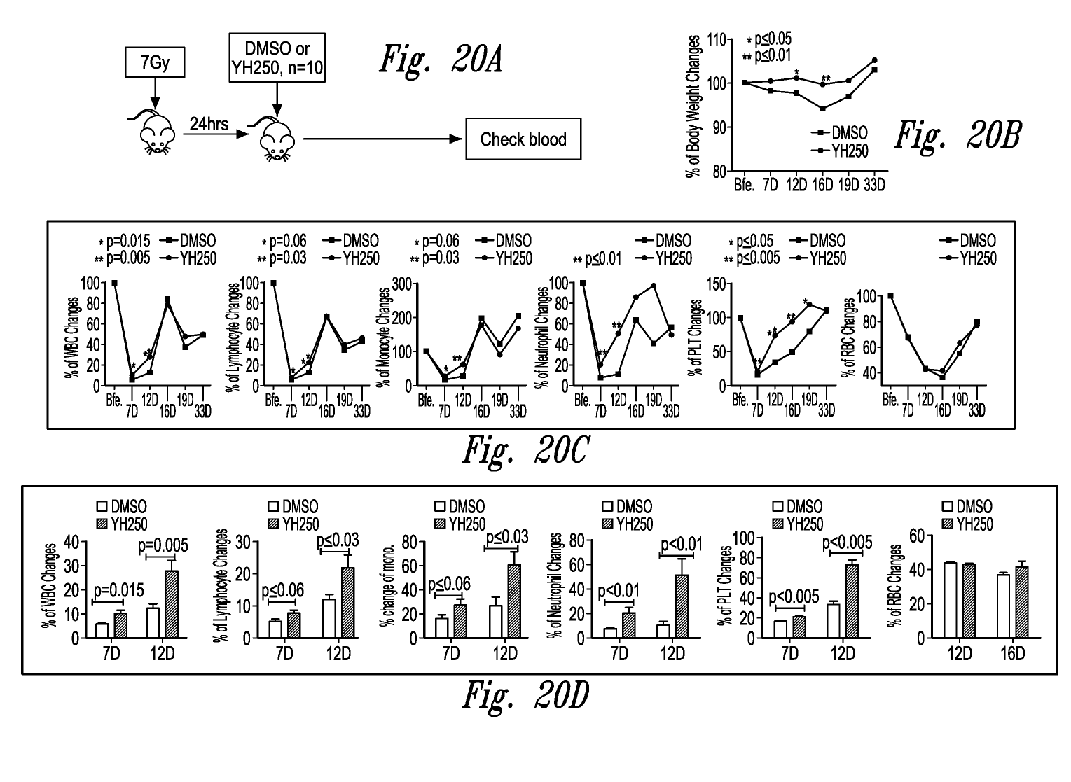

YH250 was then tested for stimulating blood count recovery in irradiated animals. In this experiment, animals received 7Gy whole body radiation and then, either YH250 or DMSO was administrated 24 hours post radiation. Animal body weight and peripheral blood counts were measured to monitor hematopoietic recovery (FIGS. 20A-20B). There is very little weight drop in YH250 treated animals while there is significantly weight loss in DMSO treated animals (FIG. 20B). Accelerated blood count recovery from YH250 treated animals was also observed. It is worth noting that it is a full lineage blood cell count recovery, including white blood cell (lymphocyte, monocyte and neutrophil), red blood cell and platelet (FIG. 20C). The neutrophil and platelet recovery is most significant. At nadir points, the blood cell count in YH250 treated animals are also significantly higher compared with DMSO treated animals (FIG. 20D), indicating accelerated recovery of blood cell count from YH250 treatment.

### Example 22: p300/Catenin Antagonist YH250 Rescued Animals from Lethal Dose Radiation

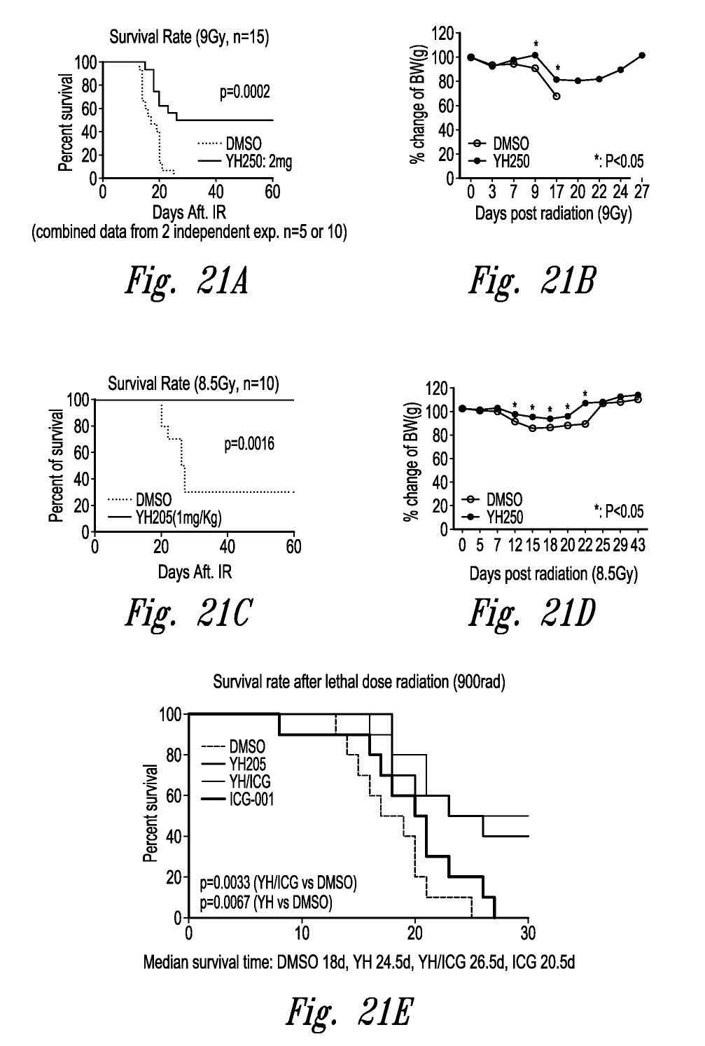

We next tested if YH250 can rescue animals from lethal dose radiation. Animals received either 9Gy (LD100/60, 100% animals died in 60 days post radiation) or 8.5Gy (LD70/60, 70% animals died in 60 days post radiation) whole body radiation, 24 hours later, DMSO or YH250 (2 mg/Kg) were administrated. As shown in FIG. 21A, all animals in DMSO treatment group died within 30 days post 9Gy radiation. However, YH250 (2 mg/Kg) administrated 24 hours post radiation rescued 50% of animals (Kaplan-Meier analysis p=0.0002). YH250 treated animals also show less body weight drop (FIG. 21B).

With 8.5Gy radiation, all the animals (100%) survived in YH250 treatment group while there are only 30% animals survived in DMSO treatment group (p=0.0016, FIG. 21C). Again, YH250 treatment resulted in less body weight drop compared with DMSO treatment (FIG. 21D, *: p<0.05). In principle, to optimize radiation remediation therapy, it should be advantageous to first (a) symmetrically expand the remaining viable stem cell pool (either HSC or ISC), using a method to enhance symmetric non-differentiative proliferation, i.e., by blocking the p300/catenin interaction thereby enhancing CBP/catenin signaling and (b) subsequently induce the differentiation of the stem cell pool via asymmetric divisions to enhance tissue repair and regeneration as rapidly as possible using a CBP/catenin antagonist. We therefore decided to test sequential administration of the p300/catenin antagonist YH250, followed by subsequent administration of the CBP/catenin antagonist ICG-001. An additional potential therapeutic benefit utilizing a CBP/catenin antagonist would be protection against fibrosis, which is a common chronic complication associated with radiation damage. CBP/catenin antagonists have previously demonstrated efficacy in multiple pre-clinical models of fibrosis in lung, kidney, liver etc. We thus treated mice with either vehicle, YH250 (single 2 mg/Kg s.c., injection at 24 h post irradiation), ICG-001 (single 50 mg/Kg s.c., injection at 24 h post irradiation) or the combination of YH250/ICG-001, with YH250 (2 mg/Kg) given 24 h post irradiation followed by ICG-001 (50 mg/Kg) injection for 5 consecutive days starting at 48 h post-irradiation. After irradiation and the corresponding treatments, no special care was provided. The life-span after irradiation was observed on a daily basis and recorded. The survival curves are shown in FIG. 21E. Mice that received either YH250 or a combination of YH250 and ICG-001 showed significantly extended life-spans (p=0.0067 and 0.0033 for the YH 250 and YH250/ICG-001 groups versus the control group respectively).

### Example 23: p300/Catenin Antagonists Did not Exhaust LTR-HSC

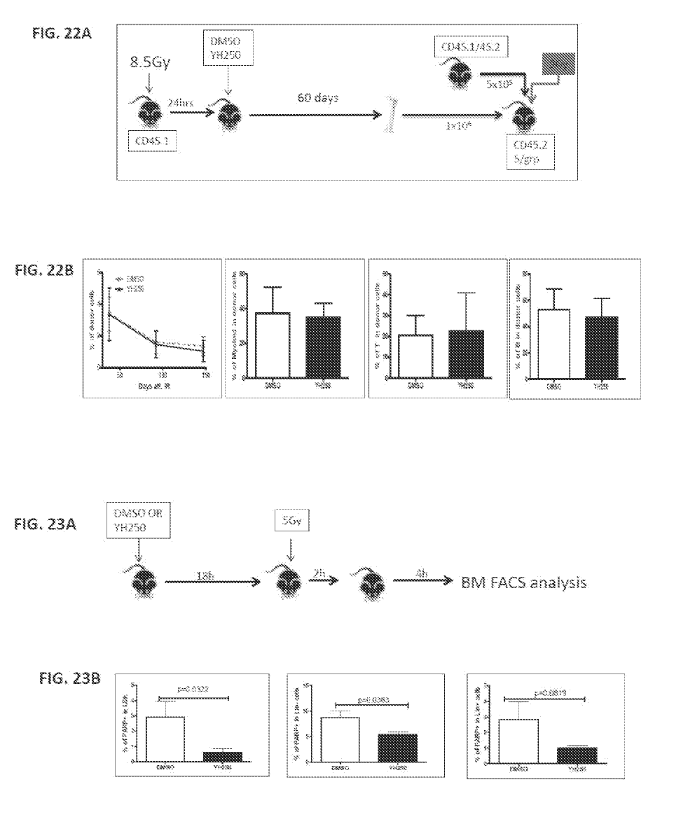

To test if YH250 treatment caused HSPC exhaustion, bone marrow cells were isolated from animals in DMSO or YH250 treatment group in 8.5Gy irradiated experiment (FIG. 21C) and competitive repopulation assay (FIG. 22A) were performed. As shown in FIG. 22B, the engraftment level is similar between DMSO or YH250 treated animals. There is no lineage bias in peripheral blood. Taking together, these results indicate that one dose administration of YH250 at 24 hours post lethal dose radiation stimulates HSPC proliferation, accelerates full lineage blood cell recovery and does not exhaust HSPC.

### Example 24: p300/Catenin Antagonists Attenuated Radiation Induced Cell Apoptosis in HSPC

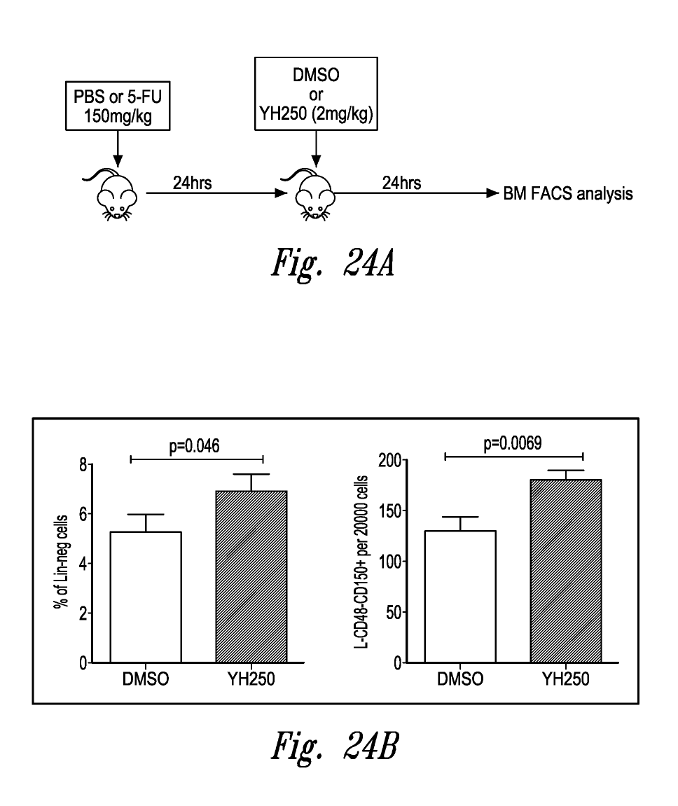

Since p300/-catenin antagonists such as YH250 up-regulate survivin gene expression, YH250 was tested for the ability to attenuate radiation induced cell apoptosis by detecting cleaved PARP (Poly ADP ribose polymerase-1) using anti-PARP antibody and FACS analysis (BD Bioscience, Cat#562253, anti-PARP clone F21-852). DMSO or YH250 were administrated 18 hours before 5Gy radiation. 6 hours after radiation, bone marrow cells were isolated and stained for cell surface markers (lineage markers, Sca-1, c-kit) and anti-PARP (FIG. 23A). As depicted in FIG. 23B, there are significantly less apoptosis in LSK, lineage-negt and lineage-posit population in YH250-treated animals.

### Example 25: p300/-Catenin Antagonists Stimulated HSPC Proliferation in 5-FU Treated Animals

p300/-catenin antagonists, such as YH250, were also tested for the ability to stimulate bone marrow stem/progenitor cell proliferation in chemotherapeutic drug-treated (e.g., 5-FU) animals. As depicted in FIG. 24A, animals were given PBS or 5-FU (150 mg/kg). 24 hours later, either DMSO or YH250 (2 mg/kg) were administrated to animals. Then, bone marrow cells were isolated one day later for FACS analysis. As depicted in FIG. 24B, both lineage negative and Lin-CD48-CD150+ cell numbers are significantly higher in YH250 treated mice than in DMSO treated mice.

### Example 26: p300/-Catenin Antagonists Facilitated Animal Blood Cell Recovery from Repeated 5-FU Administration

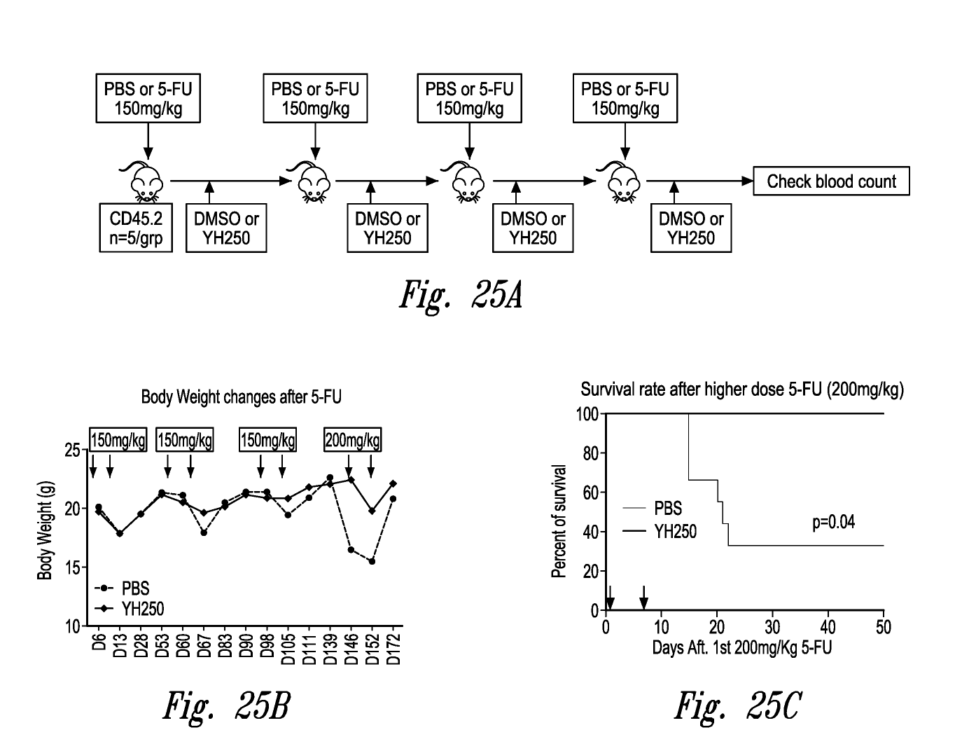

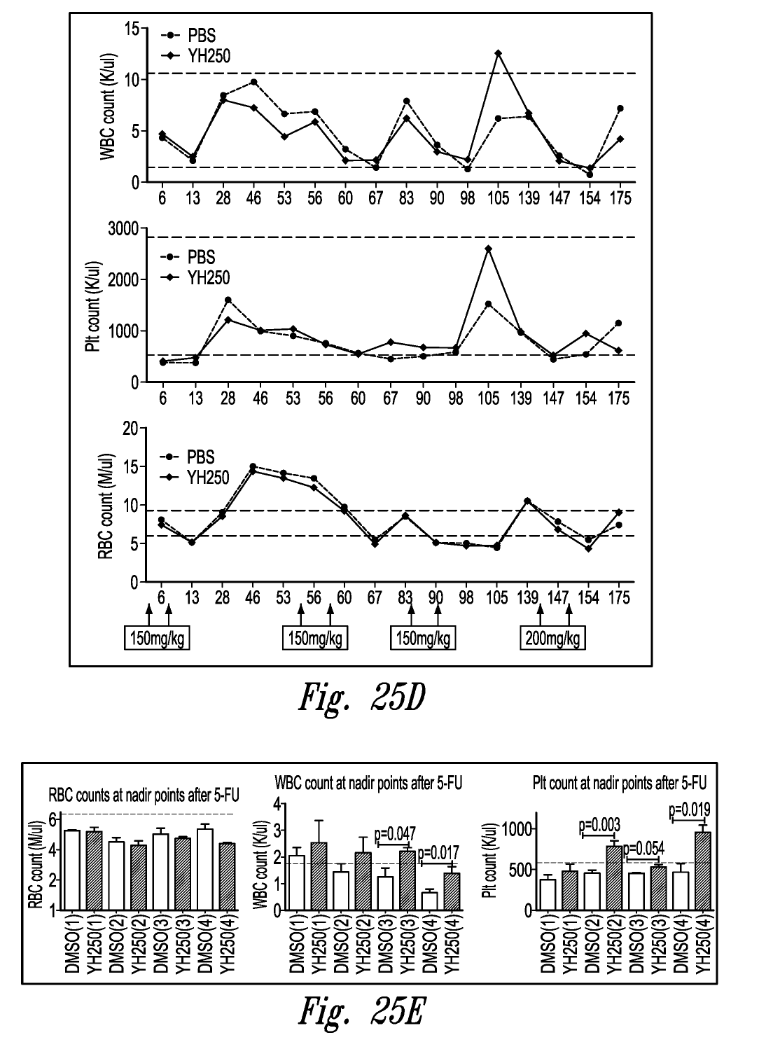

To test if p300/-catenin antagonists can also promote multi-dosing chemotherapeutic drug induced bone marrow inhibition, 4 rounds of 5-FU (two doses in each round) administration were given to CD45.2 female mice. 150 mg/kg of 5-FU was given to mice for the first three rounds and then 200 mg/kg for the last round (4^{th} round). The second dose of 5-FU was 6 days apart from the first dose of 5-FU in each round. Either DMSO or YH250 (2 mg/kg) were given to animals 24 hours after the first dose of 5-FU in each round of 5-FU administration (FIG. 25A). Animal body weight, survival rate and blood count changes were monitored. As depicted in FIG. 25B, YH250 treated animals show less weight drop from second round of 5-FU administration, especially in the last round of higher dose 5-FU administration, YH250 treated animals show significantly more body weight drop. At higher dose of 5-FU (200 mg/kg), 60% of animals died in DMSO group while all animals survived in YH250 group (FIG. 25C, p=0.04 by Kaplan-Meier analysis). FIG. 25D shows the blood count changes during 4 rounds of 5-FU administration. After each round of 5-FU administration, animals were allowed to recover to normal blood count before next round of 5-FU administration. FIG. 25E depicts the blood count at nadir points after each round of 5-FU administration. There is no significant effect on RBC from YH250 administration. However, WBC and platelet are significantly higher at nadir point in YH250 treated animals especially at the last three rounds of 5-FU administration.

### Example 27: Repeated Administration of p300/-Catenin Antagonists in Multi-Dosing 5-FU Treated Animals Did not Affect HSPC Function

At the last round of 5-FU treatment from FIG. 25A, bone marrow cells from survived animals were isolated (CD45.2) and mixed with competitor cells (CD45.1/CD45.2 hybrid) and injected to CD45.1 mice for competitive repopulation assay. This is to assess whether YH250, by multiple uses in this experiment setting, will result in changes of HSC activity and lead to HSC exhaustion (FIG. 26A). As depicted in FIG. 26B, bone marrow cells from multi-dose YH250 administrated mice show similar engraftment level as bone marrow cells from multi-dose DMSO administrated mice. Additionally, there is no lineage bias in both short-term and long-term repopulation.

### Example 28: YH249 Showed Similar Function as YH250 with Respect to HSC

YH250 and YH249 are structurally similar as shown in Table 1. In fact, they also show similar biological functions with respect to HSPC. When mice bone marrow cells were incubated with DMSO, or YH249 or YH250 for 4 hours and then subject to CFC assay, YH249 treated bone marrow cells give similar colony number in plate as DMSO but larger colonies that resulted in higher cell number in each plate (Table 6).

TABLE 6

| | DMSO | YH249 | YH250 |
|---|---|---|---|
| CFC#/plate | 26 | 27 | 29 |
| CELL#/plate | 3.4 × 10⁵ | 5.2 × 10⁵ | 9.5 × 10⁵ |

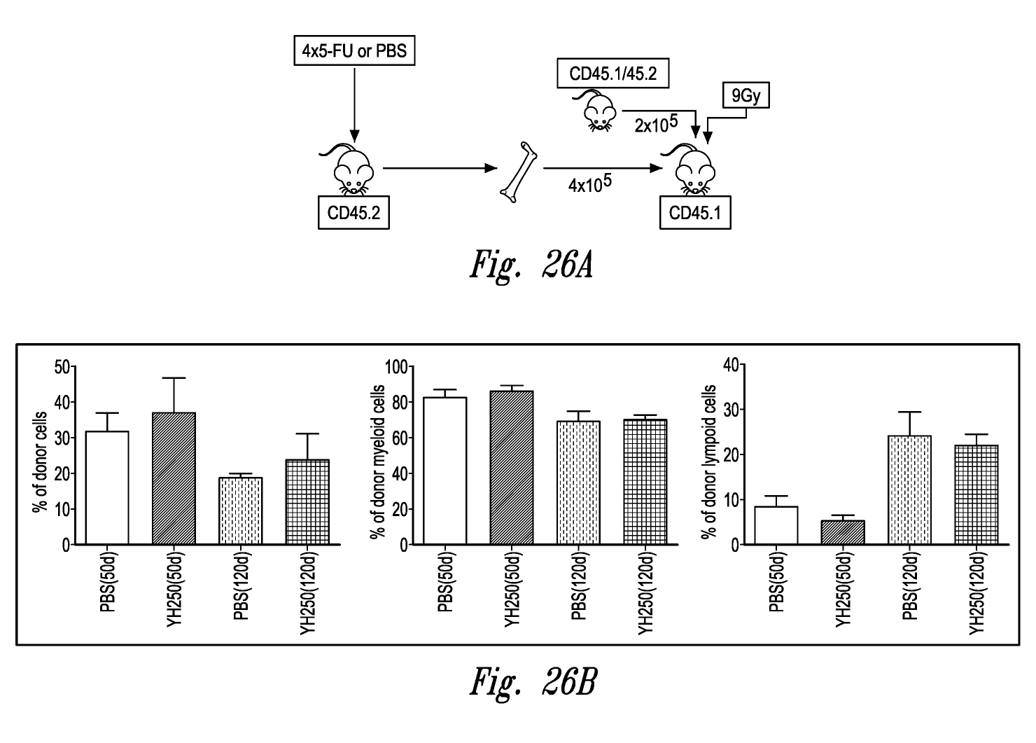

In a further study, female CD45.1 mice bone marrow cells were incubated with DMSO, or YH249 (0.5 μM) or YH250 (0.5 μM) for 4 hrs in QBSF-58 media, then 2×10⁴ cells were plated in QBSF-58 medium containing SCF, TPO, Flt3 and IL-6 (10 ng/ml for each) for 14 days. Cells were then recovered from plate for FACS analysis. As shown in FIG. 27, the lineage negative cell in this condition are 9%, 17% and 33% for DMSO, YH249 and YH250 treated cells, respectively. Correspondingly, there are higher LSK cells (52%, 60% and 70% respectively for DMSO, YH249 and YH250). From every 10,000 analyzed cells, LSKCD34negt cell numbers are 437, 663 and 2051 for DMSO, YH249 and YH250 treated cells, respectively.

### Example 29: p300/p-Catenin Antagonists Maintained Pluripotency of Mouse and Human ES Cells and Human iPS Cells

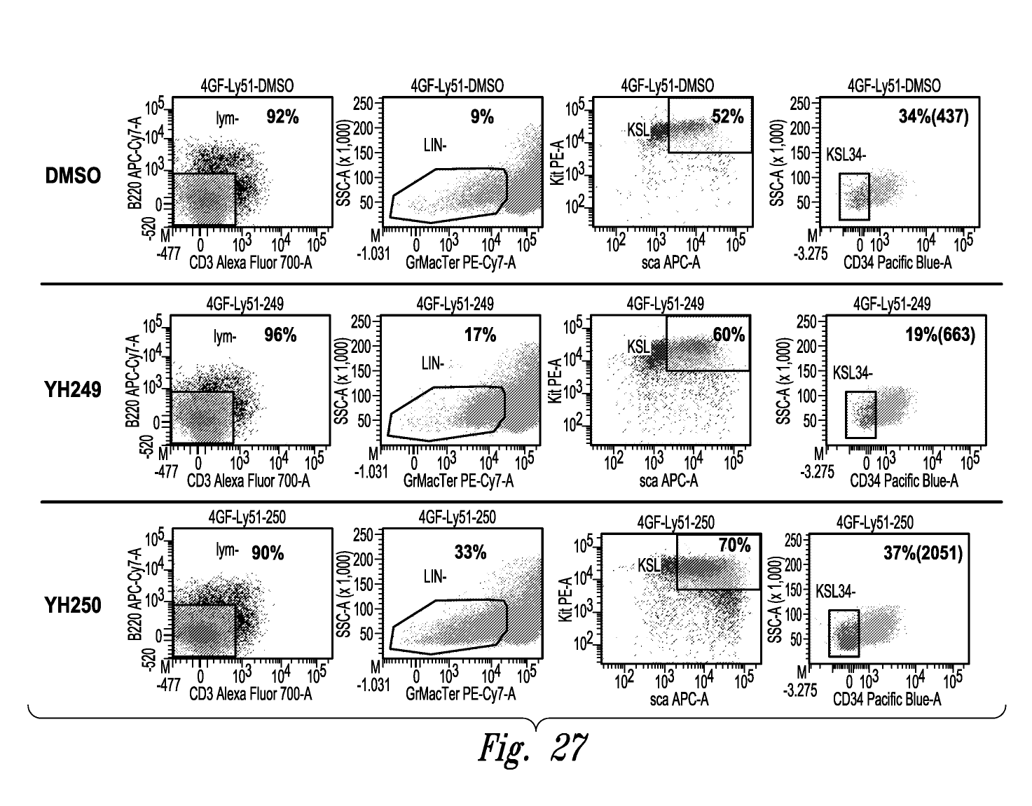

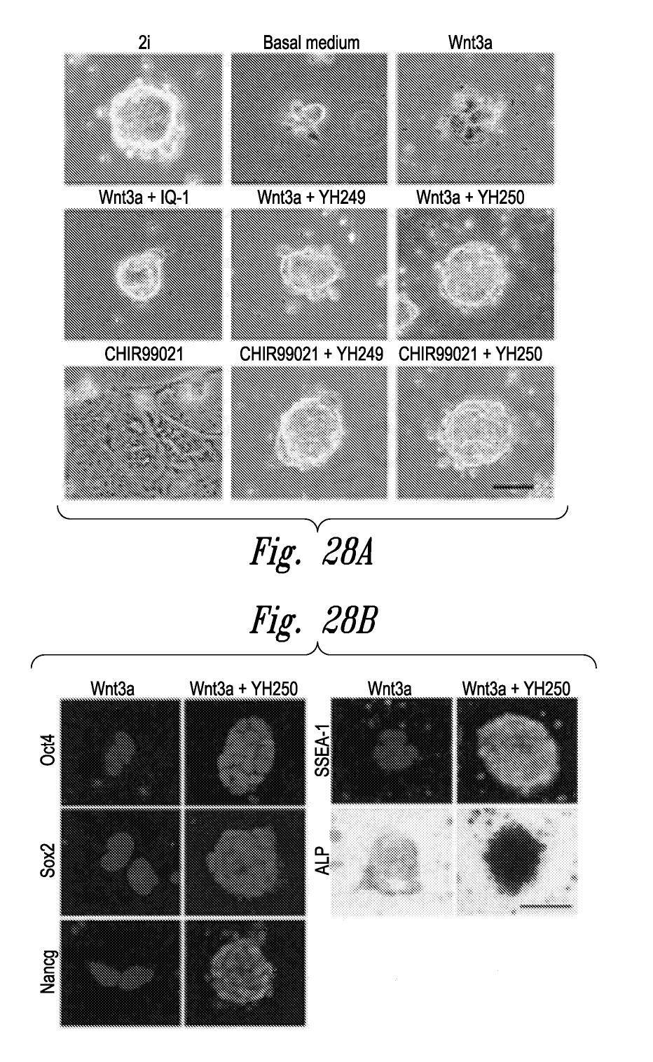

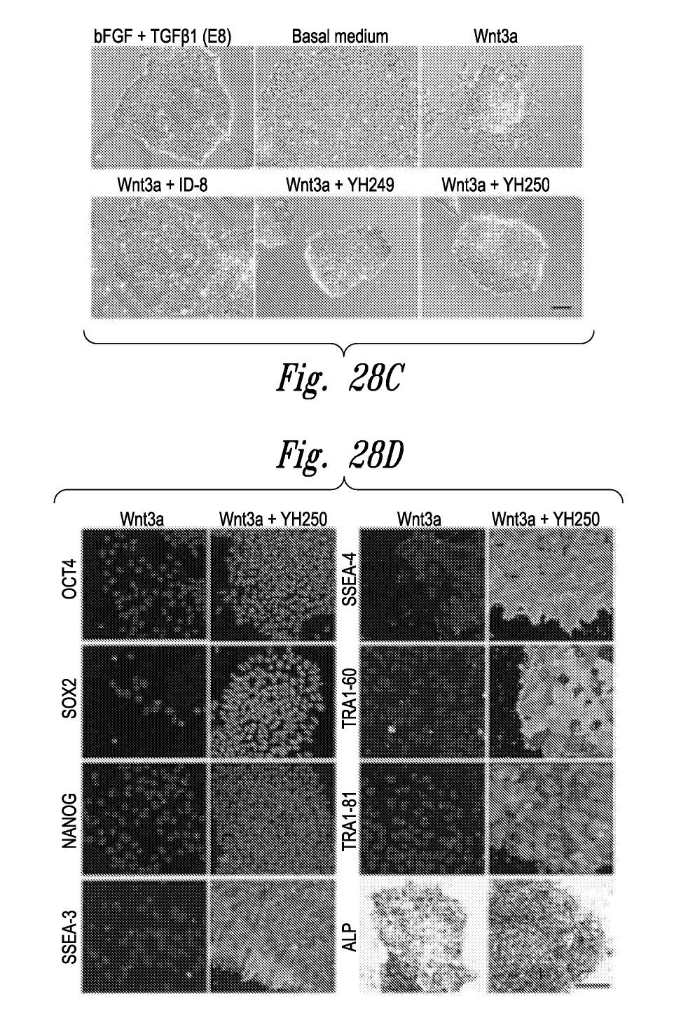

We have previously reported that the small molecule IQ-1 could maintain long term mESC pluripotency in a Wnt dependent manner {Miyabayashi, T., et al., Proc. Natl. Acad. Sci., 104, 5668-5673, 2007). We also determined that IQ-1 indirectly, via binding to the PR72/130 subunit of PP2A, inhibited the p300/β-catenin interaction (Id). Therefore, we anticipated that YH249/250 should also maintain mESC pluripotency in a Wnt dependent manner. To test his, R1 mESCs were cultured under feeder free conditions in the presence or absence of YH249/250 for 5 days. The mESCs grown in the presence of YH249/250 and Wnt3a maintained undifferentiated colony morphology (FIG. 28a, middle panel), whereas Wnt3a alone was not sufficient to maintain pluripotency for this period of time (FIG. 28a, b). Pluripotency markers, Oct4, Nanog, Sox2, SSEA-1 and ALP, were all expressed in the cells treated with Wnt3a and YH250 (FIG. 28b). Moreover, Wnt3a could be replaced with the selective GSK-3 inhibitor CHIR99021, which activates the Wnt signaling pathway (FIG. 28a, lower panel). Next, we examined whether YH249/250 could also maintain hPSC pluripotency as does the small molecule indirect p300/β-catenin antagonist ID-8 (Hasegawa K., et al., Stem Cells Transl. Med., 1, 467-472, 2005). In the event, similarly to ID-8, the combination of Wnt3a and YH249/250 provided morphologically undifferentiated colonies of H9 human embryonic stem cells (hESCs) without FGF and TGF (FIG. 28c). hPSC markers were detected by ALP staining and immunofluorescence (FIG. 28d). In addition to H9 cells, two other hESC lines and a human induced pluripotent stem (iPS) cell line could be also maintained undifferentiated morphology utilizing the same conditions (not shown). These results further confirm that either direct or indirect disruption of the p300/β-catenin interaction is sufficient to maintain the pluripotency of both mouse and human pluripotent stem cells, despite their “ground state” differences, under conditions where Wnt signaling is activated.

Specifically, FIG. 28A-28D, show maintenance of ESC's pluripotency by YH249 and YH250. FIGS. 28A and 28B show that mESCs maintain pluripotency when cultured with YH249 and YH250. 28A, Typical colony morphologies of RI mouse ES cells cultured 5 days in [upper panel] 3 μM CHIR99021 and 1 μM PD0325901 (2i) medium, basal medium (2i medium without CHIR99021 and PD0325901), basal medium supplemented with 50 ng/ml Wnt3a alone, [middle panel] combination of 50 ng/ml Wnt3a and 2 μM IQ-1, 500 nM YH249 or 200 nM YH250, [lower panel] 3 μM of CHIR99021 alone, and combination of 3 μM CHIR99021 and 500 nM YH249 or 200 nM YH250. FIG. 28B shows alkaline phosphatase (ALP) staining and immunostaining of pluripotency cell markers in RI cells maintained in basal medium supplemented with 50 ng/ml Wnt3a alone and combination of Wnt3a and 200 nM YH250. The blue signal (Wnt3a panels) represents DAPI nuclear staining. Bars indicate 50 m. FIGS. 28C and 28D show that hESCs maintain pluripotency when cultured with YH249 and YH250. FIG. 28C shows typical colony morphologies of H9 human ES cells cultured 5 days in [upper panel] basal medium supplemented with 100 ng/ml bFGF and 2 ng/ml TGFβl (E8 culture medium), basal medium, 25 ng/ml Wnt3a alone, and [lower panel] combination of 25 ng/ml Wnt3a and 500 nM ID-8, 500 nM YH249 or 200 nM YH250. FIG. 28D shows ALP staining and immunostaining of pluripotency cell markers in H9 cells maintained with basal medium supplemented with 25 ng/ml Wnt3a alone and combination of 25 ng/ml Wnt3a and 200 nM YH250. The blue signal (Wnt3a panels) represents DAP1 nuclear staining. Scale bars indicate 100 am.

### EQUIVALENTS

Although the foregoing has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

The disclosure illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A compound of Formula I 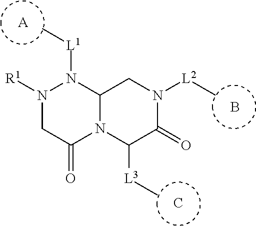
or a pharmaceutically acceptable salt thereof,
wherein
R¹ is hydrogen or C₁-C₆ alkyl;
ring A is cyclohexyl or phenyl, optionally substituted;
ring B is aryl or a nitrogen-containing heteroaryl, optionally substituted;
ring C is hydroxyphenyl;
each L¹, L² and L³ is independently C₁-C₆ alkyl, —CONR²— or —CONR²—X—C₁-C₆ alkyl-, each optionally substituted; R² is hydrogen or C₁-C₆ alkyl; and
X is a bond or a 5-6 membered heterocycle containing up to 3 ring heteroatoms.

2. The compound of claim 1, wherein ring A is cyclohexyl or phenyl substituted with 1-4 groups selected from halo; C₁-C₆ alkyl, optionally substituted with 1-6 halo; C₁-C₆ alkoxy, optionally substituted with 1-6 halo; wherein two adjacent alkyl or alkoxy groups can join to form a 5- or 6-membered carbocyclic or heterocyclic ring.

3. The compound of claim 1, wherein ring A is

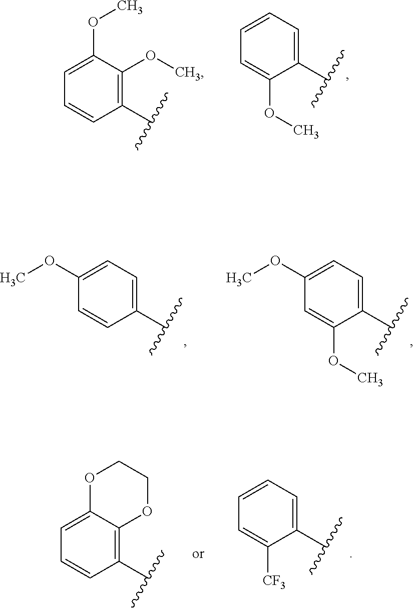

4. The compound of claim 1, wherein ring B is phenyl, naphthyl, pyridyl, imidazolyl or quinolyl: wherein each is optionally substituted with 1-4 groups selected from halo: C₁-C₆ alkyl, optionally substituted with 1-6 halo; C₁-C₆ alkoxy, optionally substituted with 1-6 halo: NR³R⁴, wherein R³ and R⁴ are independently hydrogen or C₁-C₆ alkyl; or heterocyclyl, optionally substituted with C₁-C₆ alkyl; and two adjacent alkyl, alkoxy or NR³R⁴ groups can join to form a 5- or 6-membered carbocyclic or heterocyclic ring.

5. The compound of claim 4, wherein ring B is 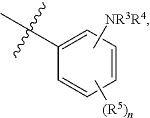
R⁵ is halo and n is 1, 2 or 3.

6. The compound of claim 4, wherein ring B is 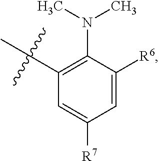
and R⁶ and R⁷ are independently halo.

7. The compound of claim 1, wherein ring C is

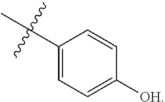 [SMILES: *c1ccc(O)cc1]

8. The compound of claim 1, wherein L¹ is

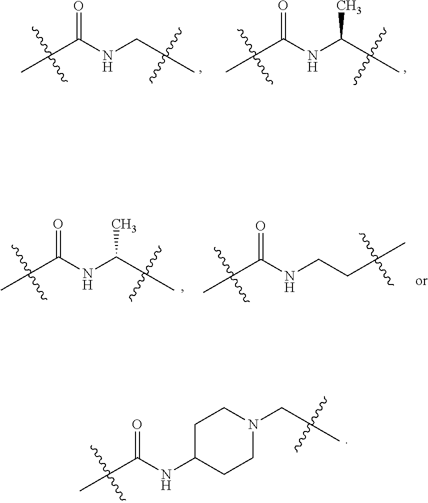

9. The compound of claim 8, wherein L¹ is

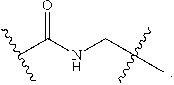 [SMILES: *CNC(*)=O]

10. The compound of claim 1, wherein L² and L³ are —CH₂—.

11. The compound of claim 1, wherein the compound is a compound in Table 1 or a pharmaceutically acceptable salt thereof.

12. The compound of claim 1, wherein the compound is 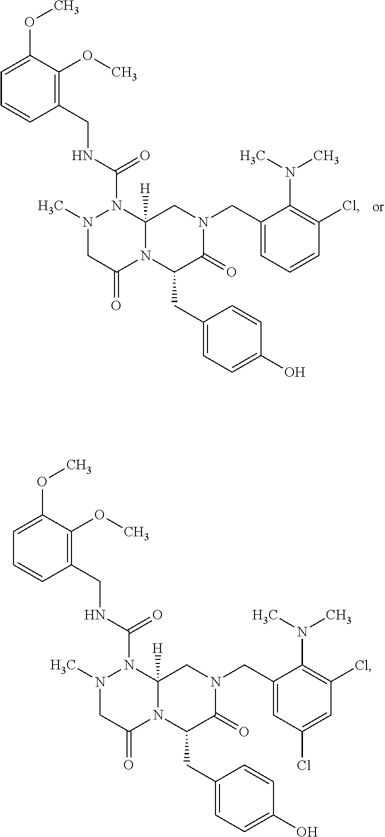 [SMILES: COc1cccc(CNC(=O)N2[C@H]3CN(Cc4cccc(Cl)c4N(C)C)C(=O)[C@H](Cc4ccc(O)cc4)N3C(=O)CN2C)c1OC ; COc1cccc(CNC(=O)N2[C@H]3CN(Cc4cc(Cl)cc(Cl)c4N(C)C)C(=O)[C@H](Cc4ccc(O)cc4)N3C(=O)CN2C)c1OC]
or a pharmaceutically acceptable salt thereof.

13. A pharmaceutical composition comprising a compound, or a pharmaceutically acceptable salt thereof, of claim 1 and a carrier, such as a pharmaceutically acceptable carrier and/or excipient.

14. A method for increasing hematopoiesis in a subject in need thereof, comprising administering to the subject an effective amount of a compound of Formula I 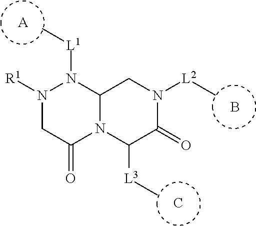
or a pharmaceutically acceptable salt thereof,
wherein
R¹ is hydrogen or C₁-C₆ alkyl;
ring A is cyclohexyl or phenyl, optionally substituted;
ring B is aryl or a nitrogen-containing heteroaryl, optionally substituted;
ring C is phenyl substituted with hydroxyl;
each L¹, L² and L³ is independently C₁-C₆ alkyl, —CONR²— or —CONR²—X—C₁-C₆ alkyl-, each optionally substituted; R² is hydrogen or C₁-C₆ alkyl; and
X is a bond or a 5-6 membered heterocycle containing up to 3 ring heteroatoms.

15. The method of claim 14, wherein the subject is an animal, a mammal or a human.

16. The method of claim 14, wherein the subject is suffering from, or is susceptible to, decreased or depressed hematopoiesis or blood cell levels.

17. The method of claim 16, wherein the decreased or depressed hematopoiesis or blood cell levels are caused by chemotherapy, radiation therapy, bone marrow transplantation therapy, accidental exposure to radiation or congenital anemia.

18. The method of claim 14, further comprising administering to the subject an effective amount of a CBP/β-, and/or γ-catenin antagonist that promotes hematopoietic stem cell (HSC) differentiation, wherein the CBP/β-, and/or γ-catenin antagonist is not a compound of Formula I or a pharmaceutically acceptable salt thereof.

19. The method of claim 18, further comprising protection against fibrosis, which is a common chronic complication associated with radiation damage.

20. The method of claim 18, wherein the CBP/β-, and/or γ-catenin antagonist is PRI-724 or ICG-001.

21. The method of claim 14, further comprising administering to the subject an effective amount of growth or differentiation factor.

22. A method for enhancing expansion of a hematopoictic stem cell (HSC), comprising contacting the HSC with a compound, wherein the compound is of Formula I 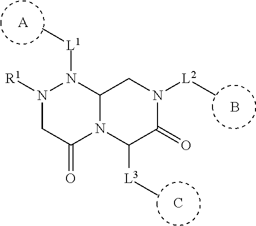
or a pharmaceutically acceptable salt thereof,
wherein
R¹ is hydrogen or C₁-C₆ alkyl;
ring A is cyclohexyl or phenyl, optionally substituted;
ring B is aryl or a nitrogen-containing heteroaryl, optionally substituted;
ring C is phenyl substituted with hydroxyl;
each L¹, L² and L³ is independently C₁-C₆ alkyl, —CONR²— or —CONR²—X—C₁-C₆ alkyl-, each optionally substituted; R² is hydrogen or C₁-C₆ alkyl; and
X is a bond or a 5-6 membered heterocycle containing up to 3 ring heteroatoms.

23. The method of claim 22, wherein the cell is autologous or allogeneic and the contacting is in vitro or in vivo.

24. The method of claim 22, further comprising contacting the HSC with a CBP/β-, and/or γ-catenin antagonist that promotes hematopoietic stem cell (HSC) differentiation, wherein the CBP/β-catenin antagonist is not a compound of Formula I or a pharmaceutically acceptable salt thereof.

25. The method of claim 24, wherein the CBP/β-, and/or γ-catenin antagonist is PRI-724 or ICG-001.

26. A method for inhibiting an interaction between a β-, and/or γ-catenin protein and a p-300 protein in a subject, the method comprising administering to the subject an effective amount of a compound of Formula I 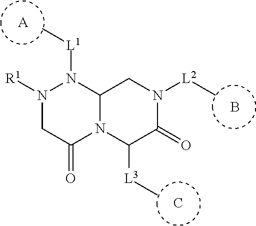
or a pharmaceutically acceptable salt thereof,
wherein
R¹ is hydrogen or C₁-C₆ alkyl;
ring A is cyclohexyl or phenyl, optionally substituted;
ring B is aryl or a nitrogen-containing heteroaryl, optionally substituted;
ring C is phenyl substituted with hydroxyl;
each L¹, L² and L³ is independently C₁-C₆ alkyl, —CONR²— or —CONR²—X—C₁-C₆ alkyl-, each optionally substituted: R² is hydrogen or C₁-C₆ alkyl; and
X is a bond or a 5-6 membered heterocycle containing up to 3 ring heteroatoms.

27. The method of claim 26, further comprising administering to the subject a CBP/β-, and/or γ-catenin antagonist that promotes hematopoietic stem cell (HSC) differentiation, wherein the CBP/β-, and/or γ-catenin antagonist inhibits the interaction between the β-catenin or γ-catenin and a CBP protein in the subject and is not a compound of Formula I or a pharmaceutically acceptable salt thereof.

28. The method of claim 26, wherein the subject is an animal, a mammal, or a human.

29. A method for maintaining pluripotency of embryonic stem (ES) cells and induced pluripotent stem (iPS) cells, comprising culturing ES cells or iPS cells in the presence of an effective amount of a compound of Formula I 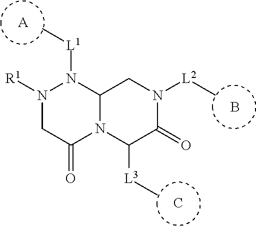
or a pharmaceutically acceptable salt thereof,
wherein
R¹ is hydrogen or C₁-C₆ alkyl;
ring A is cyclohexyl or phenyl, optionally substituted;
ring B is aryl or a nitrogen-containing heteroaryl, optionally substituted;
ring C is phenyl substituted with hydroxyl;
each L¹, L² and L³ is independently C₁-C₆ alkyl, —CONR²— or —CONR²—X—C₁-C₆ alkyl-, each optionally substituted; R² is hydrogen or C₁-C₆ alkyl; and
X is a bond or a 5-6 membered heterocycle containing up to 3 ring heteroatoms.

30. The method of claim 29, wherein the ES cells are Human or murine, and the iPS are Human.

31-32. (canceled)
